(19) United States
(12) Patent Application Publication
(10) Pub. No.: US 2022/0249538 A1
(43) Pub. Date: Aug. 11, 2022

(54) TREATMENT AGENT FOR SEX HORMONE-INSENSITIVE GREB1-POSITIVE TUMORS

(71) Applicant: OSAKA UNIVERSITY, Suita-shi, Osaka (JP)

(72) Inventors: Akira Kikuchi, Suita-shi, Osaka (JP); Shinji Matsumoto, Suita-shi, Osaka (JP); Taku Yamamichi, Suita-shi, Osaka (JP)

(21) Appl. No.: 17/595,703

(22) PCT Filed: May 22, 2020
(86) PCT No.: PCT/JP2020/020274

§ 371 Date: Nov. 22, 2021

(30) Foreign Application Priority Data
May 23, 2019 (JP) 2019-097051

(51) Int. Cl. A61K 31/7105; G01N 33/574; C12Q 1/6886; A61P 35/00; A61K 31/713

(52) U.S. Cl. CPC A61K 31/7105; G01N 33/57484; C12Q 1/6886; A61P 35/00; A61K 48/00; C12Q 2600/106; C12Q 2600/156; C12Q 2600/158; A61K 31/713

(57) ABSTRACT

The purpose of the present invention is to provide a novel tumor treatment agent. In GREB1-positive tumors that do not exhibit sex hormone sensitivity, GREB1 is a downstream target gene of the Wnt/β-catenin signal. Through the use as an active ingredient of a substance that can inhibit GREB1 expression, the growth of GREB1-positive tumor cells that do not exhibit sex hormone sensitivity can be effectively inhibited and such a tumor can be treated.

### TECHNICAL FIELD

The present invention relates to a therapeutic agent for a sex hormone insensitive GREB1 (Growth regulation by estrogen in breast cancer)-positive tumor (GREB1-positive tumor that does not show sensitivity to sex hormones). Further, the present invention relates to a testing method for hepatoblastoma, hepatocellular cancer, malignant melanoma, and neuroblastoma using the expression level of GREB1 as an indicator.

### BACKGROUND ART

Hepatoblastoma is a malignant tumor that occurs in the liver of a child. Hepatoblastoma, most of which occur before the age of 3 years, account for 90% of all malignant hepatic tumors of children younger than 4 years. In Japan, 30 to 40 new cases of hepatoblastoma occur each year, with an incidence of about 1 in 1,000,000 people in the world, and hepatoblastoma is considered as a rare cancer. Although causes of the development of hepatoblastoma are not known, it is found that approximately 70% of the patients have a deletion or an activation mutation in a region including exon 3 associated with β-catenin degradation, and hepatoblastoma is common to groups with familial adenomatous polyposis (FAP) with β-catenin accumulation. Thus, it is thought that activation of Wnt signal contributes to the pathological conditions.

In hepatoblastoma patients, since whether or not complete resection has been achieved has influence on the prognosis, the selection of therapeutic strategies depends on staging after surgery. Although the prognosis after complete resection is good in cases of disease stage (stage) I, the 5-year disease-free survival rate decreases to only 36% in cases of disease stage IV with distant metastasis. Conventionally, in the treatment of hepatoblastoma, chemotherapy and surgical resection are given in combination. In chemotherapies, although a regimen including cisplatin has been established as a standard therapy, severe side effects such as myelosuppression and renal insufficiency are problematic.

On the other hand, GREB1 was discovered as one of estrogen-induced genes in breast cancer cells (see Non-Patent Document 1). It has been reported that the expression is directly induced by estrogen receptor α (ERα), and GREB1 is highly expressed in ERα-positive breast cancer cells, but is not expressed in ERα-negative cells. It has been reported that ERα binds to a promoter region of GREB1 gene to induce expression of GREB1, and its direct interaction with ERα activates the transcriptional activity (see Non-Patent Document 2). Actually, it has been reported that the expression level of GREB1 mRNA is correlated with an ERα expression level in a breast cancer (see Non-Patent Document 2). Moreover, it is known that knockdown of GREB1 suppresses proliferation of breast cancer cells, and overexpression of GREB1 accelerates proliferation of breast cancer cells (see Non-Patent Document 3). Furthermore, it has been reported that GREB1 is not expressed in ERα-negative cells, and that GREB1 is expressed in ERα-positive ovarian cancer cells and plays an important role in ovarian cancer cell proliferation (see Non-Patent Document 4). Because a GREB1 promoter region has androgen-responsive sequences, GREB1 is induced by androgen in androgen receptor (AR)-positive prostate cancer cells, but is not induced in AR-negative cells (see Non-Patent Document 5). Thus, GREB1 acts as a mediator for proliferation of sex hormone-sensitive tumors.

However, it has still not been elucidated whether or not the expression of GREB1 plays a role in tumorigenesis of tumors other than sex hormone-sensitive tumors.

### PRIOR ART DOCUMENTS

### Non-Patent Documents

Non-Patent Document 1: Cancer Res. November 15; 60(22): 6367-75.
Non-Patent Document 2: Cell Rep. 2013 Feb. 21; 3(2): 342-9.
Non-Patent Document 3: Breast Cancer Res Treat. 2005 July; 92(2): 141-9.
Non-Patent Document 4: Int J Cancer. 2014 Sep. 1; 135(5): 1072-84.
Non-Patent Document 5: Prostate. 2006 Jun. 1; 66(8): 886-94.

### SUMMARY OF THE INVENTION

### Problems to be Solved by the Invention

An object of the present invention is to provide a novel antitumor agent. Further, other objects of the present invention are to provide a testing method for estimating whether or not a subject is affected with hepatoblastoma, hepatocellular cancer, malignant melanoma, and neuroblastoma.

### Means for Solving the Problem

The present inventors have made extensive study to solve the above-described problems. As a result, the present inventors have found that, in hepatoblastoma cells, liver cancer cells, malignant melanoma cells, neuroblastoma cells, or the like, although cell proliferation is not accelerated by sex hormones such as estrogen and androgen, there are tumor cells expressing GREB1, and found that GREB1 participates in proliferation of the tumor cells having such characteristics (i.e., GREB1-positive tumors that do not show sex hormone sensitivity). Specifically, the present inventors have found that, among the GREB1-positive tumors that do not show sex hormone sensitivity, in hepatoblastoma and hepatocellular cancer, GREB1 is a target gene for Wnt/β-catenin signal, in malignant melanoma, GREB1 is a target gene for a transcriptional factor MITF (Microphthalmia-associated transcription factor), and in neuroblastoma, GREB1 may contribute to gene amplification. Further, the present inventors have found that proliferation of the sex hormone insensitive GREB1-positive tumors is suppressed by siRNAs or antisense oligonucleotides against GREB1. In addition, the present inventors have also found tumor tissue-specific GREB1 expression in hepatoblastoma, hepatocellular cancer, malignant melanoma, and neuroblastoma by immunochemical investigation. The present invention has been made by further investigations based on these findings.

That is, the present invention provides the following aspects of the invention.

Item 1. A therapeutic agent for a sex hormone insensitive GREB1-positive tumor, containing a substance that suppresses expression of GREB1 as an active ingredient.

Item 2. The therapeutic agent according to item 1, wherein the substance is at least one nucleic acid drug selected from the group consisting of siRNA, shRNA, dsRNA, an antisense nucleic acid, and ribozyme against GREB1.

Item 3. The therapeutic agent according to items 1 or 2, wherein the tumor is hepatoblastoma, hepatocellular cancer, malignant melanoma, or neuroblastoma.

Item 4. A testing method for estimating whether or not a subject is affected with hepatoblastoma, hepatocellular cancer, malignant melanoma, or neuroblastoma, the method including a step of measuring expression level of GREB1 in a liver tissue, a skin tissue, or a nerve tissue sampled from the subject.

Item 5. The testing method according to item 4, wherein the expression level of GREB1 in a liver tissue, a skin tissue, or a nerve tissue is measured by immunohistochemical analysis using an anti-GREB1 antibody.

Item 6. A testing agent for hepatoblastoma, containing an agent for detecting GREB1.

Item 7. The testing agent according to item 6, wherein the agent for detecting GREB1 is an anti-GREB1 antibody or a fragment thereof, or a primer capable of hybridizing to GREB1 mRNA or GREB1 cDNA.

Item 8. A therapeutic method for treating a sex hormone insensitive GREB1-positive tumor, comprising administering a therapeutically effective amount of a substance that suppresses expression of GREB1 to a patient affected with the sex hormone insensitive GREB1-positive tumor.

Item 9. The therapeutic method according to item 9, wherein the tumor is hepatoblastoma, hepatocellular cancer, malignant melanoma, or neuroblastoma.

Item 10. Use of a substance that suppresses expression of GREB1 for the manufacture of a therapeutic agent for a sex hormone insensitive GREB1-positive tumor.

Item 11. The use according to item 10, wherein the tumor is hepatoblastoma, hepatocellular cancer, malignant melanoma, or neuroblastoma.

Item 12. A substance that suppresses expression of GREB1 for use in the treatment of a sex hormone insensitive GREB1-positive tumor.

Item 13. The substance that suppresses expression of GREB1 according to item 12, wherein the tumor is hepatoblastoma, hepatocellular cancer, malignant melanoma, or neuroblastoma.

### Advantages of the Invention

The therapeutic agent of the present invention has been developed based on the novel finding that GREB1 is a downstream target gene of Wnt/β-catenin signal in sex hormone insensitive GREB1-positive tumors, and can effectively suppress proliferation of the tumor cells by suppressing expression of GREB1 as a target molecule. Examples of the sex hormone insensitive GREB1-positive tumors include hepatoblastoma, hepatocellular cancer, malignant melanoma, neuroblastoma, and the like. For example, hepatoblastoma is a child-specific disease and a rare cancer, the occurrence of which is infrequent, and therefore advances in development of a chemotherapeutic agent or a molecular target drug have been previously insufficient. However, one embodiment of the present invention makes it possible to provide an excellent molecular target drug for hepatoblastoma. Further, analysis using gene expression database has revealed that expression of GREB1 is extremely limited in other major normal tissues. Accordingly, the therapeutic agent of the present invention against GREB1 as a molecular target is also innovative in little possible side effects.

Further, the testing method of the present invention can use the expression level of GREB1 in a biopsy specimen or a surgically excised specimen suspected of a neoplastic disease as an indicator, and therefor can be applied to an early diagnosis of hepatoblastoma, hepatocellular cancer, malignant melanoma, or neuroblastoma, and, in the case of a surgically excised specimen, can be applied to assessment of the region of histological progression of these tumors.

### BRIEF DESCRIPTION OF THE DRAWINGS

FIG. 1a shows figures showing mRNA expression analysis of target genes located downstream of Wnt/β-catenin signaling using hepatoblastoma cells. FIG. 1b shows an illustration depicting a TCF4 binding sequence located upstream (−443 to −448) of human GREB1 gene, and an image showing the results obtained by immunoprecipitating chromatin derived from HepG2 cells using predetermined antibodies, and analyzing the TCF4 binding site (−443 to −448) by PCR using domain-specific primers. FIG. 1c shows a graph (on the left) showing the results obtained by measuring expression level of GREB1 mRNA in HepG2 cells transfected with control siRNA or siRNA against β-catenin by real-time PCR analysis, and images (on the right) showing the results obtained by probing the cell lysates with an anti-GREB1 antibody, an anti-Axin2 antibody, an anti-β-catenin antibody, and an anti-HSP90 antibody. FIG. 1d shows images showing the results obtained by immunostaining hepatoblastoma tissues (n=11) with an anti-GREB1 antibody and hematoxylin, and a graph showing proportions of three categories having percentages of anti-GREB1-immunostained areas of <5%, 5 to 30%, and 30 to 95% with respect to the total area of a neoplastic lesion. FIG. 1e shows images showing the results obtained by immunostaining specimens of a hepatoblastoma tissue with an anti-GREB1 antibody or an anti-β-catenin antibody, and hematoxylin, in which boxes made in solid lines represent a region containing densely stained β-catenin, whereas boxes made in broken lines represent a region containing lightly stained β-catenin. FIG. 1f shows a diagram showing the result obtained, using 55 cases in the mRNA profile dataset of hepatoblastoma (GEO ID: gse75271), by analyzing GREB1 mRNA expression levels in 50 neoplastic lesion areas and 5 nonneoplastic areas. FIG. 1g shows diagrams each showing the result obtained, using 55 cases in the mRNA profile dataset of hepatoblastoma (GEO ID: gse75271), by analyzing the correlation between the expression levels of target genes in the Wnt/β-catenin pathway (Y axis) and the expression levels of GREB1 gene (X axis).

FIG. 2a shows a graph showing the results obtained by treating HepG2 cells with an estrogen receptor antagonist ICI-182,786, and thereafter measuring expression level of GREB1 mRNA by real-time PCR. FIG. 2b shows graphs showing the results obtained by treating BMEL cells with a GSK3 inhibitor CHIR99021 (Wnt/β-catenin pathway activator) at a final concentration of 5 μM, and thereafter measuring expression level of GREB1 mRNA by real-time PCR. FIG. 2c shows graphs showing the results obtained by introducing siRNA against β-catenin into Huh6 cells, and thereafter measuring expression levels of GREB1 mRNA and β-catenin mRNA by real-time PCR. FIG. 2d shows a graph showing the results obtained by measuring expression levels of GREB1 mRNA in HepG2 cells and Huh6 cells by real-time PCR. FIG. 2e shows graphs showing the results obtained by treating MCF7 cells with estrogen receptor antagonist ICI-182,786, CHIR99021, or a combination thereof, and thereafter measuring expression levels of GREB1 and Axin2 mRNA by real-time PCR. FIG. 2f shows graphs showing the results obtained by treating HLE, SNU387, SNU449, and Huh7 cells, which are hepatocellular cancer cell lines, with CHIR99021 at a final concentration of 5 μM, and thereafter measuring expression levels of GREB1 mRNA by real-time PCR. FIG. 2g shows images showing the results obtained by immunostaining hepatoblastoma tissues (n=11) with an anti-β-catenin antibody and hematoxylin, and a graph showing proportions of three categories having percentages of β-catenin-immunostained areas of <5%, 5 to 30%, and 30 to 95% with respect to the total area of a neoplastic lesion. FIG. 2h shows images showing the results obtained by immunostaining a specimen of a hepatoblastoma tissue with an anti-GREB1 antibody and hematoxylin, in which boxes made in solid lines represent a region containing densely stained GREB1 (solid and unpolarized), whereas boxes made in broken lines represent a region containing lightly stained GREB1 (tubular and polarized). FIG. 2i shows the results each obtained, using 55 cases in the mRNA profile dataset of hepatoblastoma (GEO ID: gse75271), by analyzing the correlation between the expression levels of a target gene in the estrogen signaling pathway (Y axis) and the expression levels of GREB1 gene (X axis). FIG. 2j shows diagrams showing the results obtained, using 55 cases in the mRNA profile dataset of hepatoblastoma (GEO ID: gse75271), by analyzing expression levels of GREB1mRNA, GS mRNA, or LGR5 mRNA in cases in which exons 3 and 4 of the β-catenin gene were wild type (n=3), or exons 3 and 4 had a point mutation (n=19) or a deletion (n=28).

FIG. 3a shows a graph showing the results obtained by transfecting HepG2 cells or HepG2 cells allowed to express GREB1 (HepG2/GREB1) with control siRNA or siRNA against β-catenin, performing two-dimensional culture (plastic dish culture), and counting the cell number over time. FIG. 3b shows images showing the results obtained by transfecting HepG2 cells with control siRNA or two different siRNAs (GREB1 #1 siRNA and GREB1 #2 siRNA), and probing the cell lysates with an anti-GREB1 antibody, an anti-Axin2 antibody, an anti-β-catenin antibody, and an anti-HSP90 antibody. FIG. 3c shows graphs showing the results obtained by analyzing GREB1 mRNA level, PRLR mRNA level, and XBP1 mRNA level in HepG2 cells transfected with control siRNA or siRNA (GREB1 #2 siRNA) by real-time PCR. FIG. 3d shows the results each obtained, using 55 cases in the mRNA profile dataset of hepatoblastoma (GEO ID: gse75271), by analyzing the correlation between the expression levels of a hepatoblastoma-specific marker gene (Y axis) and the expression levels of GREB1 gene (X axis). FIG. 3e shows a graph showing the results obtained by performing two-dimensional culture (plastic dish culture) of the Huh6 cells allowed to express GFP or GFP-GREB1, and counting the cell number over time. FIG. 3f shows figures showing the results obtained by performing three-dimensional culture (culture using Matrigel) of mock Huh6 cells or GREB1-expressing Huh6 cells, and measuring the size of formed spheres. FIG. 3g shows images showing the results obtained by probing cell lysates of HepG2 cells, GREB1 knockout HepG2 cells (GREB1 KO HepG2), or GREB1 KO HepG2 cells allowed to express GFP-GREB1 (GREB1 KO HepG2/GREB1) with an anti-GREB1 antibody, an anti-GFP antibody, and an anti-histone H3 antibody. FIG. 3h shows a graph showing the results obtained by performing two-dimensional culture (plastic dish culture) of HepG2 cells (Control), GREB1 knockout HepG2 cells (GREB1 KO), or GREB1 KO HepG2 cells allowed to express GREB1 (GREB1 KO/GREB1), and counting the cell number over time. FIG. 3i shows graphs showing the results obtained by analyzing DLK1 mRNA level and AFP mRNA level in HepG2 cells, GREB1 knockout HepG2 cells (GREB1 KO), or GREB1 KO HepG2 cells allowed to express GREB1 (GREB1 KO/GREB1) by real-time PCR. FIG. 3j shows images showing the results obtained by probing cell lysates of HepG2 cells, GREB1 knockout HepG2 cells (GREB1 KO), or GREB1 KO HepG2 cells allowed to express GREB1 (GREB1 KO/GREB1) with an anti-cyclin A antibody, an anti-phosphorylated Histone H3 (pHistone H3) antibody, and an anti-histone H3 antibody.

FIG. 4a shows a graph showing the results obtained by transfecting mock HepG2 cells or GREB1-expressing HepG2 cells with control siRNA or two different types of siRNA against GREB1 (GREB1 #1 siRNA and GREB1 #2 siRNA), performing two-dimensional culture (culture using plastic dish), and counting the cell number over time. FIG. 4b shows figures showing the results obtained by culturing HepG2 cells transfected with control siRNA or siRNA (GREB1 #2 siRNA) in three-dimensional Matrigel for 5 days, staining the cells with phalloidin, and measuring areas of spheres and calculating the proportions of polarized spheres having lumens. FIG. 4c shows images showing the results obtained by measuring expression levels of GREB1 mRNA, DLK1 mRNA, AFP mRNA, and PEG3 mRNA in HepG2 cells transfected with control siRNA or siRNA against GREB1 (GREB1 #2 siRNA) by real-time PCR analysis. FIG. 4d shows images showing the results obtained by culturing HepG2 cells transfected with control siRNA or two different types of siRNA against GREB1 (GREB1 #1 siRNA and GREB1 #2 siRNA) in a medium containing 0.1% FBS for 1 day, and probing the cell lysates with an anti-cyclinA antibody, an anti-cyclin B antibody, an anti-phosphorylated histone H3 antibody, an anti-histone H3 antibody, an anti-GREB1 antibody, and an anti-HSP90 antibody. FIG. 4e shows diagrams showing the results each obtained, using 55 cases in the mRNA profile dataset of hepatoblastoma (GEO ID: gse75271), by analyzing the correlation between the expression levels of a cell proliferation marker gene (Y axis) and the expression levels of GREB1 gene (X axis). FIG. 4f shows figures showing the results obtained from HepG2 cells transfected with control siRNA or two different types of siRNA against GREB1 (GREB1 #1 siRNA and GREB1 #2 siRNA), culturing in media containing 0.1% FBS (caspase inhibitor Z-VAD was contained in one condition, and not contained in the other condition) for 2 days, staining with propidium iodide (PI) and Hoechst33342, and calculating the cell viability, FIG. 4g shows images showing the results obtained from HepG2 cells transfected with control siRNA or two different types of siRNA against GREB1 (GREB1 #1 siRNA and GREB1 #2 siRNA), culturing for 2 days, and probing the cell lysates with an anti-cleaved caspase 3 antibody, an anti-PARP1 antibody, and an anti-HSP90 antibody.

FIG. 5a shows illustrations of wild type GREB1 and a GREB1ΔNLS mutant, and images showing the results obtained by staining X293T cells expressing the HA-FLAG-GREB1ΔNLS mutant with an anti-FLAG antibody, phalloidin, and Hoechst33342. FIG. 5b shows images showing the results obtained by probing cell lysates (Input) and anti-GFP-antibody immunoprecipitates (IP) of X293T cells expressing HA-FLAG-GREB1 wild type X293T or HA-FLAG-GREB1ΔNLS mutant, and expressing GFP, GFP-Smad3, GFP-Smad4, or GFP-Smad7 with an anti-HA antibody or an anti-GFP antibody. FIG. 5c shows images showing the results obtained by probing cell lysates (input) and anti-Smad2/3-antibody immunoprecipitates (IP) of HepG2 cells with an anti-Smad2/3 antibody and an anti-GREB1 antibody. FIG. 5d shows illustrations of Smad2 and Smad2 mutants (N and C), and images showing the results obtained by probing lysates (Input) and anti-GFP-antibody immunoprecipitates (IP) of X293T cells expressing HA-FLAG-mGREB1, and GFP or GFP-Smad2 (Full, mutant N, or mutant C) with an anti-FLAG antibody or an anti-GFP antibody. FIG. 5e shows images showing the results obtained by precipitating HepG2 cell lysates using recombinant GST, recombinant GST-Smad2/MH1, or recombinant GST-Smad2/MH2, and probing the cell lysates (Input) and glutathione-sepharose precipitates (pulldown) by CBB staining, or with an anti-GREB1 antibody and an anti-Smad4 antibody. FIG. 5f shows illustrations of GREB1 mutants (N: 1 to 666, M: NLS/667-1333, and C: NLS/1334-1954), and images showing the results obtained by staining X293T cells expressing GFP-GREB1 mutants with an anti-GFP antibody and Hoechst33342. FIG. 5g shows images showing the results obtained by probing lysates (Input) and anti-GFP-antibody immunoprecipitates (IP) of X293T cells expressing FLAG-Smad3, and GFP, GFP-GREB1, or a GFP-GREB1 mutant with an anti-FLAG antibody or an anti-GFP antibody.

FIG. 6a shows images showing the results obtained by staining X293T cells expressing GFP-Smad3, GFP-Smad4, or GFP-Smad7 with an anti-GFP antibody and Hoechst33342. FIG. 6b shows images showing the results obtained by probing cell lysates (Input) and anti-Smad2/3-antibody immunoprecipitates (IP) of Huh6 cells allowed to express GFP-GREB1 with an anti-Smad2/3 antibody and an anti-GFP antibody. FIG. 6c shows images showing the results obtained by probing cell lysates (Input) and anti-GFP-antibody immunoprecipitates (IP) of X293T expressing GFP, GFP-Smad3, GFP-Smad4, or GFP-Smad7 with an anti-β-catenin antibody, an anti-c-Myc antibody, or an anti-GFP antibody. FIG. 6d shows images showing the results obtained by precipitating lysates of Huh6 cells allowed to express GFP-GREB1 using recombinant GST, recombinant GST-Smad2/MH1, or recombinant GST-Smad2/MH2, and probing cell lysates (Input) and glutathione sepharose precipitates (pulldown) by CBB staining, or with an anti-GFP antibody. FIG. 6e shows illustrations of GREB1 mutants (NSL/667-195 (ΔN), Δ667-1333 (ΔM), NSL/1-133 (ΔC)), and images showing the results obtained by probing lysates (Input) and GFP-antibody immunoprecipitates (IP) of X293T cells expressing FLAG-Smad3, and GFP, GFP-GREB1, or GFP-GREB1 mutants (NSL/667-195 (ΔN), Δ667-1333 (ΔM), NSL/1-1333 (ΔC)) with an anti-FLAG antibody or an anti-GFP antibody.

FIG. 7a shows diagrams showing the result obtained, using 55 cases in the mRNA profile dataset of hepatoblastoma (GEO ID: gse75271), by analyzing PAI-1 mRNA and GADD45B mRNA expression levels in 50 neoplastic lesion areas and 5 nonneoplastic areas. FIG. 7b shows diagrams each showing the result obtained, using 55 cases in the mRNA profile dataset of hepatoblastoma (GEO ID: gse75271), by analyzing the correlation between the expression levels of TGFβ signal target genes (Y axis) and the expression levels of GREB1 gene (X axis). FIG. 7c shows graphs showing the results obtained by transfecting HepG2 cells expressing GFP (HepG2/GFP), or HepG2 cells expressing GFP-GREB1 (HepG2/GFP-GREB1) with control siRNA or GREB1 #2 siRNA, and measuring mRNA levels of PAI-1 and SNAIL2 in the cells. FIG. 7d shows graphs showing the results obtained by transfecting HepG2 cells with control siRNA or GREB1 #2 siRNA, culturing in the presence or absence of a TGFβ receptor inhibitor (ALK5 inhibitor), and measuring mRNA levels of PAI-1 and SNAIL2 in the cells. FIG. 7e shows images showing the results obtained by probing lysates (Input) and anti-Smad2/3-antibody immunoprecipitates (IP) of HepG2 cells transfected with control siRNA or GREB1 #2 siRNA with an anti-p300 antibody, an anti-GREB1 antibody, or an anti-Smad2/3 antibody. FIG. 7f shows figures showing the results obtained by probing lysates (Input) and GFP-antibody immunoprecipitates (IP) of X293T cells expressing HA-FLAG-GREB1, GFP-Smad2 mutant (C), or GFP with an anti-p300 antibody, an anti-FLAG antibody, or an anti-GFP antibody. FIG. 7g shows images showing the results obtained by culturing HepG2 cells transfected with control siRNA or GREB1 #2 siRNA in the presence or absence of TGFβ, and analyzing PAI-1 contained in cell lysates (Input) and immunoprecipitates (IP) of the cultured cells for the exon 2 domain by PCR using a domain-specific primer. FIG. 7h shows graphs showing the results obtained by transfecting HepG2 cells with a plasmid denoted by the title of each graph, and measuring expression levels of SNAIL2 mRNA, p15 mRNA, or Axin2 mRNA by real-time PCR. FIG. 7i shows a graph showing the results obtained by performing two-dimensional culture of HepG2 cells transfected with control siRNA, TGFβ1 siRNA, GREB1 siRNA (GREB1 #2 siRNA), or TGFβ1 siRNA and GREB1 siRNA (GREB1 #2 siRNA) in combination, and counting the cell number over time. FIG. 7j shows a graph showing the results obtained by transfecting HepG2 cells and Smad2/3 knockout HepG2 cells (HepG2/Smad2/3 KO) with control siRNA or GREB1 siRNA (GREB1 #2 siRNA), performing two-dimensional culture, and counting the cell number over time.

FIG. 8a shows diagrams showing the results obtained, using 55 cases in the mRNA profile dataset of hepatoblastoma (GEO ID: gse75271), by analyzing Axin2 mRNA and DKK1 mRNA expression levels in 50 neoplastic lesion areas and 5 nonneoplastic areas. FIG. 8b shows a graph showing the results obtained by analyzing PAI-1 mRNA levels in HepG2 cells (Control), GREB1 knockout HepG2 cells (GREB1 KO), or GREB1 KO HepG2 cells allowed to express GREB1 (GREB1 KO/GREB1) by real-time PCR. FIG. 8c shows images showing the results obtained by culturing HepG2 cells transfected with control siRNA or siRNA against GREB1 (GREB1 #2 siRNA) in the presence or absence of 10 ng/mL of TGFβ for 30 minutes, and thereafter staining the cells with an anti-Smad2/3 antibody and Hoechst33342. FIG. 8d shows images showing the results obtained by culturing HepG2 cells transfected with control siRNA or siRNA against GREB1 (GREB1 #2 siRNA) in the presence or absence of TGFβ, and probing cell lysates (Input) and anti-Smad2/3-antibody immunoprecipitates (IP) with an anti-Smad4 antibody or an anti-Smad2/3 antibody. FIG. 8e shows images showing the results obtained by culturing HepG2 cells transfected with control siRNA or siRNA against GREB1 (GREB1 #2 siRNA) in the presence or absence of TGFβ, and probing cell lysates with an anti-GREB1 antibody, an anti-phosphorylated Smad2/3 (pSmad2/3) antibody, or an anti-Smad2/3 antibody. FIG. 8f shows graphs showing the results obtained by allowing HepG2 cells to express a TGFBR1/T204D mutant, and analyzing AFP mRNA and DLK1 mRNA levels by real-time PCR. FIG. 8g shows images showing the results obtained by probing lysates of HepG2 cells and Smad2/3 knockout HepG2 cells (HepG2/Smad2/3 KO) with an anti-Smad2/3 antibody or an anti-HSP90 antibody. FIG. 8h shows graphs showing the results obtained by transfecting HepG2 cells and Smad2/3 knockout HepG2 cells (HepG2/Smad2/3 KO) with control siRNA or siRNA against GREB1 (GREB1 #2 siRNA), and analyzing AFP mRNA levels by real-time PCR.

FIG. 9a shows a graph showing the results obtained by analyzing gene expression levels of TGFβ1, TGFβ2, or TGFβ3 using RNA sequence data of HepG2 in the mRNA profile dataset of Cancer Cell Line Encyclopedia (CCLE). FIG. 9b shows graphs showing the results obtained by analyzing PAI-1 mRNA levels, TGFB1 mRNA levels, and GREB1 mRNA levels in HepG2 cells transfected with control siRNA, TGFβ1 siRNA, GREB1 siRNA (GREB1 #2 siRNA), or siRNA of TGFβ1 and siRNA of GREB1 (GREB1 #2 siRNA) in combination by real-time PCR. FIG. 9c shows images showing the results obtained by probing lysates of HepG2 cells transfected with control siRNA, TGFβ1 siRNA, GREB1 siRNA (GREB1 #2 siRNA), or siRNA of TGFβ1 and siRNA of GREB1 (GREB1 #2 siRNA) in combination with an anti-GREB1 antibody, an anti-TGFB1 antibody, or an anti-HSP90 antibody. FIG. 9d shows a graph showing the results obtained by culturing HepG2 cells transfected with control siRNA, TGFβ1 siRNA, or TGFβ1 siRNA and GREB1 siRNA (GREB1 #2 siRNA) in combination in the presence or absence of 0.01, 0.1, or 1 ng/ml of TGFβ for 4 hours, and thereafter analyzing PAI-1 mRNA levels by real-time PCR. FIG. 9e shows graphs showing the results obtained by analyzing p15 mRNA levels, p21 mRNA levels, and p27 mRNA levels in HepG2 cells transfected with control siRNA or siRNA against GREB1 (GREB1 #2 siRNA) by real-time PCR. FIG. 9f shows graphs showing the results obtained by transfecting HepG2 cells and Smad2/3 knockout HepG2 cells (HepG2/Smad2/3 KO) with control siRNA or siRNA against GREB1 (GREB1 #2 siRNA), and analyzing p15 mRNA levels by real-time PCR. FIG. 9g shows a graph showing the results obtained by performing two-dimensional culture (culture using plastic dish) of HepG2 cells transfected with control siRNA, GREB1 siRNA (GREB1 #2 siRNA), p15 siRNA, or GREB1 siRNA (GREB1 #2 siRNA) and p15 siRNA in combination, and counting the cell number over time. FIG. 9h shows images showing the results obtained by culturing HepG2 cells transfected with control siRNA, GREB1 siRNA (GREB1 #2 siRNA), p15 siRNA, or GREB1 siRNA (GREB1 #2 siRNA) and p15 siRNA in combination in media containing 0.1% FBS (caspase inhibitor Z-VAD was contained in one condition, and not contained in the other condition) for 2 days, staining with propidium iodide (PI) and Hoechst33342, and calculating the cell viability. FIG. 9i shows graphs showing the results obtained by treating MCF7 cells with an estrogen receptor antagonist ICI-182,786, and thereafter measuring PAI-1 mRNA and GREB1 mRNA expression levels by real-time PCR. FIG. 9j shows graphs showing the results obtained by transfecting MCF7 cells with control siRNA or siRNA against GREB1 (GREB1 #2 siRNA), and analyzing PAI-1 mRNA or GREB1 mRNA levels by real-time PCR. FIG. 9k shows images showing the results obtained by probing cell lysates (input) and anti-Smad2/3-antibody immunoprecipitates (IP) of MCF7 cells with an anti-Smad2/3 antibody and an anti-GREB1 antibody.

FIG. 10a shows an image showing the result obtained by fixing HepG2 cells expressing GFP-GREB1, and staining with an anti-GFP antibody and Hoechst33342. FIG. 10b shows images showing the results obtained by fixing HepG2 cells expressing GFP-GREB1, and staining with an anti-GFP antibody, an anti-Fibrillarin antibody, an anti-SC35 antibody, an anti-PML antibody, or an anti-Coilin antibody, and Hoechst33342. FIG. 10c shows images showing the results obtained by fixing HepG2 cells expressing GFP-GREB1 and FLAG-SMAD3, and staining with an anti-GFP antibody, an anti-FLAG antibody, and Hoechst33342. FIG. 10d shows images showing the results obtained by incubating HepG2 cells in the presence of ethynyl uridine (EU) for 30 minutes, and thereafter fixing the cells and observing the fixed cells.

FIG. 11a shows figures showing the results obtained by culturing HepG2 cells in the presence or absence of 10 ng/mL of TGFβ for 30 minutes, fixing the cultured cells, and staining with an anti-SMAD2/3 antibody, an anti-GREB antibody, and Hoechst33342. FIG. 11b shows images showing the results obtained by culturing HepG2 cells in the presence or absence of TGFβ, fixing the cultured cells, and staining with an anti-GREB1 antibody, an anti-phosphorylated SMAD2/3(pSMAD2/3) antibody, and Hoechst33342. FIG. 11c shows images showing the results obtained by culturing HepG2 cells in the presence or absence of TGFβ, fixing the cultured cells, reacting with a mouse anti-GREB1 antibody and a rabbit anti-SMAD2/3 antibody, and further binding with secondary antibodies (PLA probe) (on the left), and a graph showing proportions of cells having PLA signal spots in the nucleus (on the right). FIG. 11d shows images showing the results obtained by staining X293T cells expressing GFP-fused GREB1 mutants (N: 1 to 666, M: NLS/667-1333, and C: NLS/1334-1954) with an anti-GFP antibody. FIG. 11e shows figures showing the results obtained by incubating HepG2 cells expressing GFP-SMAD3 with or without expression of HA-FLAG-GREB1 in the presence of ethynyl uridine (EU), fixing the incubated cells, and staining the fixed cells with an anti-GFP antibody, an anti-GREB1 antibody, and Hoechst33342.

FIG. 12a shows images showing the results obtained by immunostaining hepatoblastoma tissues (n=11) with an anti-β-catenin antibody and hematoxylin, and a graph showing proportions of three categories having percentages of anti-β-catenin-immunostained areas of <5%, 5 to 30%, and 30 to 95% with respect to the total area of a neoplastic lesion. FIG. 12b shows images showing the results obtained by staining tissue sections of livers resected from mice transduced with ΔN90 β-catenin and YAPS127A (BY model), ΔN90 β-catenin and c-Met(BM) model, or ΔN90 β-catenin, YAPS127A, and c-Met (BYM model) with an anti-GREB1 antibody or an anti-DLK1 antibody, and hematoxylin, and the results obtained by observing the livers. FIG. 12c shows graphs showing the results obtained by extracting total RNAs from liver tumor nodules of mice transduced with ΔN90 β-catenin and YAPS127A (BY model), ΔN90 β-catenin and c-Met (BM model), or ΔN90 β-catenin, YAPS127A, and c-Met (BYM model) (BY: n=3; BM: n=7; BYM: n=11), and analyzing mRNA levels of GREB1 and TASCSTD1 by real-time PCR.

FIG. 13a shows images showing the results obtained by fixing Huh6 cells or HepG2 cells, and staining the fixed cells with an anti-YAP antibody and Hoechst33342. FIG. 13b shows graphs showing the results obtained by measuring expression levels of GREB1 mRNA, YAP mRNA, and TAZ mRNA in HepG2 cells or Huh6 cells transfected with control siRNA or siRNA against YAP/TAZ by real-time PCR. FIG. 13c shows graphs showing the results obtained by treating Huh6 cells with CHIR99021, XMU-MP1 as an Mst1/2 kinase inhibitor (YAP activator), or a combination thereof, and thereafter measuring expression levels of GREB1 mRNA, Axin2 mRNA, and Cyr61 mRNA by real-time PCR. FIG. 13d shows graphs showing the results obtained by measuring expression levels of c-Met mRNA, Axin2 mRNA, GREB1 mRNA, ANKRD1 mRNA, and Cyr61 mRNA in HepG2 cells transfected with control siRNA or siRNA against c-Met by real-time PCR. FIG. 13e shows images showing the results obtained by probing lysates of HepG2 cells transfected with control siRNA or siRNA against c-Met with an anti-c-Met antibody, an anti-phosphorylated β-catenin (pY654) antibody, an anti-β-catenin antibody, an anti-Axin2 antibody, an anti-GREB1 antibody, and an anti-β-actin antibody.

FIG. 14a shows images showing the results obtained by observing livers of mice transduced with ΔN90 β-catenin, YAPS127A, and c-Met (BYM, control; C1 to C7), livers of mice dosed with ΔN90 β-catenin, YAPS127A, and c-Met together with GREB1 shRNA (BYM+ GREB1 shRNA; BYM GREB1 KD mice, K1 to K6), and a liver of an untreated mouse (NL mouse). FIG. 14b shows images showing the results obtained by sampling 6 tumor nodules from each of the above-described BYM mice (C1 to C7), and measuring expression levels of hepatoblastoma-related genes. FIG. 14c shows images showing the results obtained by staining tissue sections of livers resected from BYM mice in a GREB1 high group (C4) and a GREB1 low group (C3) with hematoxylin-eosin. FIG. 14d shows images showing the results obtained by staining tissue sections of livers resected from the BYM mice in a GREB1 high group (C4) and a GREB1 low group (C3) with an anti-GREB1 antibody or an anti-DLK1 antibody, and hematoxylin. FIG. 14e shows graphs showing the ratios of the weight of the liver obtained from each of the mice to the whole body weight, and the results obtained by measuring serum AFP levels of each mouse. FIG. 14f shows graphs showing the results obtained by extracting total RNAs from tumor nodules of 3 BYM mice (GREB1 high groups: C1, C4, and C6), 4 BYM mice (GREB1 low groups: C2, C3, C5, and C7), and 2 BYM mice dosed with GREB1 shRNA (BYM GREB1 KD mice; K2 and K4), and analyzing mRNA levels of GREB1, DLK1, and TACSTD1 by real-time PCR. FIG. 14g shows images showing the results obtained by staining tissue sections of livers of BYM mice (GREB1 high group: C4) and BYM mice dosed with GREB1 shRNA (GREB1 shRNA: K2) with hematoxylin-eosin or an anti-GREB1 antibody, and hematoxylin. FIG. 14h shows images showing the results obtained by staining tissue sections of livers of BYM mice (GREB1 high group: C4) and BYM mice dosed with GREB1 shRNA (GREB1 shRNA: K2) with an anti-N-cadherin antibody and Hoechst33342 (on the left), and a graph showing the results obtained by calculating the ratios of N-cadherin expression levels in a neoplastic lesion area to N-cadherin expression levels in a nonneoplastic area (lower graph).

FIG. 15a shows graphs showing the results obtained by sampling tumor nodules from livers of mice transduced with ΔN90 β-catenin, YAPS127A, and c-Met (BYM, control; C1 to C7), and a liver of an untreated mouse (NL mouse), and measuring expression levels of the transduced ΔN90 β-catenin, YAPS127A, and c-Met. FIG. 15b shows a graph showing the results obtained by extracting total RNAs from a liver of an untreated mouse (NL), tumor nodules (n=42) of mice transduced with ΔN90 β-catenin, YAPS127A, and c-Met (BYM), and nonneoplastic tissues (n=8) of the BYM mice, and measuring mRNA levels of GREB1 by real-time PCR. FIG. 15c shows graphs showing the results obtained by using tumor nodules sampled from the above-described BYM mice (C1 to C7), analyzing the correlation between the expression levels of DLK1 mRNA, TACSTD1 mRNA, GPC3 mRNA, MEG3 mRNA, and Axin2 mRNA (Y axis) and the expression levels of GREB1 gene (X axis). FIG. 15d shows images showing the results obtained by staining tissue sections of livers resected from BYM mice of a GREB1 high group (C4) and a GREB1 low group (C3) with hematoxylin-eosin. FIG. 15e shows images showing the results obtained by probing lysates of HepG2 cells (Control), GREB1 knockout HepG2 cells (GREB1 KO), or HepG2 cells knocked out by GREB1 and Smad2/3 in combination (GREB1 KO+Smad2/3 KO) with an anti-GREB1 antibody, an anti-Smad2/3 antibody, and an anti-HSP90 antibody. FIG. 15f shows a graph showing the results obtained by performing two-dimensional culture (plastic dish culture) of HepG2 cells (WT), GREB1 knockout HepG2 cells (GREB1 KO), or HepG2 cells knocked out by GREB1 and Smad2/3 in combination (GREB1 KO/Smad2/3 KO), and counting the cell number over time.

FIG. 16a shows figures showing the results obtained by grafting wild type HepG2 cells (control), GREB1 knockout HepG2 cells (GREB1 KO), cells that were GREB1 knockout HepG2 cells allowed to express GREB1 GREB1KO/GREB1), cells that were GREB1 knockout HepG2 cells allowed to express GREB1ΔNLS (GREB1KO/GREB1ΔNLS), or cells in which GREB1 and Smad2/3 were knocked out in combination (GREB1 KO/Smad2/3 KO) into mice subcutaneously, and observing the appearance and measuring the weights of the xenograft tumors 28 days after grafting. FIG. 16b shows figures showing the results obtained by transfecting HepG2 cells expressing GFP or GFP-GREB1 with control ASO or each GREB1 ASO, culturing in three-dimensional Matrigel, staining the cultured cells with phalloidin and Hoechst33342, and calculating the areas of spheres. FIG. 16c shows figures showing the results obtained by grafting Matrigel containing HepG2 cells into livers of nude mice, injecting control ASO, GREB1 ASO-6921, or GREB1 ASO-7724 subcutaneously twice every week starting 3 days after grafting, and observing the appearance of the liver tumors and measuring the tumor weights at day 27 post grafting. FIG. 16d shows figures showing the results obtained by staining sections of tumor of each of the above-described livers with an anti-Ki-67 antibody and hematoxylin, and calculating the ratios of the number of Ki-67-positive cells to the number of hematoxylin-positive cells (total cells). FIG. 16e shows figures showing the results obtained by staining sections of the tumors of each of the above-described livers with an anti-cleaved-caspase 3 antibody and hematoxylin, and calculating the ratios of the number of cleaved-caspase 3-positive cells to the number of hematoxylin-positive cells (total cells). FIG. 16f shows graphs showing the results obtained by analyzing mRNA levels of GREB1 and PAI-1 in tumors of each of the above-described livers by real-time PCR.

FIG. 17a shows images showing the results obtained by transfecting HepG2 cells with control ASO or each GREB1 ASO, and probing the cell lysates with an anti-GREB1 antibody and an anti-HSP90 antibody. FIG. 17b shows images showing the results obtained by grafting Matrigel containing HepG2 cells into livers of nude mice, injecting control ASO, GREB1 ASO-6921, or GREB1 ASO-7724 subcutaneously twice every week starting 3 days after grafting, and at day 27 post grafting, using a nonneoplastic part of the livers as samples, staining sections of the samples with an anti-cleaved caspase 3 antibody and hematoxylin and calculating the ratios of cleaved caspase 3-positive cells. FIG. 17c shows figures showing the results obtained by introducing ΔN90 β-catenin, YAPS127A, and c-Met (BYM) into mice, injecting control ASO and ASO targeting mouse GREB1 (mGREB1 ASO-5715) subcutaneously twice every week starting 3 days after grafting, and observing the appearance of the liver tumors and calculating the ratio of weight of each liver to the whole body weight 6 to 7 weeks after grafting. FIG. 17d shows a graph showing the results obtained by analyzing GREB1 mRNA levels in tumors of each of the above-described livers by real-time PCR.

FIG. 18A shows a scatter diagram showing GREB1 mRNA expression levels in cancer cell lines obtained from The Cancer Cell Line Encyclopedia (CCLE) using DepMap portal. FIG. 18B shows images showing the results obtained by probing each cancer cell lysate with an anti-GREB1 antibody and an anti-GAPDH antibody. FIG. 18C shows an image showing the results obtained by immunostaining neuroblastoma tissue specimens (n=13) with an anti-GREB1 antibody, an anti-f-catenin antibody, and hematoxylin, and a graph showing proportions of three categories having percentages of GREB1- or β-catenin-immunostained areas of <5%, 5 to 30%, and 30 to 95% with respect to the total area of a neoplastic lesion. FIG. 18D shows a table showing the results obtained by analyzing the relationships between the amount of GREB1 protein and the amount of β-catenin protein based on the immunohistological analysis in FIG. 18C. FIG. 18E shows a graph showing the results obtained by performing two-dimensional culture (plastic dish culture) of CHP212 cells transfected with control ASO or GREB1 ASO, and counting the cell number (average±SD) over time.

FIG. 19A shows images showing the results obtained by immunostaining specimens of hepatocellular cancer tissues (n=210) with an anti-GREB1 antibody, an anti-β-catenin antibody, and hematoxylin. FIG. 19B shows a table showing the results obtained by analyzing the relationships between the amount of GREB1 protein and the amount of β-catenin protein based on the immunohistological analysis in FIG. 19A. FIG. 19C shows images showing the results obtained by probing each cancer cell lysate with an anti-GREB1 antibody and an anti-GAPDH antibody. FIG. 19D shows images showing the results obtained by probing lysates each cells transfected with control siRNA or β-catenin siRNA with an anti-GREB1 antibody, an anti-β-catenin antibody, and an anti-GAPDH antibody. FIG. 19E shows graphs showing the results obtained by performing two-dimensional culture (plastic dish culture) of Hep3B cells or JHH7 cells transfected with control siRNA or GREB1 #2 siRNA, and counting the cell number over time. FIG. 19F shows the results obtained by grafting Hep3B cells (Control KO) and GREB1 knockout Hep3B cells (GREB1 KO) into nude mice (n=6) subcutaneously, and images on the left show typical appearances of xenograft tumors excised 45 days after grafting, a graph on the center shows changes in tumor sizes over time, and a graph on the right shows the results obtained by measuring tumor weights 45 days after grafting. FIG. 19G shows the results obtained by grafting JHH7 cells (Control KO) and GREB1 knockout JHH7 cells (GREB1 KO) into nude mice (n=6) subcutaneously, and images on the left show typical appearances of xenograft tumors excised 14 days after grafting, a graph on the center shows changes in tumor sizes over time, and a graph on the right shows the results obtained by measuring tumor weights 14 days after grafting. **P<0.01; *P<0.05.

FIG. 20A shows images showing the results obtained by probing each cell lysate with an anti-GREB1 antibody and an anti-HSP90 antibody. FIG. 20B shows an illustration on the left of isoforms of human GREB1 (in the illustration, as is an abbreviation for amino acid). FIG. 20B shows diagrams on the right showing average exon expression levels (OPKM; Observations PER Kilobases of exon/splice per Million aligned reads) of human GREB1 gene in breast cancers and cutaneous malignant melanomas obtained in the TCGA dataset using TCGA SpliceSeq (http://projects.insilico.us.com/TCGASpliceSeq). FIG. 20C shows a table showing a list of top 10 genes that have correlation with GREB1 expression in the dataset of cutaneous malignant melanomas obtained in the TCGA using ‘R2: genomics analysis and visualization platform (http://r2.amc.nl)’. R denotes Pearson correlation coefficient, * denotes known downstream target genes of MITF. FIG. 20D shows images showing the results obtained by immunostaining specimens of cutaneous malignant melanomas with an anti-GREB1 antibody, an anti-MITF antibody, and hematoxylin. FIG. 20E shows images showing the results obtained by probing CLO679 cells (control) and MITF knockout CLO679 cells (MITF KO) with an anti-GREB1 antibody, an anti-MITF antibody, and an anti-clathrin antibody. FIG. 20F shows a graph showing the results obtained by performing two-dimensional culture (plastic dish culture) of SKMEL28 cells transfected with control siRNA or GREB1 siRNA, and counting the cell number (average±SD) over time. FIG. 20G shows a graph showing the results obtained by performing two-dimensional culture (plastic dish culture) of SKMEL28 cells transfected with control ASO or GREB1 ASO, and counting the cell numbers (average±SD) over time.

### EMBODIMENTS OF THE INVENTION

### 1. Therapeutic Agent for Hormone Insensitive GREB1-Positive Tumor

The therapeutic agent of the present invention is a drug for use in the treatment of a sex hormone insensitive GREB1-positive tumor, characterized in that a substance that suppresses expression of GREB1 is used as an active ingredient. The therapeutic agent of the present invention will be described in detail below.

### [Active Ingredient]

In the therapeutic agent of the present invention, a substance that suppresses expression of GREB1 is used as an active ingredient. GREB1 acts as a target gene of Wnt/β-catenin signal and MITF in sex hormone insensitive GREB1-positive tumor cells, sometimes undergoes gene amplification, and has an effect of accelerating proliferation of the tumor cells. The therapeutic agent of the present invention can suppress expression of GREB1, and thereby effectively prevent proliferation of the tumor.

The amino acid sequence and base sequence of GREB1 are also publicly known. For example, the amino acid sequence of human GREB1 (isoform a) is known as shown in SEQ ID NO: 1, the base sequence of mRNA of human GREB1 (isoform a) is known as shown in SEQ ID NO: 2, and the base sequence of cDNA encoding human GREB1 (isoform a) is known as shown in SEQ ID NO: 3.

In the present invention, a “substance that suppresses expression of GREB1” is not particularly limited as long as the substance is pharmacologically acceptable and can suppress expression of GREB1 from DNA (GREB1 gene) encoding GREB1. The substance that suppresses expression of GREB1 may be any substance that exerts the effect of suppressing expression of GREB1 in any stage such as transcription, posttranscriptional regulation, translation, and posttranslational modification of GREB1 gene. The substance that suppresses expression of GREB1 include, specifically, nucleic acid drugs including nucleic acid molecules that suppress transcription of GREB1 gene, such as decoy nucleic acids; RNA molecules or precursors thereof that have RNA interference effect on mRNA of GREB1, such as siRNAs, shRNAs, and dsRNAs; nucleic acid molecules that suppress translation of mRNA of GREB1, such as miRNAs, antisense nucleic acids (antisense DNAs, antisense RNAs), and ribozymes. These nucleic acid molecules may be used alone, or in combination of two or more. The base sequences of these nucleic acid molecules may be suitably designed based on information of the base sequence of GREB1 gene according to publicly-known techniques by persons skilled in the art.

Among these nucleic acid molecules, from the viewpoint of ease of clinical application or the like, preferred examples include siRNAs, shRNAs, dsRNAs, antisense nucleic acids, and ribozymes, with siRNAs, shRNAs, and antisense nucleic acids being more preferred.

Further, the nucleic acid molecules may have various modifications that are generally applied to nucleic acids to give resistance to nuclease or the like, if necessary. Examples of such modification include modifications to a sugar chain moiety, such as 2′-fluorination and 2′-O-methylation; modifications to a base moiety; modifications to a phosphate moiety, such as amination, lower alkyl amination, acetylation, and phosphorothioate. Further, the nucleic acid molecules may have artificial nucleic acids (bridged nucleic acids, peptide nucleic acids, locked nucleic acids, or the like) that are introduced into a part of an RNA molecule and/or a DNA molecule.

For example, preferred examples of the bridged nucleic acid include a nucleotide having a structure represented by the following general formula (1).

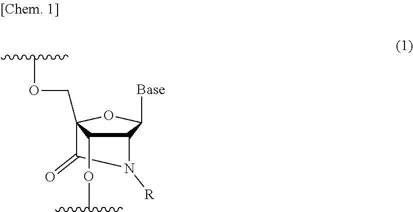

In general formula (1), Base represents a base specified in a base sequence, and specifically represents an optionally substituted purine-9-yl group or 2-oxo-1,2-dihydropyrimidine-1-yl group. Examples of the substituent include, specifically, a hydroxy group, a C1-6 linear alkyl group, a C1-6 linear alkoxy group, a mercapto group, a C1-6 linear alkylthio group, an amino group, a C1-6 linear alkylamino group, and a halogen atom.

Further Base in general formula (1) include, specifically, when the base is A (adenine), a 6-aminopurine-9-yl group optionally substituted with a substituent; when the base is G (guanine), a 2-amino-6-hydroxypurine-9-yl group optionally substituted with a substituent; when the base is C (cytosine), a 2-oxo-4-amino-1,2-dihydropyrimidin-1-yl group optionally substituted with a substituent (e.g., including a 4-amino-5-methyl-2-oxo-1,2-dihydropyrimidine-1-yl group, a 5-methylcytosine-1-yl group, a 2′-O-methylcytosine-1-yl group, and the like); when the base is T (thymine), a 2-oxo-4-hydroxy-5-methyl-1,2-dihydropyrimidine-1-yl group optionally substituted with a substituent.

In general formula (1), R represents a hydrogen atom, a C1-7 alkyl group which may be branched or may form a ring, a C2-7 alkenyl group which may be branched or may form a ring, a C3-12 aryl group which may have a substituent and may have a heteroatom, and an aralkyl group having a C3-12 aryl moiety which may have a substituent and may have a heteroatom. Examples of the substituent which may be contained in the aryl group or aralkyl group include, specifically, a hydroxy group, a C1-6 linear alkyl group, a C1-6 linear alkoxy group, a mercapto group, a C1-6 linear alkylthio group, an amino group, a C1-6 linear alkylamino group, and a halogen atom. Examples of R preferably include a hydrogen atom, a methyl group, an ethyl group, an n-propyl group, an isopropyl group, a phenyl group, or a benzyl group, more preferably include a hydrogen atom or a methyl group, and particularly preferably include a methyl group. In the present specification, 2′,4′-bridged nucleotide that is represented by general formula (1) in which R is a methyl group is sometimes denoted by “AmNA”.

Further, other examples of the bridged nucleotide include a nucleotide having a structure represented by the following general formula (2). This nucleotide is a 2′,4′-bridged nucleotide that is publicly known and also referred to as a guanidine bridged nucleic acid (WO 2014/046212).

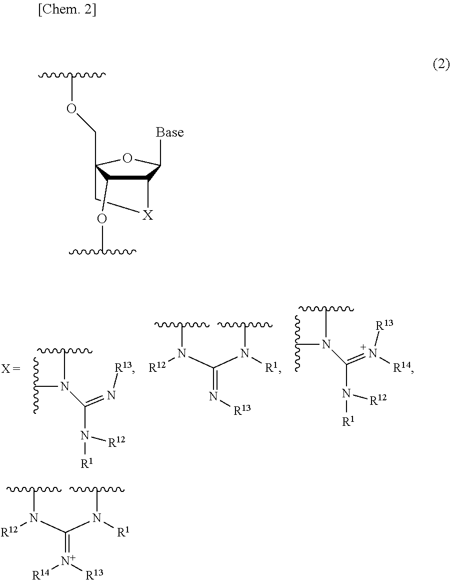

In general formula (2), Base is the same as the Base in the above-described general formula (1). In general formula (2), R¹, R¹², and R¹³ may be the same or different, and each represent a hydrogen atom or a C1-7 alkyl group which may be branched or may form a ring, and R¹⁴ represents a hydrogen atom.

Further, other examples of the bridged nucleotide include a nucleotide having a structure represented by the following general formula (3). This nucleotide is a 2′,4′-bridged nucleotide that is publicly known and also referred to as a guanidine bridged nucleic acid (WO 2015/125783).

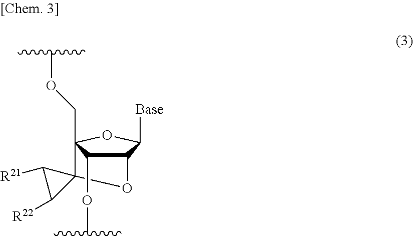

In general formula (3), Base is the same as the Base in the above-described general formula (1). Further, in general formula (3), R²¹ and R²² may be the same or different, and each represent a hydrogen atom; a C1-7 alkyl group which may be substituted with a C3-12 aryl group optionally containing a heteroatom and may be branched or may form a ring; an aralkyl group having a C3-12 aryl moiety optionally containing a heteroatom; or R²¹ and R²² together may form a group —(CH₂)ₙ—, wherein n is an integer of 2 to 5.

Further, other examples of the bridged nucleotide include a nucleotide having a structure represented by the following general formula (4) or (4′). This nucleotide is a 2′,4′-bridged nucleotide that is publicly known and also referred to as an ethyleneoxy-bridged nucleic acid (WO 2016/017422).

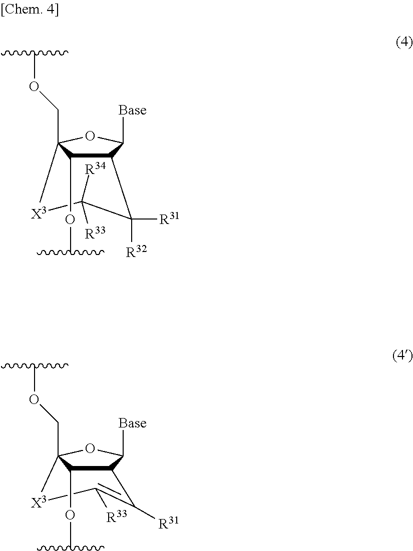

In general formulae (4) and (4′), Base is the same as the Base in the above-described general formula (1). Further, in general formulae (4) and (4′), X³ represents an oxygen atom or a sulfur atom.

In general formulae (4) and (4′), R³¹ and R³² may be the same or different, and each represent a hydrogen atom; a hydroxy group; a C1-7 alkyl group which may be branched or may form a ring; a C1-7 alkoxy group which may be branched or may form a ring; or an amino group. Further, in the case of general formula (4), R³¹ and R³² together may form a group ═C(R³⁵)R³⁶, wherein R³⁵ and R³⁶ may be the same or different, and each represent a hydrogen atom, a hydroxy group, a mercapto group, an amino group, a C1-6 linear or branched alkoxy group, a C1-6 linear or branched alkylthio group, a C1-6 cyanoalkoxy group, or a C1-6 linear or branched alkyl amino group.

In general formulae (4) and (4′), R³³ represents a hydrogen atom, a C1-7 alkyl group which may be branched or may form a ring, a C1-7 alkoxy group which may be branched or may form a ring, or a C1-6 linear or branched alkylthio group.

In general formula (4), R³⁴ represents a hydrogen atom, a C1-7 alkyl group which may be branched or may form a ring, a C1-7 alkoxy group which may be branched or may form a ring, or a C1-6 linear or branched alkylthio group.

Further, other examples of the bridged nucleotide include those having the structures shown below. Base and R in the following structural formulae are the same as Base and R in the above-described general formula (1).

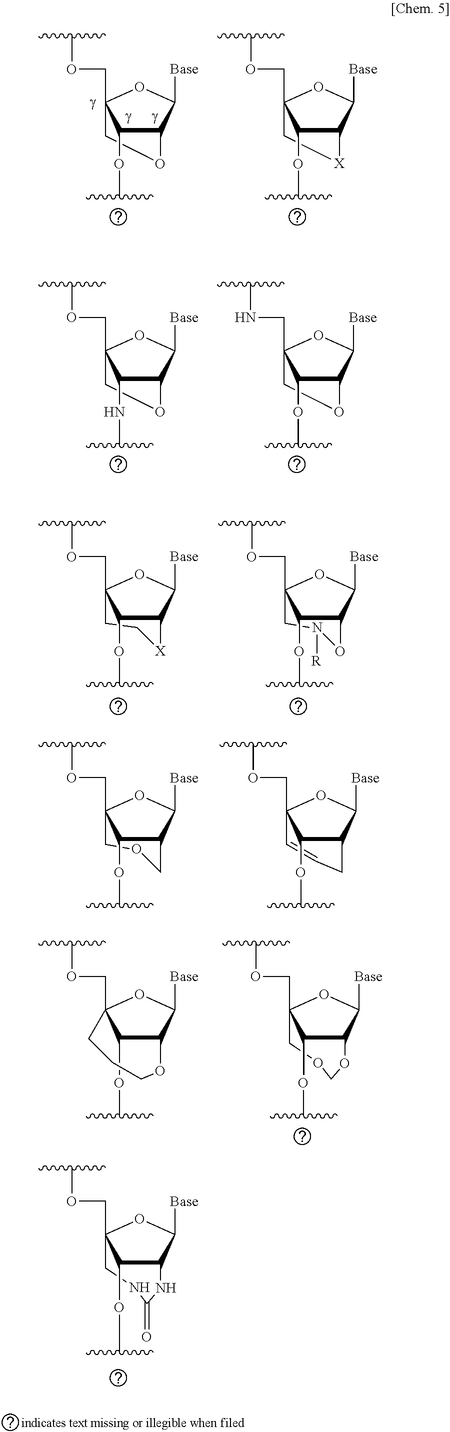

In the nucleic acid molecule used in the present invention, chemical modifications may be applied to some nucleotides and/or linking moieties between nucleosides, or may be applied to all nucleotides and/or linking moieties between nucleosides.

When the nucleic acid molecule used in the present invention is an antisense nucleic acid, in preferred examples of the chemical modification, the nucleic acid molecule contains, for example, at least one, preferably one to ten, more preferably two to eight, still more preferably two to six, and particularly preferably five 2′,4′-bridged nucleotides. Further, in preferred examples of the nucleic acid molecule that contains a 2′,4′-bridged nucleotide, for example, first to third nucleotides from 5′ end and second and third nucleotides from 3′ end are 2′,4′-bridged nucleotides (preferably AmNAs).

Further, in preferred examples of the chemical modification that applied to the nucleic acid molecule used in the present invention, at least one linking moiety between nucleosides is a phosphorothioate bond. Further, in preferred examples of the nucleic acid molecule that contains a phosphorothioate bond, with respect to the total linking moieties between nucleosides as 100%, for example, preferably 50% or more, more preferably 80% or more, still more preferably 90% or more, and particularly preferably 100% (all linking moieties between nucleosides) of the linking moieties are phosphorothioate bonds.

When the nucleic acid molecule used in the present invention is a siRNA against human GREB1, specific examples of the siRNA include a siRNA containing a sense strand consisting of a base sequence represented by SEQ ID NO: 4 and an antisense strand consisting of a base sequence represented by SEQ ID NO: 5; and a siRNA containing a sense strand consisting of a base sequence represented by SEQ ID NO: 6 and an antisense strand consisting of a base sequence represented by SEQ ID NO: 7. Herein, in SEQ ID NOs: 4 to 6, bases at positions 1 to 19 constitute an RNA chain, and bases at positions 20 and 21 are overhangs (deoxythymidines).

Further, when the nucleic acid molecule used in the present invention is an antisense nucleic acid (an antisense DNA, ASO) against human GREB1, specific examples of the ASO include chemically modified ASOs consisting of the following sequences A to D.

5(Y)\hat{ }G(Y)\hat{ }A(Y)\hat{ }a\hat{ }t\hat{ }g\hat{ }g\hat{ }c\hat{ }a\hat{ }g\hat{ }g\hat{ }a\hat{ }5(Y)\hat{ }A(Y)\hat{ }g (sequence A: used as ASO-6434 in Examples)

G(Y)\hat{ }T(Y)\hat{ }5(Y)\hat{ }t\hat{ }g\hat{ }t\hat{ }t\hat{ }t\hat{ }c\hat{ }a\hat{ }a\hat{ }g\hat{ }T(Y)\hat{ }A(Y)\hat{ }a (sequence B: used as ASO-6921 in Examples)

T(Y)\hat{ }5(Y)\hat{ }T(Y)\hat{ }a\hat{ }g\hat{ }t\hat{ }t\hat{ }c\hat{ }t\hat{ }c\hat{ }a\hat{ }t\hat{ }5(Y)\hat{ }A(Y)\hat{ }a (sequence C: used as ASO-6968 in Examples)

A(Y)\hat{ }T(Y)\hat{ }T(Y)\hat{ }g\hat{ }a\hat{ }g\hat{ }g\hat{ }g\hat{ }t\hat{ }a\hat{ }g\hat{ }g\hat{ }5(Y)\hat{ }A(Y)\hat{ }a (sequence D: used as ASO-7724 in Examples)

In the above-described sequences A to D, “G(Y)” represents guanine having a structure of AmNA (a 2′,4′-bridged nucleotide that is represented by the above-described general formula (1) in which R is a methyl group), “A(Y)” represents adenine having a structure of AmNA, “T(Y)” represents thymine having a structure of AmNA, “5(Y)” represents 5-methylcytosine having a structure of AmNA, and lowercase “a, t, c, g” each represent an unmodified DNA, and “\hat{ }” represents a phosphorothioate bond.

Further, when the nucleic acid molecule used in the present invention is a shRNA against mouse Greb1, specific examples of DNA encoding the shRNA include a base sequence represented by SEQ ID NO: 8.

Further, when the nucleic acid molecule used in the present invention is an RNA molecule, it may be designed such that the RNA molecule can be produced in vivo. Specifically, it is possible that DNA encoding the RNA molecule that is introduced into an expression vector for mammalian cells. Examples of the expression vector include virus vectors such as retrovirus vectors, lentivirus vectors, adenovirus vectors, adeno-associated virus vectors, herpesvirus vectors, and Sendai virus vectors, and animal cell expression plasmids.

### [Target Disease]

GREB1 is specifically expressed in sex hormone insensitive GREB1-positive tumor cells, and accelerates proliferation of the tumor cells. Thus, a therapeutic agent of the present invention can control proliferation of the tumor cells by suppressing expression of GREB1. Accordingly, the therapeutic agent of the present invention is used in the treatment of hormone insensitive GREB1-positive tumors.

In the present invention, the GREB1-positive tumor refers to a tumor formed of tumor cells in which expression of GREB1 occurs. Whether a tumor is a GREB1-positive tumor or not can be determined by performing immunohistochemical analysis on a sampled neoplastic lesion tissue. Specifically, when a sampled neoplastic lesion tissue is immunostained using an anti-GREB1 antibody and a region in which expression of GREB1 is observed accounts for 5% or more of the neoplastic lesion area, the tumor is determined to be GREB1-positive. Preferred examples of the tumor as a target for the treatment with a therapeutic agent of the present invention preferably include a tumor in which a region showing high expression of GREB1 accounts for 20% or more of the neoplastic lesion area, particularly preferably a tumor in which a region showing high expression of GREB1 accounts for 50% or more of the neoplastic lesion area.

Also, whether a tumor is a GREB1-positive tumor or not can be determined by collecting RNA from a sampled tumor tissue, and measuring by quantitative PCR. In this case, the determination of whether or not GREB1 is expressed can be made by using a nonneoplastic region of the same tissue in the same case from which the tumor is sampled as a standard. Specifically, when the amount of GREB1 in the cell lysate of a tumor tissue is larger than the amount of GREB1 in the cell lysate of a nonneoplastic region of the same tissue in the same case from which the tumor is sampled, the tumor is determined to be GREB1-positive.

Further, in the present invention, the sex hormone insensitive GREB1-positive tumor (GREB1-positive tumor that does not show hormone sensitivity) refers to a tumor formed of GREB1-positive tumor cells that do not have a “property in which expression of GREB1 is facilitated via a hormone receptor by stimulation with a sex hormone such as estrogen or androgen and proliferation is accelerated”. Examples of sex hormone sensitive GREB1-positive tumors include, for example, breast cancer and ovarian cancer as tumors proliferation of which is accelerated by estrogen, and, prostate cancer as a tumor proliferation of which is accelerated by androgen. These tumors are excluded from the target of the treatment in the present invention.

In the present invention, examples of the sex hormone insensitive GREB1-positive tumor as a target for the treatment include, specifically, hepatoblastoma, hepatocellular cancer, malignant melanoma (melanoma), neuroblastoma, small cell lung cancer.

Further, the therapeutic agent of the present invention can be used for not only human, but also for mammals such as cattle, pig, dog, cat, goat, rat, mouse, rabbit, and the like. However, the therapeutic agent is preferably used as a pharmaceutical for human.

### [Form of Administration]

Forms of administration of the therapeutic agent of the present invention may be either oral administration or parenteral administration as long as an antitumor effect can be obtained, and may be suitably chosen depending on the type of active ingredient used. Specifically, examples of the form of administration of the therapeutic agent of the present invention include parenteral administration such as injection administration (such as intravenous injection, subcutaneous injection, intramuscular injection, intraperitoneal injection, local injection into an affected part), and suppository administration.

The therapeutically effective amount of dosage of the therapeutic agent of the present invention may be suitably chosen depending on the type of active ingredient used, the form of administration, extent of progression of the sex hormone insensitive GREB1-positive tumor to be treated, or the like. For example, when a nucleic acid molecule is used as an active ingredient, the nucleic acid molecule may generally be administered in a dose of about 0.1 mg/kg body weight to 100 mg/kg body weight per administration at a frequency of once every 3 to 7 days.

The therapeutic agent of the present invention may be used alone, or may be used in combination with one or two or more other medicines and/or radiation therapies having an antitumor effect.

### [Form of Formulation]

The therapeutic agent of the present invention is formulated into a form of formulation according to the type of active ingredient and the form of administration. Examples of the form of formulation of the antitumor agent of the present invention include liquid formulations such as liquid preparation, suspension, emulsion, and injectable preparation.

Further, the therapeutic agent of the present invention is formulated by adding pharmacologically acceptable carriers or additives depending on the form of formulation. For example, in the case of a liquid formulation, the formulation can be made by using physiological saline, a buffer, or the like.

Further, in the therapeutic agent of the present invention, when a nucleic acid molecule is used as an active ingredient, it is desirable that the formulation is made together with a nucleic acid transfection enhancer so that the nucleic acid molecule is easily transported into tumor cells. Examples of the nucleic acid transfection enhancer include, specifically, Lipofectamine, Oligofectamine, RNAiFect, liposome, polyamines, DEAE-dextran, calcium phosphate, dendrimers.

### 2. Testing Method for Hepatoblastoma, Hepatocellular Cancer, Malignant Melanoma, or Neuroblastoma

The testing method of the present invention is a testing method for estimating whether or not a subject is affected with hepatoblastoma, hepatocellular cancer, malignant melanoma, or neuroblastoma, characterized by including a step of measuring expression level of GREB1 in a liver tissue, a skin tissue, or a nerve tissue sampled from the subject. The present invention makes it possible, by using expression level of GREB1 in a liver tissue, a skin tissue, or a nerve tissue sampled from a subject as an indicator, to diagnose whether or not the subject is affected with hepatoblastoma, hepatocellular cancer, malignant melanoma, or neuroblastoma.

When hepatoblastoma or hepatocellular cancer is a target for the test, expression level of GREB1 in a liver tissue sampled from a subject with suspected neoplastic liver disease may be measured. Since hepatoblastoma is a malignant tumor that occurs in the liver of a child, examples of the target subject of the test for hepatoblastoma include a child with suspected neoplastic liver disease.

When malignant melanoma is a target for the test, expression level of GREB1 in a skin tissue sampled from a subject with suspected neoplastic skin disease may be measured.

When neuroblastoma is a target for the test, expression level of GREB1 in a nerve tissue sampled from a subject with suspected neoplastic neurological disease may be measured. Since neuroblastoma is a malignant tumor that usually occurs in children, in the testing method of the present invention, examples of the target subject of the test for neuroblastoma preferably include a child with suspected neoplastic neurological disease.

The expression level of GREB1 in each tumor tissue sampled from a subject can be measured and determined by performing immunohistochemical analysis on the sampled piece of tissue (piece of lesion tissue). Specifically, when immunohistochemical analysis is performed on a sampled piece of tumor tissue using an anti-GREB1 antibody, and a region in which expression of GREB1 is observed accounts for 5% or more of the suspected tumor area, it is determined that GREB1 is expressed. When a region in which expression of GREB1 is observed accounts for 20% or more of the suspected tumor area, it is determined that expression level of GREB1 is high. When a region in which expression of GREB1 is observed accounts for 50% or more of the suspected tumor area, it is determined that expression level of GREB1 is particularly high.

Also, the expression level of GREB1 in each tumor tissue sampled from a subject can be measured by collecting RNA from the piece of tissue (piece of lesion tissue), and using quantitative PCR. In this case, the determination of the expression level of GREB1 can be made by using a nonneoplastic region of the same tissue in the same case from which the tumor is sampled as a standard. Specifically, when the amount of GREB1 in the cell lysates of a suspected tumor region is larger than the amount of GREB1 in the cell lysates of the nonneoplastic region in the same case from which the suspected tumor is sampled, it is determined that GREB1 is expressed.

In the testing method of the present invention, when the expression level of GREB1 in each of the tumor tissues is higher, it is speculated that the possibility of the subject being affected with hepatoblastoma, hepatocellular cancer, malignant melanoma, or neuroblastoma is higher.

Further, the present invention provides an agent for detecting GREB1 as a testing reagent for carrying out the testing method.

Examples of the agent for detecting GREB1 include an anti-GREB1 antibody and fragments thereof. The anti-GREB1 antibody may be labeled with biotin, a fluorescent label, magnetic beads, or the like, if necessary.

In addition, other examples of the agent for detecting GREB1 include a primer capable of hybridizing to GREB1 cDNA or GREB1 mRNA. When such primer is used as an agent for detecting GREB1, the testing reagent of the present invention may contain, in addition to the primer, an agent required for performing PCR.

### Examples

The present invention will be described in detail based on experimental data below, but the present invention is not limited thereto.

Additionally, cells used in the following studies are sometimes represented in the form of, for example, “X-expressing cells”, or the like. The expression “X-expressing cells” refers to cells that have been transfected with protein X and transformed so as to express the protein X artificially. For example, “GFP-GREB1-expressing X293T cells” refers to X293T cells that have been transfected with protein GFP-GREB1 and transformed so as to overexpress GFP-GREB1.

In addition, proteins are sometimes represented in the form of, for example, “A-B” (e.g., GFP-GREB1, etc.) below. This expression refers to a protein in which peptide A is fused with peptide B.

Additionally, cells transfected with various proteins used in the following studies were produced according to publicly-known genetic engineering techniques.

### 1. Test Material and Method

### 1-1. Cells and Antibodies

HepG2 cells, CHP212 cells, and SKNDZ cells were purchased from American Type Culture Collection (ATCC, Manassas, Va., USA). MCF7 cells, HLE cells, Huh7 cells, HLF cells, NBTU110 cells, SKMEL28 cells, Mewo cells, G361 cells, and COLO679 cells were purchased from Japanese Collection of Research Bioresources (JCRB, Osaka, Japan). Further, Lenti-X™ 293T (X293T) cells were purchased from Takara Bio Inc. (Japan). Huh6 cells used were supplied by Dr. H. Okuyama (Osaka University, Japan). SNU387 cells, SNU449 cells, BMEL cells, and HepG2 cells used were supplied by Dr. T. Kodama (Osaka University, Japan).

HepG2 cells were grown in Eagle's minimum essential medium (EMEM) medium supplemented with 10% fetal bovine serum (FBS), nonessential amino acids and glutamax. HLE cells, Huh6 cells, X293T cells, Huh7 cells, SNU387 cells, SNU449 cells, and HLF cells were grown in Dulbecco's modified Eagle's medium (DMEM) supplemented with 10% FBS. MCF7 cells were grown in DMEM supplemented with 10% FBS, nonessential amino acids, and 1 mM sodium pyruvate. BMEL cells were grown in DMEM/F12 medium supplemented with 10% FBS, 2.5 mM L-glutamine, 0.5 mM sodium pyruvate, 50 ng/ml EGF, 30 ng/ml IGF-II, and 10 μg/ml insulin. SKMEL28 cells, Mewo cells, and G361 cells were grown in EMEM supplemented with 10% FBS and nonessential amino acids. CHP212 cells and SKNDZ cells were grown in DMEM supplemented with 10% FBS and nonessential amino acids. OLO679 cells were grown in RPMI 1640 (RPMI) supplemented with 10% FBS and 2 mM L-glutamine.

Anti-GREB1 antibody (mouse monoclonal), anti-phosphorylated histone H3 (ser10) antibody, and anti-acetylated histone H4 antibody were purchased from Merck Millipore (Billerica, Mass., USA). Anti-HSP90 antibody, anti-cyclin A antibody, anti-cyclin B antibody, anti-Smad2/3 antibody (mouse monoclonal), anti-β-catenin antibody, and anti-clathrin antibody were purchased from BD Biosciences (San Jose, Calif., USA). Anti-GREB1 antibody (rabbit polyclonal), anti-Smad4 antibody, anti-p300 antibody, and anti-GFP antibody (mouse monoclonal) were purchased from Santa Cruz Biotechnology (Dallas, Tex., USA). Anti-Smad 2/3 antibody (rabbit monoclonal), anti-phosphorylated SMAD2 (Ser465/467)/SMAD3 (Ser423/425) antibody, anti-cleaved caspase 3 antibody, anti-PARP1 antibody, anti-Histone H3 antibody, anti-YAP1 antibody (rabbit monoclonal), anti-Ki67 antibody (rabbit monoclonal), anti-c-Myc antibody (rabbit monoclonal), anti-Smad2/3 (rabbit monoclonal for Western blotting), anti-Axin2 antibody, and anti-MITF antibody (for Western blotting) were purchased from Cell Signaling Technology (Beverly, Mass., USA). Anti-β-tubulin antibody, anti-β-actin antibody, anti-phosphorylated β-catenin (pTyr654) antibody, and anti-MITF antibody (for immunohistological staining) were purchased from Sigma-Aldrich (Steinheim, Germany). Anti-GFP antibody (rabbit polyclonal) was purchased from Life Technologies/Thermo Fisher Scientific (Carlsbad, Calif., USA). Anti-Smad2/3 antibody (rabbit monoclonal) was purchased from Abcam (Cambridge, UK). Anti-FLAG antibody and anti-GAPDH antibody were purchased from WAKO (Tokyo, Japan). Anti-mouse DLK1 antibody (rabbit monoclonal) was purchased from R&D Systems (Minneapolis, Minn., USA).

### 1-2. RNA Sequence Analysis

Libraries of HepG2 cells transfected with control siRNA or β-catenin siRNA were prepared by using TruSeq Stranded mRNA Sample Prep kit (Illumina, San Diego, Calif.). Sequence analysis was performed using Illumina HiSeq 2500 platform with 75-base single-end mode. Base call was performed using CASAVA 1.8.2 software (Illumina). Reads were mapped against human reference genome sequence (hg19) using TopHat v2.0.13 with Bowtie2 ver. 2.2.3 and SAMtools ver. 0.1.19 in combination for sequence determination. The number of fragments per kilobase of exon per million reads mapped (FPKMs) was calculated using Cuffnorm version 2.2.1.

From a total of 23,284 genes, 8,929 genes were extracted as genes having the normalized FPKM value of 3.0 or more. In β-catenin knockdown cells, 76 genes were downregulated 3-fold or more as compared to control cells (P<0.001 [Welch's t test]). GREB1-binding peaks were downloaded as TCF7L2#HepG2#hg19#1 geneset (465 genes) from ENCODE Transcription Factor Binding Site Profiles resource (http://amp.pharm.mssm.edu/Harmonizome/gene#set/TCF7L2#HepG2#hg19#1/ENCO DE+Transcription+Factor+Binding+Site+Profiles). Finally, 11 downregulated genes were found.

### 1-3. Open Source Clinical Data Analysis

Clinical data of cancer patients were analyzed and obtained using The Cancer Genome Atlas (TCGA) datasets available from web sites ‘depmap portal (https://depmap.org/portal/), TCGA SpliceSeq (http://projects.insilico.us.com/TCGASpliceSeq), and ‘R2: Genomics Analysis and Visualization Platform (http://r2.amc.nl)’. All gene expression data, P values, and r values were downloaded.

### 1-4. Patients and Cancer Tissues

Hepatoblastoma tissue (n=11) obtained from hepatoblastoma patients who underwent surgical treatment between January 2008 and March 2015 at Osaka University Hospital were used in this study. The patients were aged from 0 to 16 (median age of 3 years).

Further, neuroblastoma tissues (from 13 patients) and cutaneous malignant melanoma tissues (from 50 patients) obtained from patients who underwent surgical treatment at Osaka University Hospital, and hepatocellular cancer (HCC) tissues (stages I to IV) obtained from 210 patients who underwent surgical treatment at Kobe University Hospital were used in this study.

Resected specimens were visually observed, areas and sizes of tumors were measured, and the specimens were fixed with 10 vol % formalin and embedded to form paraffin embedded blocks which were subjected to histological analysis. The specimens used in this study were cut into 4 μm thick sections, stained with hematoxylin and eosin (H&E), immunoperoxidase, immuno-alkaline phosphatase, or the like, and subjected to each analysis.

The studies shown in the following sections 2-1 to 2-8 were carried out after receiving approval from the Medical Ethics Committee of Osaka University School of Medicine (No. 13552). Further, the studies shown in the following sections 2-9 to 2-11 were carried out in accordance with the Helsinki Declaration, and after receiving approval from the Medical Ethics Committee of Osaka University School of Medicine (No. 13455, 19292) and approval from the Medical Ethics Committee of Kobe University School of Medicine (No. B190073). All patients provided written informed consent.

### 1-5. Evaluation of Tumorigenesis by HTVi (Hydrodynamic Tail Vein Injection)

In the presence of Greb1 shRNA #3 (SEQ ID NO: 8) (20 μg) or control, and pLIVE-SB13 (8 μg), pT2BH-YAPS127A (20 μg), pT2BH-ΔN90 β-catenin-mutLuc3 (20 μg), pT3-EF1a-cMET (20 μg), and GFP2ALuc were diluted in 2.5 ml of physiological saline, and injected into tail veins of 8- to 9-week old male wild-type C57BL6/N mice within 5 to 7 seconds. Livers of the mice were collected at a predetermined time after injection (or at occurrence of the tumor), and various parameters associated with tumorigenesis were assessed. Specifically, this assessment was performed according to techniques described in Tao, J. et al., (2014). Gastroenterology 147, 690-701 and Tward, A. D. et al, (2007). Proc Natl Acad Sci USA 104, 14771-14776. In this evaluation, ΔN90 β-catenin refers to a β-catenin mutant in which amino acids at positions 1 to 90 of human ft-catenin were deleted, and YAPS127A refers to a YAP mutant in which serine at positon 127 of YAP was substituted by alanine.

### 1-6. Preparation of Antisense Oligonucleotide Against GREB1

Phosphorothioated I5-mer antisense oligonucleotides (ASOs) containing AmNA monomers were prepared (synthesized by GeneDesign (Ibaraki, Japan)). Sequences of the synthesized ASOs are as shown in Tables 8 and 9. In the present specification, “hGREB1-6424-AmNA (15)” is sometimes simply referred to as “ASO-6424” or “GREB1 ASO-6424”. Similar abbreviations are used for describing other ASOs. Using RNAiMAX (Invitrogen, Carlsbad, Calif., USA), HepG2 or COLO679 cells were transfected with 10 nM of ASOs. In experiments, the cells were used from 36 to 48 hours after transfection.

### 1-7. Xenograft Liver Tumorigenesis Assay by In Vivo ASO Treatment

Pellets of HepG2 cells (1×10⁷ cells) were suspended in 100 μl of Matrigel high concentration (Corning), and directly injected and grafted into the livers of 8-week old male BALB/cAJcl-nu/nu mice (nude mice; CLEA Japan) under anesthetic. From day 0, ASO (50 μg/body; about 2.5 mg/kg) was subcutaneously injected at a frequency of twice every two weeks. The mice were euthanized 27 days after grafting, and tumors were collected and subjected to histological analysis.

1-8. Knockdown of Protein by siRNA

In analysis using siRNAs, base sequences of siRNAs used are as shown in Table 1.

TABLE 1

| Target | Name | Sequence | |
|---|---|---|---|
| Control | Control | Sense | 5′- |
| | siRNA | strand | CAGUCGCGUUUGCGACUGGTT-3′ |
| | | | (SEQ ID NO: 9) |
| | | Antisense | 5′- |
| | | strand | CCAGUCGCAAACGCGACUGTT-3′ |
| | | | (SEQ ID NO: 10) |
| Human | GREB1 | Sense | 5′- |
| GREB1 | #1 | strand | GCCAUUCGUGUGCUUCCAUTT-3′ |
| | siRNA | | (SEQ ID NO: 4) |
| | | Antisense | 5′- |
| | | strand | AUGGAAGCACACGAAUGGCTT-3′ |
| | | | (SEQ ID NO: 5) |
| Human | GREB1 | Sense | 5′- |
| GREB1 | #2 | strand | CCUCCUACAAAGCAAUAUUTT-3′ |
| | siRNA | | (SEQ ID NO: 6) |
| | | Antisense | 5′- |
| | | strand | AAUAUUGCUUUGUAGGAGGTT-3′ |
| | | | (SEQ ID NO: 7) |
| Human | β- | Sense | 5′- |
| β- | catenin | strand | CCCACUAAUGUCCAGCGUUTT-3′ |
| catenin | #1 | | (SEQ ID NO: 11) |
| | siRNA | Antisense | 5′- |
| | | strand | AACGCUGGACAUUAGUGGGTT-3′ |
| | | | (SEQ ID NO: 12) |
| Human | β- | Sense | 5′- |
| β- | catenin | strand | GCAUAACCUUUCCCAUCAUTT-3′ |
| catenin | #2 | | (SEQ ID NO: 13) |
| | siRNA | Antisense | 5′- |
| | | strand | AUGAUGGGAAAGGUUAUGCTT-3′ |
| | | | (SEQ ID NO: 14) |
| Human | c-Met | Sense | 5′- |
| c-Met | siRNA | strand | GCAUCAGAACCAGAGGCUUTT-3′ |
| | | | (SEQ ID NO: 15) |
| | | Antisense | 5′- |
| | | strand | AAGCCUCUGGUUCUGAUGCTT-3′ |
| | | | (SEQ ID NO: 16) |
| Human | p15 | Sense | 5′- |
| p15 | siRNA | strand | GGAAUAACCUUCCAUACAUTT-3′ |
| | | | (SEQ ID NO: 17) |
| | | Antisense | 5′- |
| | | strand | AUGUAUGGAAGGUUAUUCCTT-3′ |
| | | | (SEQ ID NO: 18) |
| Human | YAP | Sense | 5′- |
| YAP | #1 | strand | GACAUCUUCUGGUCAGAGATT-3′ |
| | siRNA | | (SEQ ID NO: 19) |
| | | Antisense | 5′- |
| | | strand | UCUCUGACCAGAAGAUGUCTT-3′ |
| | | | (SEQ ID NO: 20) |
| Human | YAP | Sense | 5′- |
| YAP | #2 | strand | CUGGUCAGAGAUACUUCUUTT-3′ |
| | siRNA | | (SEQ ID NO: 21) |
| | | Antisense | 5′- |
| | | strand | AAGAAGUAUCUCUGACCAGTT-3′ |
| | | | (SEQ ID NO: 22) |
| Human | TAZ | Sense | 5′- |
| TAZ | #1 | strand | ACGUUGACUUAGGAACUUUTT-3′ |
| | siRNA | | (SEQ ID NO: 23) |
| | | Antisense | 5′- |
| | | strand | AAAGUUCCUAAGUCAACGUTT-3′ |
| | | | (SEQ ID NO: 24) |
| Human | TAZ | Sense | 5′- |
| TAZ | #2 | strand | AGGUACUUCCUCAAUCACATT-3′ |
| | siRNA | | (SEQ ID NO: 25) |
| | | Antisense | 5′- |
| | | strand | UGUGAUUGAGGAAGUACCUTT-3′ |
| | | | (SEQ ID NO: 26) |

In each base sequence of siRNA shown in the table, bases at position 1 to 19 constitute an RNA chain, and bases at positions 20 and 21 are overhangs
(deoxythymidines).

Using RNAiMAX (Life Technologies/Thermo Fisher Scientific), HepG2 cells, Huh6 cells, Hep3B cells, JHH7 cells, and SKMEL28 cells were each transfected with each siRNA (10 nm). In experiments, the cells were used from 48 to 120 hours after transfection.

### 1-9. Immunohistochemical Analysis

Immunohistochemical staining of tissue specimens were performed using DakoReal™ EnVision™ Detection System (Dako, Carpentaria, Calif., USA) and Warp Red™ Chromogen Kit (Biocare Medical, Concord, Calif., USA) according to protocols recommended by the manufacturers. Specifically, antigen retrieval of the tissue specimens was performed using decloaking chamber (Biocare Medical, Walnut Creek, Calif., USA). Next, endogenous peroxidase activities were blocked by 3% H₂O₂-methanol for 15 minutes, and then sections were incubated with goat serum for 1 hour to block nonspecific antibody binding sites. Thereafter, mouse anti-GREB1 antibody (1:100), mouse anti-β-catenin antibody (1:100), or rabbit anti-YAP1 antibody (1:100) were added to the tissue specimens, and the specimens were incubated at 4° C. for 16 hours, then incubated with horseradish peroxidase (HRP) labeled goat anti-mouse IgG or goat anti-rabbit IgG for 1 hour. Diaminobenzidine (DAB) (Dako) was used as a chromogen to visualize the mouse anti-GREB1 antibody, mouse anti-β-catenin antibody, or rabbit anti-YAP1 antibody bound to the tissue sections. Further, the tissue sections were counterstained with 0.1% (w/v) of hematoxylin. The regions stained with β-catenin, GREB1, and YAP were categorized into three levels (<5%, 5 to 30%, and 30 to 95%), and the tumor in which stained region (positive stain) was 5% or more with respect to the area of a neoplastic lesion was determined to be positive.

### 1-10. Immunofluorescent Staining

Cells grown on a glass cover slip were fixed with PBS containing 4% (w/v) paraformaldehyde at room temperature for 10 minutes, and permeabilization treatment was performed in PBS containing 0.2% (w/v) Triton X-100 and 2 mg/ml BSA for 10 minutes. Further, cells grown by three-dimensional culture were fixed in PBS containing 4% (w/v) paraformaldehyde at room temperature for 30 minutes, and blocked in PBS containing 0.5% (w/v) Triton X-100 and 40 mg/ml BSA for 30 minutes. The cells, which were fixed and permeabilization treated, were incubated with primary antibodies at room temperature for 3 hours or at 4° C. overnight, and further incubated with secondary antibodies according to protocols of the manufacturer (Molecular Probes, Carlsbad, Calif.) to prepare samples. Analysis of the samples were performed by observing the samples using LSM880 laser confocal microscope (Carl-Zeiss, Jena, Germany).

### 1-11. Visualization Using Heat Map of Gene Expression

Each value of gene expression level was normalized to GAPDH mRNA, and calculated as a ratio with respect to that of normal livers. These data were normalized using min-max normalization, and thereafter a colored heat map was made using Excel software (Microsoft, Redmond, Wash., USA).

### 1-12. Cell Proliferation Assay

Cells were seeded on a plate at 1.0×10⁴ cells/mL, and, after 12 hours, the medium was replaced with a medium containing 10% serum. While the cell number was counted every 48 hours, the cells were cultured up to 10 days. The measurement of the cell number was performed using Cyquant NF assays (Life Technologies/Thermo Fisher Scientific) according to protocols recommended by the manufacturer.

### 1-13. Quantitative RT-PCR

In quantitative RT-PCR, primers shown in Table 2 were used.

TABLE 2

| | | Sequence |
|---|---|---|
| Human | Forward | 5′-GTGCTCTACCGGCTCAAGTT-3′ |
| GREB1 | | (SEQ ID NO: 27) |
| | Reverse | 5′-ACCGAGTCCACCACGTAGAT-3′ |
| | | (SEQ ID NO: 28) |
| Human | Forward | 5′-CTGGCTCCAGAAGATCACAAAG- |
| Axin2 | | 3′ |
| | | (SEQ ID NO: 29) |
| | Reverse | 5′-CATCCTCCCAGATCTCCTCAAA- |
| | | 3′ |
| | | (SEQ ID NO: 30) |
| Human | Forward | 5′-TGGCTTCTCAGGCAATTTCT-3′ |
| DLK1 | | (SEQ ID NO: 31) |
| | Reverse | 5′-GGCTTGCACAGACACTCGTA-3′ |
| | | (SEQ ID NO: 32) |
| Human | Forward | 5′-AGCTTGGTGGTGGATGAAAC-3′ |
| AFP | | (SEQ ID NO: 33) |
| | Reverse | 5′-CCCTCTTCAGCAAAGCAGAC-3′ |
| | | (SEQ ID NO: 34) |
| Human | Forward | 5′-TCACTTCCAGCACAGCATTC-3′ |
| PEG3 | | (SEQ ID NO: 35) |
| | Reverse | 5′-CCTCAGCCAGTGTGGGTATT-3′ |
| | | (SEQ ID NO: 36) |
| Human | Forward | 5′-TCATGATGGTCAATGCCACT-3′ |
| PRLR | | (SEQ ID NO: 37) |
| | Reverse | 5′-GCGTGAACCAACCAGTTTTT-3′ |
| | | (SEQ ID NO: 38) |
| Human | Forward | 5′-TCACCCCTCCAGAACATCTC-3′ |
| XBP1 | | (SEQ ID NO: 39) |
| | Reverse | 5′-ACAGAGAAAGGGAGGCTGGT-3′ |
| | | (SEQ ID NO: 40) |
| Human | Forward | 5′-CACACCAGCTCCACTGAAGA-3′ |
| PAI-1 | | (SEQ ID NO: 41) |
| | Reverse | 5′-CTCCATCACAGGAGCAGACA-3′ |
| | | (SEQ ID NO: 42) |
| Human | Forward | 5′-CTTTTTCTTGCCCTCACTGC-3′ |
| SNAIL2 | | (SEQ ID NO: 43) |
| | Reverse | 5′-ACAGCAGCCAGATTCCTCAT-3′ |
| | | (SEQ ID NO: 44) |
| Human | Forward | 5′-CACGTGGAGCTGTACCAGAA-3′ |
| TGFβ1 | | (SEQ ID NO: 45) |
| | Reverse | 5′-TGCAGTGTGTTATCCCTGCT-3′ |
| | | (SEQ ID NO: 46) |
| Human | Forward | 5′-GACCGGGAATAACCTTCCAT-3′ |
| p15 | | (SEQ ID NO: 47) |
| | Reverse | 5′-CACCAGGTCCAGTCAAGGAT-3′ |
| | | (SEQ ID NO: 48) |
| Human | Forward | 5′-ATGAAATTCACCCCCTTTCC-3′ |
| p21 | | (SEQ ID NO: 49) |
| | Reverse | 5′-CCCTAGGCTGTGCTCACTTC-3′ |
| | | (SEQ ID NO: 50) |
| Human | Forward | 5′-ACCCCTAGAGGGCAAGTACG-3′ |
| p27 | | (SEQ ID NO: 51) |
| | Reverse | 5′-ATCAGTCTTTGGGTCCACCA-3′ |
| | | (SEQ ID NO: 52) |
| Human | Forward | 5′-ACGCCAAAGACAGAGAAGGA-3′ |
| ANKRD1 | | (SEQ ID NO: 53) |
| | Reverse | 5′-TTCTGCCAGTGTAGCACCAG-3′ |
| | | (SEQ ID NO: 54) |
| Human | Forward | 5′-CTCCCTGTTTTTGGAATGGA-3′ |
| Cyr61 | | (SEQ ID NO: 55) |
| | Reverse | 5′-TGGTCTTGCTGCATTTCTTG-3′ |
| | | (SEQ ID NO: 56) |
| Human- | Forward | 5′-TAGAAACAGCTCGTTGTACCGCT |
| specific | | GGGACCT-3′ |
| β- | | (SEQ ID NO: 57) |
| catenin | Reverse | 5′-GCACTGCCATTTTAGCTCCTTCT |
| | | TGATGTAAT-3′ |
| | | (SEQ ID NO: 58) |
| Human- | Forward | 5′-CCACCAGTCCACCAGTGCAGCAG |
| specific | | AATA-3′ |
| YAP | | (SEQ ID NO: 59) |
| | Reverse | 5′-GCAGTCGCATCTGTTGCTGCTGG |
| | | TTGGAGT-3′ |
| | | (SEQ ID NO: 60) |
| Human- | Forward | 5′-TGGACTCAACAGATCTGTCTGCC |
| specific | | TGCAATC-3′ |
| MET | | (SEQ ID NO: 61) |
| | Reverse | 5′-AATGTACTGTATTGTGTTGTCCC |
| | | GTGGCCA-3′ |
| | | (SEQ ID NO: 62) |
| Human | Forward | 5′-TCCTGCACCACCAACTGCTT-3′ |
| GAPDH | | (SEQ ID NO: 63) |
| | Reverse | 5′-TGGCAGTGATGGCATGGAC-3′ |
| | | (SEQ ID NO: 64) |
| Human | Forward | 5′-CCTGGTGCTCCGTCTTAGAG-3′ |
| UBC | | (SEQ ID NO: 65) |
| | Reverse | 5′-TTTCCCAGCAAAGATCAACC-3′ |
| | | (SEQ ID NO: 66) |
| Human | Forward | 5′-AGAGCTGGTCCAGGCAGTGCAGC |
| Met | | ATGTAGT-3′ |
| | | (SEQ ID NO: 67) |
| | Reverse | 5′-AATCTTTCATGATGATTCCCTCG |
| | | GTCAGAA-3′ |
| | | (SEQ ID NO: 68) |
| Mouse | Forward | 5′-TGATTCGGCTGACAGAAGTG-3′ |
| GREB1 | | (SEQ ID NO: 69) |
| | Reverse | 5′-TGATGGTCTGAGGGTGTGAA-3′ |
| | | (SEQ ID NO: 70) |
| Mouse | Forward | 5′-GCACTGTCCAAGCAAAGCTGCGC |
| AFP | | TCTCTAC-3′ |
| | | (SEQ ID NO: 71) |
| | Reverse | 5′-GCTGATACCAGAGTTCACAGGGC |
| | | TTGCTTC-3′ |
| | | (SEQ ID NO: 72) |
| Mouse | Forward | 5′-GAACCATGTCTGTGCCCAAGGGT |
| GPC3 | | AAAGTTC-3′ |
| | | (SEQ ID NO: 73) |
| | Reverse | 5′-CGCTGTGAGAGGTGGTGATCTCG |
| | | TTGTCCT-3′ |
| | | (SEQ ID NO: 74) |
| Mouse | Forward | 5′-GATCTGGACCCCGGGCAGACTCT |
| TACSTD1 | | GATTTAC-3′ |
| | | (SEQ ID NO: 75) |
| | Reverse | 5′-CAGCACTCAGCACGGCTAGGCAT |
| | | TAAGCTC-3′ |
| | | (SEQ ID NO: 76) |
| Mouse | Forward | 5′-TGGCTGTGTCAATGGAGTCTGCA |
| DLK1 | | AGG-3′ |
| | | (SEQ ID NO: 77) |
| | Reverse | 5′-TGCTGGCAGGGAGAACCATTGAT |
| | | CACG-3′ |
| | | (SEQ ID NO: 78) |
| Mouse | Forward | 5′-ACTCCCTACCTTTTGGTGAGTTG |
| PEG3 | | CTTGCAG-3′ |
| | | (SEQ ID NO: 79) |
| | Reverse | 5′-CTTGGATGAAACGTTCTTGGGCA |
| | | TAACTGG-3′ |
| | | (SEQ ID NO: 80) |
| Mouse | Forward | 5′-AGGCAAGGATAGGCCCAGGAGTA |
| BEX1 | | ATGGAGT-3′ |
| | | (SEQ ID NO: 81) |
| | Reverse | 5′-CTCCCCAACCCTCTGCATCAGGT |
| | | CCCATCT-3′ |
| | | (SEQ ID NO: 82) |
| Mouse | Forward | 5′-TATCTGGACATTGAAGCTTGGAA |
| MEG3 | | AGCCAGT-3′ |
| | | (SEQ ID NO: 83) |
| | Reverse | 5′-TTCATGACCACAGCCCATGGTAT |
| | | CACACAG-3′ |
| | | (SEQ ID NO: 84) |

### 1-14. Formation of Complex and Immunoprecipitation

HepG2 cells (100 mm diameter dish) were lysed with 400 μl of a cell lysis buffer (10 mM Tris-HCl [pH 7.4], 140 mM NaCl, 5 mM EDTA, 1% NP40, 25 mM NaF, 20 mg/ml leupeptin, 20 mg/ml aprotinin, and 10 mM PMSF). The resulting lysate was immunoprecipitated with anti-Smad2/3 antibody, and the immunoprecipitates were probed with predetermined antibodies.

X293T cells (60 mm diameter dish) transfected with HA-FLAG-GREB1, a GFP-GREB1 mutant, a GFP-Smad2 mutant. HA-Smad3, GFP-Smad3, GFP-Smad4, or GFP-Smad7 were lysed with 400 μl of cell lysis buffer, and states of complexes of GREB1, Smad3, Smad4, and Smad7 were analyzed. The resulting lysates were immunoprecipitated with an anti-GFP antibody, and the immunoprecipitates were probed with predetermined antibodies.

In addition, amino acid sequences that form fusion proteins used in this study are as follows: the amino acid sequence of the HA moiety is represented by SEQ ID NO: 85: the amino acid sequence of the FLAG moiety is represented by SEQ ID NO: 86; the amino acid sequence of the GFP moiety is represented by SEQ ID NO: 87; GREB1 mutant (mouse GREB1 Δ310-319 (ΔNLS)) is a GREB1 mutant in which amino acids at positions 310 to 319 of mouse GREB1 are deleted, and the amino acid sequence of the mutant is represented by SEQ ID NO: 88; GREB1 mutant (mouse GREB1 1-666 (N)) is a GREB1 mutant consisting of an amino acid sequence of positions 1 to 666 of mouse GREB1, and the amino acid sequence is represented by SEQ ID NO: 89; GREB1 mutant (mouse GREB1 667-1333 (I)) is a GREB1 mutant consisting of an amino acid sequence of positions 667 to 1333 of mouse GREB1, and the amino acid sequence is represented by SEQ ID NO: 90; GREB1 mutant (mouse GREB1 1334-1954 (C)) is a GREB1 mutant consisting of an amino acid sequence of positions 1334 to 1954 of mouse GREB1, and the amino acid sequence is represented by SEQ 1D NO: 91; GREB1 mutant (mouse GREB1 Δ667-1333 (ΔM)) is a GREB1 mutant in which amino acids at positions 667 to 1333 of mouse GREB1 are deleted, and the amino acid sequence is represented by SEQ ID NO: 92; the amino acid sequence of human Smad2 moiety is represented by SEQ ID NO: 93; human Smad2 mutant (human Smad2 1-265 (N)) is a Smad2 mutant consisting of an amino acid sequence of positions 1 to 265 of human Smad2, and the amino acid sequence is represented by SEQ ID NO: 94; human Smad2 mutant (human Smad2 266-467 (C)) is a Smad2 mutant consisting of an amino acid sequence of positions 266 to 467 of human Smad2, and the amino acid sequence is represented by SEQ ID NO: 95; the amino acid sequence of human Smad3 moiety is represented by SEQ ID NO: 96; the amino acid sequence of human Smad4 moiety is represented by SEQ ID NO: 97 and the amino acid sequence of human Smad7 moiety is represented by SEQ ID NO: 98. In addition, in other studies, each fusion protein used was prepared by selecting a combination of the above-described amino acid sequences and linking the selected sequences to each other.

### 1-15. GST Pull-Down Assay

To analyze the binding between recombinant Smad2/MH1 or recombinant Smad2/MH2 and GREB1, total cell lysates of HepG2 cells or Huh6 cells expressing GFP-GREB1 were incubated with 20 μg of glutathione S-transferase (GST) bound to glutathione-Sepharose beads, GST-Smad2/MH1, or GST-Smad2/MH2 for 1 hour. After sedimentation, the beads were washed three times with a cell lysis buffer (10 mM Tris-HCl [pH 7.4], 140 mM NaCl, 5 mM EDTA, 1% NP40, 25 mM NaF, 20 mg/ml leupeptin, 20 mg/ml aprotinin, and 10 mM PMSF), and the resulting precipitates were probed with an anti-GREB1 antibody or an anti-GFP antibody.

In addition, the amino acid sequences that constitute fusion proteins used in this study are as follows: the amino acid sequence of GST moiety is represented by SEQ ID NO: 99; the amino acid sequence of Smad2/MH1 is represented by SEQ ID NO: 100; and the amino acid sequence of Smad2/MH2 is represented by SEQ ID NO: 101.

### 1-16. Labeling of Cells Using Ethynyl Uridine (EU)

GFP-Smad3-expressing cells with or without expression of HA-FLAG-GREB1 were treated with 1 mM of EU for 30 minutes, followed by fixation, and detection using Click-iT RNA Alexa Fluor 594 Imaging Kit (Thermo Fisher Scientific, Waltham, Mass., USA) was performed.

### 1-17. Analysis of Xenograft Tumor Formation

After 5-week old male BALB/cAnNCrj-nu nude mice (Charles River Laboratory Japan Inc, Osaka, Japan) was anesthetized with medetomidine (0.3 mg/kg body weight) and midazolam (4 mg/kg body weight), HepG2 cells (7×10⁶ cells) suspended in 100 μl of Matrigel high concentration (Corning, N.Y., USA) was injected subcutaneously in the back. Next, 28 days after grafting, the nude mice were sacrificed, areas containing the grafted cells were measured, and subjected to treatment for immunohistochemical analysis. All protocols associated with all animal experiments in this study were carried out after receiving approvals from the Animal Experiment Committee of Osaka University (No. 2867).

### 1-18. Analysis of Xenograft Subcutaneous Tumorigenesis

To 5-week-old BALB/cAJcl-nu/nu mice (nude mice; CLEA Japan), medetomidine (0.3 mg/kg) and midazolam (4 mg/kg) were administered in combination to give anesthesia. Next, Hep3B cells or JHH7 cells (1×10⁷ cells) were suspended in 150 μl of Matrigel high concentration (Corning), and grafted into the mice subcutaneously. From day 3 post grafting, ASO (50 μg/body; about 2.5 mg/kg) was injected subcutaneously at a frequency of twice every one week. The mice grafted with Hep3B cells were euthanized 6.5 weeks after grafting. The mice grafted with JHH7 cells were euthanized 2 weeks after grafting. Tumors were collected from the euthanized mice, and subjected to histological analysis. All protocols associated with all animal experiments in this study were carried out after receiving approvals from the Animal Experiment Committee of Osaka University (No. 26-032-048).

### 1-19. Preparation of Knockout Cells

The target sequence of human GREB1 5′-CTTCTCGGTGTTGAAGCCGA-3′ (SEQ 1D NO: 102) was designed using CRISPR Genome Engineering Resources (http://www.genome-engineering.org/crispr/). By ligating a predetermined oligonucleotide into a BbsI site of pX330 (addgene #42230), a plasmid expressing hCas9 and a single guide RNA (sgRNA) was constructed. Using Lipofectamine LTX reagent (Life Technologies/Thermo Fisher Scientific), the plasmid pX330 containing a sgRNA sequence targeting GREB1 or MITF and having blasticidin resistance was introduced into HepG2 cells, Hep3B cells, JHH7 cells, or COLO679 cells according to protocols recommended by the manufacturer, and culturing the cells in a medium containing 5 μg/mL of blasticidin S for 2 days to select GREB1 knockout cells or MITF knockout cells. Next, a single colony was harvested and mechanically dissociated, and the dissociated cells were seeded again in each well of a 24-well plate.

Further, to produce Smad2 and Smad3 (Smad2/3) double knockout cells, a pRP[CRISPR] plasmid containing hCas9 and dual guide RNAs was designed and synthesized by VectorBuilder Inc. (Guangzhou, China). A sequence of 5′-TATATTGCCGATTATGGCGC-3′ (SEQ ID NO: 103) as a target sequence of gRNA for human Smad2 and a sequence of 5′-GGAATGTCTCCCCGACGCGC-3′ (SEQ ID NO: 104) as a target sequence of gRNA for human Smad3 were designed. The plasmid pRP[CRISPR] containing dual guide RNA sequences targeting Smad2/3 was introduced into HepG2 cells, then a single colony was harvested and mechanically dissociated, and the dissociated cells were seeded again in each well of a 24-well plate.

### 1-20. Chromatin Immunoprecipitation (ChIP) Assay

Confluent HepG2 cells in a 10 cm dish were stimulated in the presence or absence of 5 ng/ml of TGFβ1 for 3 hours. Thereafter, the cells were allowed to crosslink using 1% formaldehyde at room temperature for 10 minutes, and the crosslinking reaction was terminated with 0.125 M glycine. The cells were washed three times with cold PBS, and thereafter the cells were removed and collected. Further, the cells were sedimented by centrifugation, and lysed with a sodium dodecyl sulfate (SDS) lysis buffer (50 mM Tris/HCl [pH 8.0], 10 mM EDTA, and 0.5% SDS). The cell lysates were subjected to ultrasonication to shear DNA into DNA fragments having sizes of 200 to 1000 bp. Supernatant of the sheared chromatin was diluted with ChIP dilution buffer (16.7 mM Tris/HCl [pH 8.0], 167 mM NaCl, 1.2 mM EDTA, and 1.1% Triton X-100) containing a protease inhibitor and a phosphatase inhibitor, and precleared by adding salmon sperm DNA/protein A-agarose beads (Millipore, Billerica, Mass., USA). Next, anti-Tcf-4 antibody, anti-β-catenin antibody, anti-acetylated histone H4 antibody, or negative control IgG (Diagenode, Liege, Belgium) was added, and incubated at 4° C. for 12 hours. Immune complexes adsorbed to the salmon sperm DNA/protein A-agarose beads were washed once with a high-salt buffer (20 mM Tris/HCl [pH 8.0], 500 mM NaCl, 0.1% SDS, 1% Triton X-100, and 2 mM EDTA), further washed once with a LiCl buffer (10 mM Tris/HCl [pH 8.0], 0.25 M LiCl, 1 mM EDTA, 1% deoxycholic acid, and 1% Nonidet P-40), and washed four times with a TE buffer (10 mM Tris/HCl (pH 8.0), and 1 mM EDTA). Next, by incubating in an elution buffer (50 mM Tris/HCl [pH 8.0], 10 mM EDTA, 1% SDS) at 65° C. for 4 hours, the immune complexes were eluted from the beads, and de-crosslinking was performed. Next, RNase A was added to the sample and the sample was treated at 37° C. for 30 minutes, and further proteinase K was added to the sample and the sample was treated at 55° C. for 1 hour. Then, DNA was purified by phenol-chloroform extraction, and PCR was performed using primers shown in Table 3.

TABLE 3

| | | Sequence |
|---|---|---|
| Tc4 | Forward | 5′-TGGATGATACACAGATTGCTACCAAC-3′ |
| binding | | (SEQ ID NO: 105) |
| site of | | |
| Human | Reverse | 5′-GCTTGCCTTTCTCCCTGCACTAAGC-3′ |
| GREB1 | | (SEQ ID NO: 106) |
| Human | Forward | 5′-ACCGCAACGTGGTTTTCTCACCCTATGG- |
| PAI-1 | | 3′ |
| exon 2 | | (SEQ ID NO: 107) |
| | Reverse | 5′-AATCTTGAATCCCATAGCTGCTTGAATC- |
| | | 3′ |
| | | (SEQ ID NO: 108) |

1-21. Construction of Plasmid, and Transfection Using Lentivirus Having cDNA

Using standard recombinant DNA technology, plasmids having full-length GREB1 or various mutants thereof were designed. To produce NSL (nuclear localization sequence)-fused GREB1 mutant, 3 copies of NSL derived from SV40 T antigen were ligated to N terminal of a GFP-GREB1 mutant.

Into CSII-CMV-MCS-IRES2-Bsd provided from Dr. H. Miyoshi (RIKEN BioResource Center, Ibaraki, Japan), GFP and pEGFPC1-GREB1 were subcloned to construct a lentiviral vector.

Next, using FuGENE HD transfection reagent (Roche Applied Science, Basel, Switzerland), the lentiviral vector was transfected into X293T cells together with packaging vector pCAG-HIV-gp and pCMV-VSV-G-RSV-Rev to prepare a lentivirus. Further, 5×10⁴ cells of HepG2 cells were introduced into each well of 12-well plate, treated with the lentivirus and 10 μg/ml of polybrene, centrifuged at 1200×g for 30 minutes, and thereafter incubated for 24 hours to produce HepG2 cells that stably express GFP or GFP-GREB1 (GFP-fused GREB1).

pT3-EF1a-cMET was purchased from Addgene (Cambridge, Mass., USA). pLIVE-SB13 vector was provided by Dr. Toru Okamoto (Osaka University). pT2BH-ΔN90 β-catenin-Luc was produced by in-fusion cloning of CAG promoter, human CTNNB1 sequence that lacks amino acids at positions 1 to 90, and luciferase into pT2BH vector (Addgene). pT2BH-YAPS127A was constructed by inserting a FLAG-tagged human YAPS127A fragment into pT2BH vector between an EcoRI site and a NotI site. pT2BH-GFP2ALuc mouse Greb1 shRNA was constructed by inserting U6 promoter and an mGreb1 shRNA fragment amplified from Mission shRNA (TRCN0000216019) (Sigma-Aldrich) into pT2BH-GFP2ALuc between a PstI site and a Hind III site.

### 1-22. Statistical Analysis

Each study was carried out at least three times. Statistical analysis was performed using Excel (Microsoft, Redmond, Wash., USA) and GraphPad Prism 7 (GraphPad Software, La Jolla, Calif., USA). When P value was less than 0.05, the difference between the results was considered to be statistically significant.

### 2. Results of Study

### 2-1. In Human Hepatoblastoma, GREB1 was Downstream Target Gene of Wnt/β-Catenin Signal

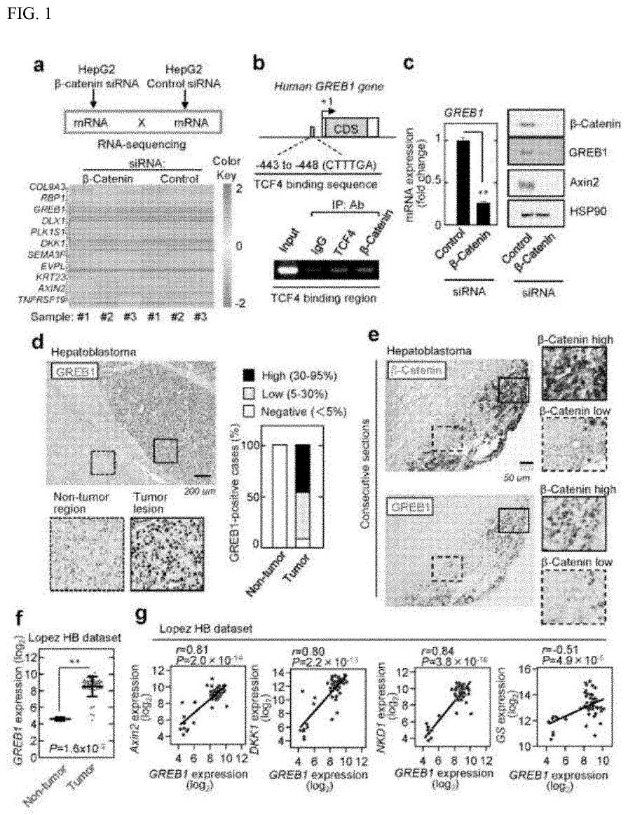

To elucidate the tumorigenesis mechanism of hepatoblastoma, a novel downstream target gene of Wnt/β-catenin signal was screened using HepG2 hepatoblastoma cells in which β-catenin gene had truncated mutations at exons 3 and 4. In HepG2 cells transfected with control siRNA or siRNA against β-catenin (β-catenin siRNA), RNA sequence analysis was performed. As a result, in 8929 genes, 76 genes were selected as candidate genes based on the following criteria: expression level was high (FPKM≥3), and in the cells transfected with β-catenin siRNA (β-catenin knockdown cells), expression level was decreased 3-fold or more as compared to the cells transfected with control siRNA (control cells) (see a of FIG. 1). Further, the candidate genes were narrowed down based on the presence of a DNA binding site of TCF7L2 (TCF4) found by ChIP sequence analysis in HepG2 using a gene set of ENCODE Transcription Factor Binding Site Profiles (TCF7L2#HepG2#hg19#1), and 11 genes were selected (see a of FIG. 1, and Table 4). In the lower image of a of FIG. 1, a heat map of candidate genes in which expression levels of the genes in control cells varied from those in β-catenin knockdown cells are shown. The genes shown in the lower left of a of FIG. 1 represent randomly selected 11 genes among the 76 varied candidate genes.

TABLE 4

| | | EntrezGene | Fold change | |
|---|---|---|---|---|
| Rank | Gene symbol | ID | (KD/control) | P-value |
| 1 | NKD1 | 85407 | −12.814 | .0000114 |
| 2 | LGR5 | 8549 | −9.022 | .0000057 |
| 3 | TNFRSF19 | 55504 | −6.54 | .00000068 |
| 4 | SP5 | 389058 | −5.755 | .000165 |
| 5 | ZNRF3 | 84133 | −4.951 | .000254 |
| 6 | RNF43 | 54894 | −4.191 | .0000245 |
| 7 | AXIN2 | 8313 | −3.702 | .0000362 |
| 8 | GREB1 | 9687 | −3.620 | .000114 |
| 9 | CCND1 | 595 | −3.518 | .0000273 |
| 10 | DKK1 | 22943 | −3.502 | .000273 |
| 11 | LYZ | 4069 | −3.037 | .00000142 |

Most selected candidate genes including NKD1, LGR5, SP5, ZNRF3, RNF43, Axin2, CCND1, and DKK1 were known as target genes of Wnt/β-catenin signal. GREB1, which is one of the candidate genes, is an estrogen-responsive gene in the estrogen receptor regulatory pathway, and it is known that the gene participates in hormone-dependent cancer cell proliferation in breast cancer and prostate cancer. However, it has been previously unknown that whether the gene acts as a downstream target gene of Wnt/β-catenin signal, and participates in tumorigenesis of a cancer that does not express an estrogen receptor. Thus, the present inventors have focused on GREB1 and performed further analysis.

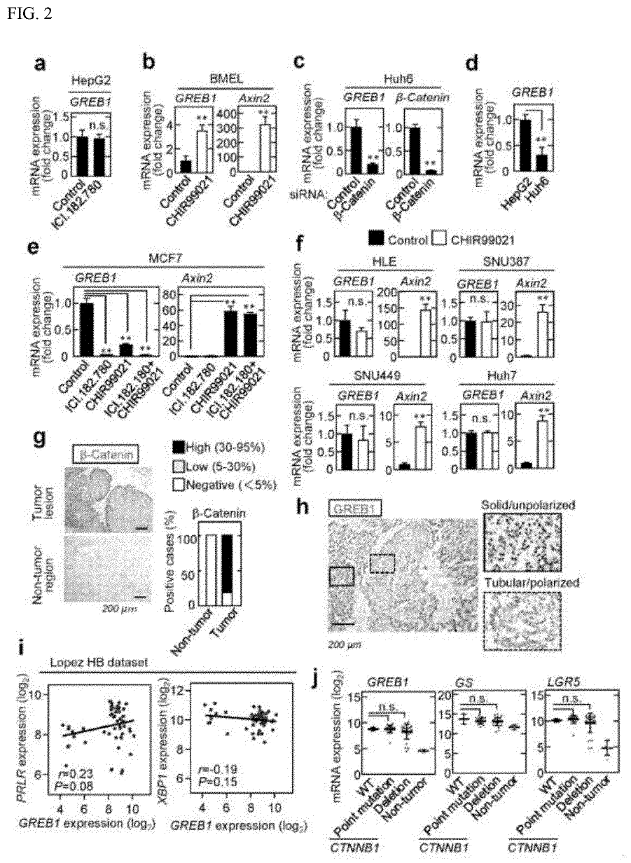

By chromatin immunoprecipitation assay, it is revealed that TCF4 and β-catenin form complexes with a TCF4 binding site present in a 5′-upstream region −443 to −448 of human GREB1 gene (see b of FIG. 1). In fact, in β-catenin knockdown HepG2 cells, a decrease in the GREB1 expression level and a decrease in the amount of the protein were demonstrated (see c of FIG. 1). On the other hand, when HepG2 cells was treated with an estrogen receptor antagonist ICI-182,786 for 48 hours, and thereafter the expression level of GREB1 mRNA was measured by real-time PCR analysis, it was confirmed that the expression level of GREB1 was not affected by ICI-182,786 (see a of FIG. 2). In mouse fetal liver cells (BMEL) cells having dual differentiation potential, the expression of GREB1 was increased as in Axin2 by CHIR99021 treatment having a β-catenin signal activation activity (see b of FIG. 2). In Huh6 cells, which are cells of a hepatoblastoma cell line having a G34V somatic cell mutation in CTNNB1 (β-catenin) gene, the expression level of GREB1 was decreased by knockdown of β-catenin (see c of FIG. 2). In Huh6, the expression level of GREB1 mRNA was low as compared to HepG2 (see d of FIG. 2). In MCF7, which is an estrogen receptor-positive breast cancer cell line, expression of GREB1 was dramatically decreased by ICI-182,786 treatment. However, expression of Axin2 was increased by treatment with CHIR99021 having a Wnt/β-catenin signal activation activity either in the presence or absence of ICI-182,786, whereas expression of GREB1 was not increased (see e of FIG. 2). Further, in HLE, SNU-387 SNU-449, and Huh7, which are human hepatocellular cancer cells, expression of Axin2 was increased by CHIR99021 treatment, whereas expression of GREB1 was not increased (see f of FIG. 2). From these results, it is suggested that GREB1 is a downstream target gene of Wnt/β-catenin signal specific to hepatoblastoma cells or immature liver progenitor cells.

An analysis was performed in 11 cases of hepatoblastoma. Specifically, hepatoblastoma tissues of the 11 cases were stained with anti-GREB1 antibody and hematoxylin. As a result, in 10 cases (90.9%) among the 11 cases, expression of GREB1 was observed in a neoplastic lesion area, whereas expression of GREB1 was not observed in a nonneoplastic area (see Table 5, and d of FIG. 1). Percentages of areas immunostained with the anti-GREB1 antibody with respect to the total area of the neoplastic lesion area were classified into three categories (<5% (Negative), 5 to 30% (Low), and 30 to 95% (High)), and the cases showing ≥5% staining were considered as GREB1-positive. The region specific expression pattern of GREB1 in the hepatoblastoma tissues demonstrated a tendency to show a positive correlation with accumulation of β-catenin in serial sections (see e of FIG. 1). The hepatoblastoma tissues were histologically divided into two types, in which one type of the tissue was formed of solid and unpolarized cells, and the other type was formed of tubular and polarized cells. Among these, GREB1 was highly expressed specifically in the former histological type (solid and unpolarized cells). Further, the hepatoblastoma tissues of the 11 cases were stained with anti-β-catenin antibody and hematoxylin. As a result, it was found that the expression level of β-catenin was increased in the neoplastic lesion area as compared to that in the nonneoplastic area (see g of FIG. 2). Percentages of areas immunostained with the anti-β-catenin antibody with respect to the total area of the neoplastic lesion area were classified into three categories (<5% (Negative), 5 to 30% (Low), and 30 to 95% (High)), and the cases showing ≥5% staining were as β-catenin-positive.

TABLE 5

| Patient | Age | | GREB1 | β-Catenin | YAP | | |
|---|---|---|---|---|---|---|---|
| ID | (year) | Sex | expression | expresion | expression | Pathology | PRETEXT |
| #1 | 1 | male | High | High | High | Combined fetal and embryonal | III |
| #2 | 16 | male | Low | High | High | Embryonal | Recurrence |
| #3 | 3 | male | High | High | High | Combined fetal and embryonal | IV |
| #4 | 0 | male | Negative | Negative | Negative | Fetal | III |
| #5 | 8 | female | Low | High | High | Combined fetal and embryonal | IV |
| #6 | 9 | female | Low | High | High | Embryonal | IV |
| #7 | 0 | male | Low | High | High | Fetal | III |
| #8 | 2 | male | High | High | High | Embryonal | IV |
| #9 | 2 | female | High | High | High | Fetal | III |
| #10 | 4 | male | High | Negative | Negative | Combined fetal and embryonal | III |
| #11 | 2 | female | Low | High | High | Fetal | III |

Further, using a public dataset of hepatoblastoma patients obtained from R2 genomics and a visualization platform database (http://r2.amc.nl), the correlation between the expression of GREB1 gene and the expression of a target gene of Wnt/β-catenin signal in hepatoblastoma was analyzed. Two parameters associated with the GREB1 gene expression and the target gene expression were available with respect to 50 neoplastic lesion areas and 5 nonneoplastic areas in hepatoblastoma cases. As a result of the analysis, it was found that, in the neoplastic lesion area, GREB1 mRNA was significantly upregulated as compared to that in the region of the nonneoplastic area (see f of FIG. 1). Further, a significant positive correlation was demonstrated between the expression levels of the Wnt/β-catenin signal target genes, such as Axin2, DKK1, NKD1, and glutamine synthetase (GS), and the expression level of GREB1 (see g of FIG. 1). On the other hand, expression of downstream target genes of estrogen receptors, such as PRLR and XBP1, was not changed significantly (see i of FIG. 2). That is, from these results, it is confirmed that, in hepatoblastoma, activation of Wnt/β-catenin signal, rather than estrogen receptor signal, causes increase in expression of GREB1.

According to a public dataset of hepatoblastoma patients, in cases in which the region of exon 3 or exon 4 of CTNNB1 gene in hepatoblastoma had a mutation or a deletion, expression of GREB1 was not significantly changed as compared to that in cases without mutation (see j of FIG. 2). Similarly, in Axin2 and GS, which are downstream target genes of β-catenin, the expressions were not changed regardless of whether an abnormality of CTNNB1 gene was present or not. From these results, it is revealed that the expression of GREB1 in hepatoblastoma is correlated with the activity of β-catenin signal, but does not necessarily associate with a mutation in the region of exon 3 or exon 4 of CTNNB1 gene. Thus, it is suggested that a different types of β-catenin signal activation independent of CTNNB1 gene mutations or CTNNB1 gene mutations may occur.

### 2-2. GREB1 Expression Participates in Hepatoblastoma Cell Proliferation

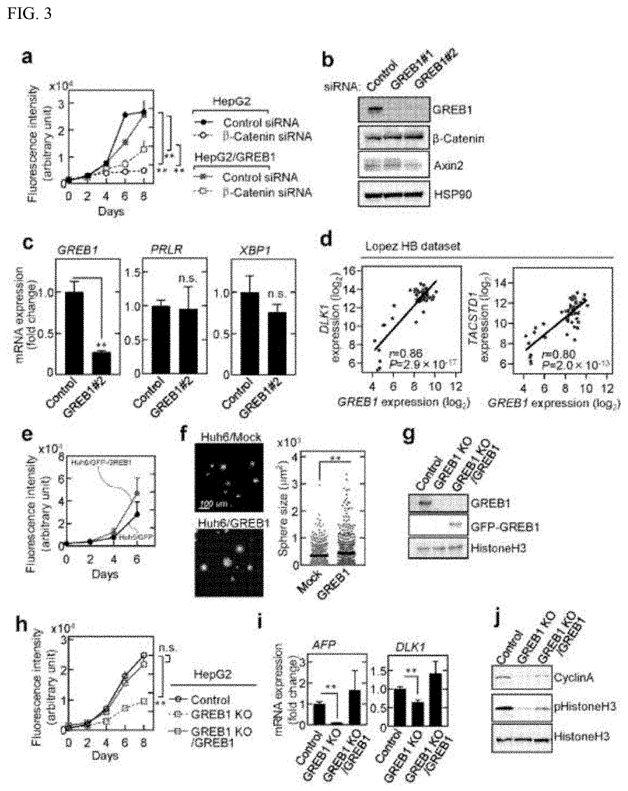

As reported previously, knockdown of β-catenin decreased HepG2 cell proliferation (see a of FIG. 3). Further, expression of exogenous GREB1 partially restored the phenotype by the knockdown of β-catenin (see a of FIG. 3). Thus, to elucidate the role of GREB1 in HepG2 cells, knockdown of GREB1 was carried out using two different siRNAs. As a result of probing lysates of GREB1 knockdown cells with anti-GREB1 antibody, anti-Axin2 antibody, anti-β-catenin antibody, and anti-HSP90 antibody, it was demonstrated that although siRNAs (GREB1 #1 siRNA and GREB1 #2 siRNA) knocked down GREB1 (see b of FIG. 3), the knockdown of GREB1 had no effect on the expressions of PRLR and XBP1 as ER signal target genes (see c of FIG. 3) and also had no effect on the expressions of β-catenin and Axin2 (see b of FIG. 3). That is, these results suggest that GREB1 is not a gene that functions upstream of ER and Wnt/β-catenin signals.

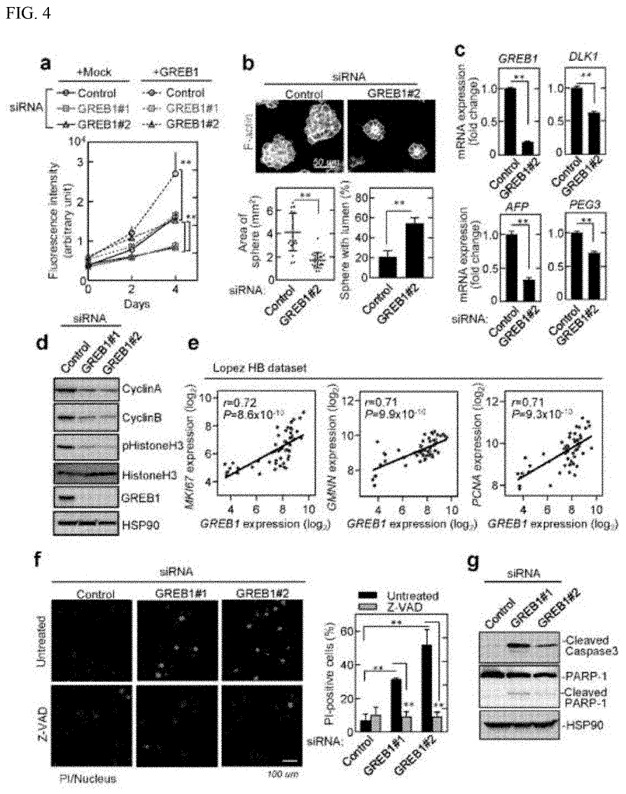

Mock HepG2 cells (having a vector only, containing no GREB1) or GREB1-transduced HepG2 cells were transfected with control siRNA or two different types of siRNA (GREB1 #1 siRNA and GREB1 #2 siRNA), then two-dimensional culture (plastic dish culture) was performed, and a relative cell number was calculated by Cyquant assay overtime. As a result, in GREB1 knockdown, proliferative capacity of HepG2 cells in two-dimensional culture was reduced. However, when GREB1-transduced HepG2 cells were transfected with siRNAs against GREB1 (GREB1 #1 siRNA/GREB1 and GREB1 #2 siRNA/GREB1), the proliferative capacity of HepG2 cells was not affected (see a of FIG. 4).

Further, HepG2 cells transfected with control siRNA or siRNA (GREB1 #2 siRNA) were cultured in 3D Matrigel for 5 days, and the cultured cells were stained with phalloidin to measure the areas of spheres (n=20). As a result, knockdown of GREB1 reduced the area of sphere to half (b of FIG. 4). On the other hand, in the case of GREB1 knockdown, it was found that the ratio of spheres having polarized lumens was increased (b of FIG. 4). The ratio of spheres having polarized lumens was calculated as a ratio of spheres having F-actin-positive central microlumen to the total spheres. From these results, it was suggested that GREB1 knockdown HepG2 cells were transformed into a differentiation state having epithelial polarization. These results were in agreement with the result that GREB1 was specifically expressed in solid unpolarized cells in hepatoblastoma tissues (see h of FIG. 2). In fact, GREB1 knockdown significantly decreased the expressions of DLK1, AFP, and PEG3, which are undifferentiated hepatic progenitor cell marker genes (c of FIG. 4). In agreement with these results, also in a public dataset of hepatoblastoma patients, a significant positive correlation is confirmed between the expression levels of hepatoblastoma marker genes, such as DLK1 and TACSTD1, and the expression level of GREB1 (see d of FIG. 3).

Further, in Huh6 cells having a low GREB1 expression level transfected with GFP or GFP-GREB1, when GFP-GREB1 was introduced and GREB1 was overexpressed, increases in proliferative capacity were observed in two-dimensional culture and three-dimensional culture (see e and f of FIG. 3). That is, from these results, it is revealed that expression of GREB1 in hepatoblastoma cells constitutes one factor in increase in the proliferative capacity.

HepG2 cells transfected with control siRNA or GREB1 siRNAs (GREB1 #1 siRNA and GREB1 #2 siRNA) were cultured in a medium containing 1% FBS for 1 day, and the cell lysates were probed with anti-cyclinA antibody, anti-cyclin B antibody, anti-phosphorylated histone H3 antibody, anti-histone H3 antibody, anti-GREB1 antibody, and anti-HSP90 antibody. As a result, it is revealed that knockdown of GREB1 decreases expression of cell cycle regulators including cyclinA, cyclinB, and phosphorylated histone H3 (see d of FIG. 4).

Further, by analysis using a public database, in hepatoblastoma cells, it is found that there is a positive correlation between the expression level of GREB1 and the expression levels of MKI67, GMMN, and PCNA (see e of FIG. 4).

Further, the phenotypes in expression of hepatoblastoma markers, cell proliferation, and cell cycle in HepG2 cells by knockdown of GREB1 were also demonstrated in GREB1 knockout HepG2 cells produced using CRISPR/Cas9, and the phenotypes were restored by expression of exogenous GREB1 (see g to j of FIG. 3).

Further, HepG2 cells were transfected with control siRNA or siRNAs (GREB1 #1 siRNA and GREB1 #2 siRNA), cultured in media (caspase inhibitor Z-VAD was contained in one condition, and not contained in the other condition) containing 0.1% FBS for 2 days, stained with propidium iodide (PI, living cells) and Hoechst33342 (nucleus), and the cell viability was evaluated. As a result, when a medium which did not contain Z-VAD was used, it was demonstrated that knockdown of GREB1 resulted in elevated cell death of HepG2 cells (see f of FIG. 4).

Further, HepG2 cells were transfected with control siRNA or siRNAs (GREB1 #1 siRNA and GREB1 #2 siRNA), cultured in a medium containing 0.1% FBS for 2 days, and the cell lysates were probed with anti-cleaved caspase 3 antibody, anti-PARP1 antibody, and anti-HSP90 antibody. As a result, it was demonstrated that the amounts of anti-cleaved caspase 3, which is a marker of apoptotic cells, and PARP1 in the cells were increased in GREB1 knockout HepG2 cells (see g of FIG. 4).

That is, from these results, it is suggested that, in hepatoblastoma cells, GREB1 is essential not only for cell proliferation via cell cycle regulation, but also for cell survival.

2-3. GREB1 Forms Complex with Smad2/3

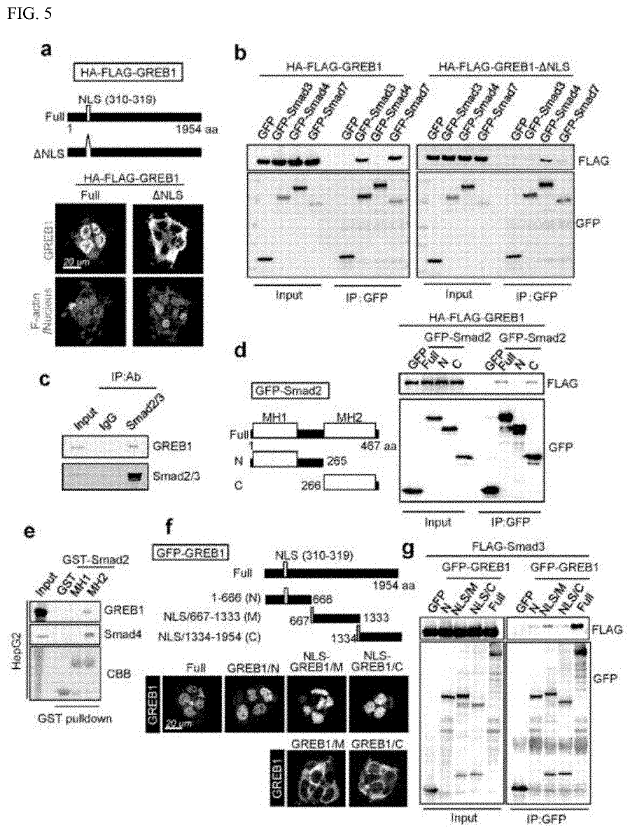

In agreement with a previous report in breast cancer cells, in X293T cells, HA-FLAG-GREB1 mainly localized in the nucleus (see a of FIG. 5). A mutant GREB1 (HA-FLAG-ΔNLS-GREB1) in which amino acids at positions 310 to 319, which is a nuclear localization signal (NLS) of GREB1, were deleted localized in the cytoplasm (see a of FIG. 5). To elucidate the mechanism by which GREB1 regulates the proliferation of hepatoblastoma cells independent of ER signal, candidate proteins that may interact with GREB1 were identified using Biological General Repository for Interaction Datasets (BioGRID) (https://thebiogrid.org/). As a result, as candidate proteins that may interact with GREB1, proteins shown in Table 6 were found.

TABLE 6

| | Interactor | | Dataset |
|---|---|---|---|
| | (Bait) | Experiment | (PMID) |
| 1 | BCAR3 | Two-hybrid | 25640309 |
| 2 | CASP8 | Two-hybrid | 25640309 |
| 3 | CDKN2C | Two-hybrid | 25640309 |
| 4 | DNAJC7 | Affinity capture-MS | 285114442 |
| 5 | DPPA3 | Affinity capture-MS | Pre-publication |
| 6 | HMMR | Two-hybrid | 25640309 |
| 7 | HRAS | Two-hybrid | 25640309 |
| 8 | LSP1 | Two-hybrid | 25640309 |
| 9 | PALB2 | Two-hybrid | 25640309 |
| 10 | RBCC1 | Two-hybrid | 25640309 |
| 11 | SMAD4 | Two-hybrid | 25640309 |
| 12 | TGFβ1 | Two-hybrid | 25640309 |
| 13 | TKT | Affinity capture-MS | 28514442 |
| 14 | TTC9C | Affinity capture-MS | Pre-publication |
| 15 | XRCC3 | Two-hybrid | 25640309 |

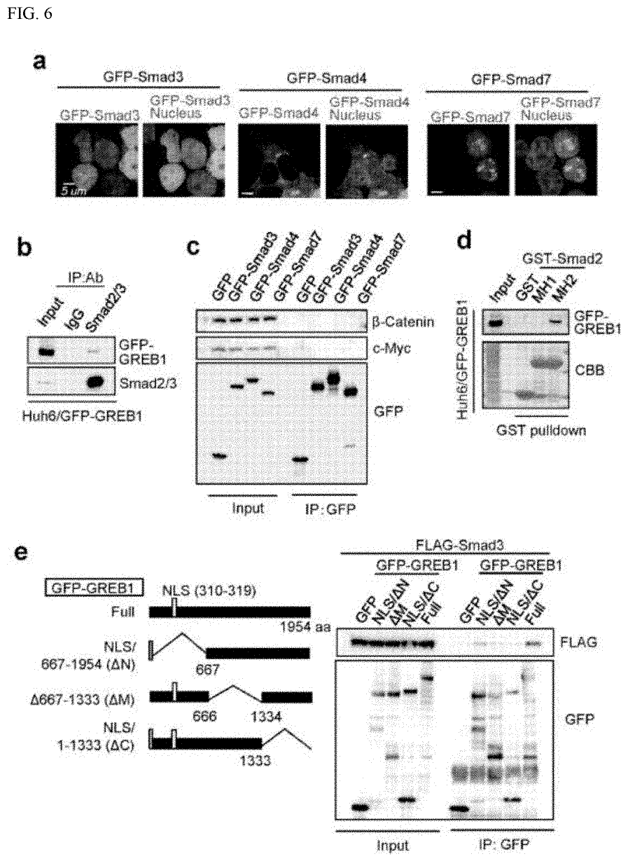

Among these candidate proteins, the present inventors focused on Smad4, which is a central mediator of TGFβ signal. Smad proteins include three classes, depending on the functions, consisting of Smad3 (Co-mediator Smad, Co-Smad), Smad4 (receptor-regulated Smad, R-Smad), and Smad7 (inhibitory Smad, I-Smad). In X293T cells, GFP-Smad3 and GFP-Smad7 were mainly localized in the nucleus, and GFP-Smad4 was localized in the cytoplasm (see a of FIG. 6). Thus, complex formation ability of Smad3, Smad4, and Smad7 with GREB1 was evaluated. Specifically, first, cell lysates of X293T cells expressing HA-FLAG-GREB1, and GFP, GFP-Smad3, GFP-Smad4, or GFP-Smad7 were immunoprecipitated with anti-GFP antibody. Next, the cell lysates (Input) and the immunoprecipitates (IP) were reacted with anti-HA antibody or anti-GFP antibody. As a result, it was demonstrated that HA-FLAG-GREB1 formed complexes with Smad3 (Co-Smad) and Smad7 (I-Smad), whereas did not form complexes with Smad4 (R-Smad)(see b of FIG. 5). On the other hand, it was demonstrated that HA-FLAG-ΔNLS-GREB1 formed complexes with Smad4 (R-Smad), whereas did not form complexes with Smad3 (Co-Smad) and Smad7 (I-Smad) (see b of FIG. 5). From these results, it is suggested that GREB1 binds to all Smad families depending on their intracellular localization. Further, when lysates of HepG2 cells were immunoprecipitated with anti-Smad2/3 antibody and the resulting immunoprecipitates (IP) were reacted with anti-Smad2/3 antibody and anti-GREB1 antibody, biding of GREB1 to endogenous Smad2/3 was demonstrated (see c of FIG. 5), whereas interaction between GREB1, and Smad4 or Smad7 was hardly observed (data not shown). Also, in Huh6 allowed to express GFP-GREB1, when cell lysates were immunoprecipitated with anti-GFP antibody and the resulting immunoprecipitates (IP) were reacted with anti-Smad2/3 antibody and anti-GREB1 antibody, binding of GREB1 to endogenous Smad2/3 was demonstrated (see b of FIG. 6). On the other hand, interaction of β-catenin and c-Myc, which are other nucleus proteins, with GFP-Smad3, GFP-Smad4, or GFP-Smad7 was not observed (see c of FIG. 6). Thus, functional interaction between GREB1 and Smad2/3 was further investigated.

It is known that Smad2 has two functional domain, that is, an MH1 domain and an MH2 domain (seed of FIG. 5). Thus, binding properties of Smad2 with GREB1 were evaluated using full-length Smad2 (Full), a Smad2 mutant (N) consisting only of a N-terminal region containing the MH1 domain in which a C-terminal region (amino acids at positions 266 to 467) was deleted, and a Smad2 mutant (C) consisting only of a C-terminal region containing the MH2 domain in which an N-terminal region (amino acids at positions (1 to 265) was deleted (see d of FIG. 5). First, lysates of X293T cells expressing HA-FLAG-mGREB1, and GFP or GFP-Smad2 (Full, mutant N, or mutant C) were immunoprecipitated with anti-GFP antibody. Next, the cell lysates (input) and the immunoprecipitates (IP) were reacted with anti-HA antibody or anti-GFP antibody. As a result, GFP-Smad2 (mutant C) formed complexes with GREB1, whereas GFP-Smad2 (mutant N) did not form complexes with GREB1 (see d of FIG. 5). In addition, the recombinant GST-Smad2/MH2 domain bound to endogenous GREB1 and GFP-GREB1 expressed in Huh6 cells in a pull-down assay under conditions in which endogenous Smad4 of HepG2 cells bound (see e of FIG. 5). On the other hand, under the same conditions, the recombinant GST-Smad2/MH1 domain did not bind (see e of FIG. 5). Also, in Huh6 cells allowed to express GFP-GREB1, the same tendency was demonstrated (see d of FIG. 6). From these results, it is suggested that GREB1 directly binds to Smad2/3 in hepatoblastoma cells.

Further, to identify the regions where GREB1 interacts with Smad2/3, mutant N (1 to 666) in which amino acids at positions 667 to 1954 of GREB1 were deleted, mutant M (667 to 1333) in which amino acids at positions 1 to 666 and positions 1334 to 1954 of GREB1 were deleted, and mutant C (1334 to 1954) in which amino acids at positions 1 to 1333 of GREB1 were deleted were prepared, and, in addition, NLS-GREB1 mutants (NLS/667-1333 and NLS/1334-1954) in which three copies of NLS sequence (SEQ ID NO: 109) derived from SV40T antigen were bound to the N terminal of the mutants M and C that have no NLS were prepared (see f of FIG. 5). Then, X293T cells expressing the GFP-GREB1 mutants were stained with anti-GFP antibody and Hoechst33342 (see f of FIG. 5). As a result, it was demonstrated that the GREB1 mutants (1 to 666, NLS/667-1333, and NLS/1334-1954) were localized in the nucleus.

Further, lysates of X293T cells expressing FLAG-Smad3, and GFP, GFP-GREB1, or GFP-GREB1 mutant were immunoprecipitated with anti-GFP antibody. Next, the resulting cell lysates (Input) and immunoprecipitates (IP) were reacted with anti-FLAG antibody or anti-GFP antibody. As a result, it was found that, in X293T cells, GREB1 mutant (NLS/667-1333) bound to Smad3, whereas GREB1 mutants (1 to 666 and NLS/1334-1954) hardly bound to Smad3 (see g of FIG. 5).

Further, mutant (NLS/667-1954 (ΔN)) in which amino acids at positions 1 to 666 of GREB1 were deleted and three copies of NLS sequence (SEQ ID NO: 109) derived from SV40T antigen were bound to the N terminal, mutant (Δ667-1333/ΔM) in which amino acids at positions 667 to 1333 of GREB1 were deleted, and mutant (NLS/1-1333 (ΔC) in which amino acids at positions 667 to 1333 of GREB1 were deleted and three copies of NLS sequence (SEQ ID NO: 109) derived from SV40T antigen were bound to the N terminal were prepared. Lysates of X293T cells expressing these mutants were immunoprecipitated with anti-GFP antibody. Next, the resulting cell lysates (input) and immunoprecipitates (IP) were further reacted with anti-FLAG antibody or anti-GFP antibody (see e of FIG. 6). As a result, it was demonstrated that the wild type GREB1 bound to Smad3, whereas the mutants in which amino acids at positions 667 to 1333 of GREB1 were deleted did not bind to Smad3 (see e of FIG. 6). From the above-described results, it is revealed that the amino acid region at positions 667 to 1333 of GREB1 plays an important role in the binding to Smad3. Further, these results suggest that the amino acid region containing amino acids at positions 667 to 1333 of GREB1 interacts with the NH2 domain of Smad2/3 and forms complexes in the nucleus of hepatoblastoma cells.

### 2-4. GREB1 Functions as Negative Regulator of TGFβ Signal

To elucidate the role of GREB1 in TGFβ signal, expressions of target genes, nuclear transport of Smad2, complex formation of Smad2/3 and Smad4, and phosphorylation of endogenous Smad2/3 were investigated.

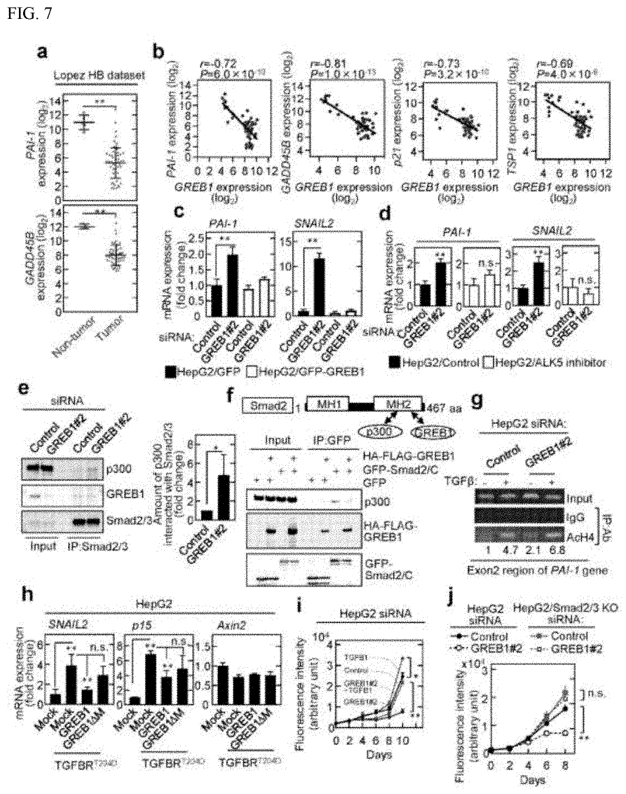

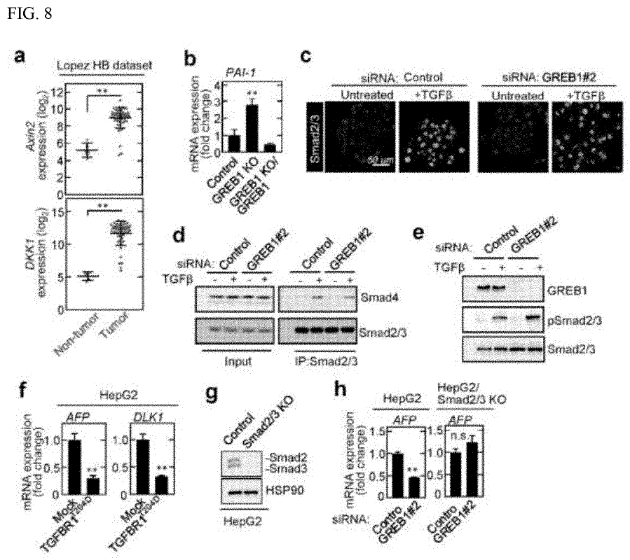

A public dataset of hepatoblastoma patients was analyzed, and the results showed that expression levels of Axin2 and DKK1, which are target genes of β-catenin signal, were significantly increased in tumor lesion sites as compared to those in nonneoplastic areas (see a of FIG. 8). On the other hand, expression levels of PAI-1 or GADD45B, which are target genes of TGFβ signal, were significantly decreased in tumor lesion sites as compared to those in nonneoplastic areas (see a of FIG. 7). Further, it was found that there were significant inverse correlations between the expression level of GREB1 and expression levels of target genes of TGFβ signal (PAI-1, p21/CDKN1A, TSP-1, and CTGF) (see b of FIG. 7). HepG2 cells expressing GFP (HepG2/GFP) or HepG2 cells expressing GFP-GREB1 (HepG2/GFP-GREB1) were transfected with control siRNA or GREB1 #2 siRNA. In these cells, mRNA levels of PAI-1 and SNAIL2 were measured by real-time PCR (n=3). As a result, in GREB1 knockdown HepG2 cells (HepG2/GFP), expression levels of PAI-1 and SNAIL2, which are target genes of TGFβ signal, were increased, and these phenotypes were restored by expression of GFP-GREB1 (see c of FIG. 7).

Further, PAI-1 mRNA levels in HepG2 cells (Control), GREB1 knockout HepG2 cells (GREB1 KO), or GREB1 KO HepG2 cells allowed to express GREB1 (GREB1 KO/GREB1) were analyzed by real-time PCR. As a result, it was found that PAI-1 mRNA levels were significantly increased by knockout of GREB1, and the phenotype by knockout of GREB1 was restored by expression of exogenous GREB1 (see b of FIG. 8).

Further, HepG2 cells were transfected with control siRNA or GREB1 #2 siRNA, cultured in the presence or absence of a TGFβ receptor inhibitor (ALK5 inhibitor), and mRNA levels of PAI-1 and SNAIL2 in the cells were measured. As a result, it was found that the increases in mRNA levels of PAI-1 and SNAIL2 by knockdown of GREB1 were TGFβ receptor signal dependent (see d of FIG. 7).

Further, HepG2 cells transfected with control siRNA or GREB1 #2 siRNA were cultured in the presence or absence of 10 ng/mL of TGFβ for 30 minutes. The cultured cells were fixed, and immunostained with anti-Smad2/3 antibody (see c of FIG. 8). Further, cell lysates of the cultured cells were immunoprecipitated with anti-Smad2/3 antibody. Next, the resulting cell lysates (Input) and immunoprecipitates (IP) were reacted with anti-Smad4 antibody or anti-Smad2/3 antibody (see d of FIG. 8). In addition, the cell lysates of the cultured cells were reacted with anti-GREB1 antibody, anti-phosphorylated Smad2/3 (pSmad2/3) antibody, or anti-Smad2/3 antibody (see e of FIG. 8). As a result, it was found that knockdown of GREB1 had no effect on TGFβ-dependent nuclear transport of Smad2/3, complex formation of Smad2/3 and Smad4, and phosphorylation of Smad2/3 (see c to e of FIG. 8). From these results, it is revealed that GREB1 has an effect of suppressing the function of Smad2/3 in the nucleus, and thereby the TGFβ-dependent gene expression is suppressed.

It is known that transcriptional activators such as CBP and p300 have a histone acetyltransferase (HAT) activity to change chromatin structure. Further, it is also known that R-Smads (Smad2/3) directly interact with CBP or p300 by the MH2 domain. Thus, the effect of knockdown of GREB1 on the binding between a transcriptional activator and Smad2/3 was investigated as follows.

Lysates of HepG2 cells transfected with control siRNA or GREB1 #2 siRNA were immunoprecipitated with anti-Smad2/3 antibody. Next, the resulting cell lysates (input) and immunoprecipitates (IP) were reacted with anti-p300 antibody, anti-GREB1 antibody, or anti-Smad2/3 antibody (see e of FIG. 7). As a result, in HepG2 cells, when GREB1 was knocked down, the binding of Smad2/3 to p300 was increased (see e of FIG. 7).

Further, cell lysates of X293T cells expressing HA-FLAG-GREB1, GFP-Smad2 mutant (C), or GFP were immunoprecipitated with anti-GFP antibody. Next, the resulting cell lysates (Input) and immunoprecipitates (IP) were reacted with anti-p300 antibody, anti-HA antibody, or anti-GFP antibody. As a result, it was demonstrated that, in X293T cells, Smad2/C mutant (C) having an MH2 domain interacted with p300, and overexpression of HA-FLAG-GREB1 inhibited the interaction between Smad2/C mutant (C) and p300 (see f of FIG. 7).

Further, HepG2 cells transfected with control siRNA or GREB1 #2 siRNA were cultured in the presence or absence of 10 ng/mL of TGFβ for 30 minutes. Cell lysates of the cultured cells were immunoprecipitated with anti-acetylated histone 4 (AcH4) antibody. Next, the exon 2 domain of PAI contained in the cell lysates (Input) and immunoprecipitates (IP) was analyzed by PCR using domain-specific primers. As a result, it was demonstrated that knockdown of GREB1 increased the amount of acetylated histone 4 in the PAI-1 gene locus (exon 2) of HepG2 cells (see g of FIG. 7).

In HepG2 cells, when constitutively active TGFBR1 mutant (T204D) was overexpressed together with GFP, GFP-GREB1, or GFP-GREB1 mutant (Δ667-1333/ΔM) and the mRNA expression of SNAIL2 or p15 gene were quantitated, the constitutively active TGFBR1-dependent gene expression of SNAIL2 or p15 was suppressed by expression of GREB1, whereas expression of SNAIL2 or p15 gene was not significantly suppressed by expression of GREB1 mutant (Δ667-1333/ΔM) (see h of FIG. 7). Further, under the same conditions, overexpression of GREB1 had no effect on expression of Axin2 (see h of FIG. 7).

Further, AFP mRNA and DLK1 mRNA levels in HepG2 cells transfected with TGFBR1/T204D mutant (a mutant in which threonine at position 204 of TGFBR1 is substituted by aspartic acid) were analyzed by real-time PCR. As a result, it was found that the expressions of AFP and DLK1 in HepG2 cells were decreased by overexpression of constitutively active TGFBR1 mutant (T204D)(see f of FIG. 8). This result is in agreement with a previous report that TGFβ signal decreases the expressions of AFP and DLK1 in rat fetal liver cells or liver cancer cells.

Further, lysates of HepG2 cells and Smad2/3 knockout HepG2 cells (HepG2/Smad2/3 KO) were probed with anti-Smad2/3 antibody or anti-HSP90 antibody. In addition, the HepG2 cells and the Smad2/3 knockout HepG2 cells (HepG2/Smad2/3 KO) were transfected with control siRNA or siRNA against GREB1 (GREB1 #2 siRNA), and AFP mRNA levels were analyzed by real-time PCR. As a result, knockout of Smad2/3 restored the decrease in expression of AFP by knockdown of GREB1, whereas had no effect on the decrease in expression of DLK1 (see g and h of FIG. 8).

From the above-described results, it is suggested that GREB1 prevents binding of Smad2/3 to p300, and inhibits the expressions of target genes of TGFβ-Smad signal.

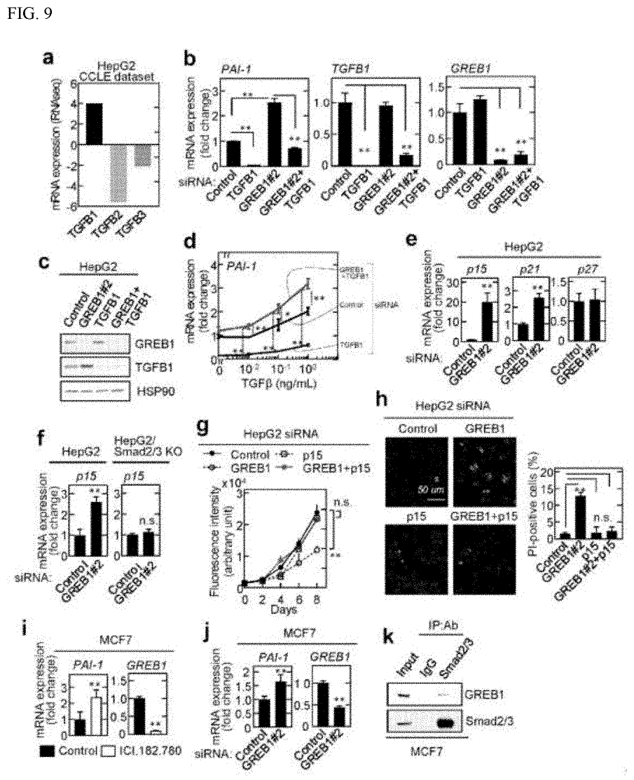

Further, when gene expression levels of TGFβ1, TGFB2, or TGFB3 were analyzed using RNA sequence data of HepG2 in the mRNA profile dataset of Cancer Cell Line Encyclopedia (CCLE) (https://portals.broadinstitute.org/ccle), TGFβ1 was highly expressed in HepG2 cells (see a of FIG. 9).

Further, PAI-1 mRNA level, TGFβ1 mRNA level, and GREB1 mRNA level in HepG2 cells transfected with control siRNA, TGFβ1 siRNA, GREB1 siRNA (GREB1 #2 siRNA), or TGFβ1 siRNA and GREB1 siRNA (GREB1 #2 siRNA) in combination were analyzed by real-time PCR, and the results showed that double knockdown of TGFβ1 and GREB1 significantly suppressed the increase in PAI-1 gene expression by knockdown of GREB1 (see b of FIG. 9).

Further, lysates of HepG2 cells transfected with control siRNA, TGFβ1 siRNA, GREB1 siRNA (GREB1 #2 siRNA), or siRNAs of TGFβ1 and GREB1 (GREB1 #2 siRNA) in combination were probed with anti-GREB1 antibody, anti-TGFB1 antibody, or anti-HSP90 antibody. As a result, it was demonstrated that double knockdown of TGFβ1 and GREB1 efficiently reduced the protein expressions of TGFβ1 and GREB1 (see c of FIG. 9).

Further, two-dimensional culture (plastic dish culture) of HepG2 cells transfected with control siRNA, TGFβ1 siRNA, GREB1 siRNA (GREB1 #2 siRNA), or TGFβ1 siRNA and GREB1 siRNA (GREB1 #2 siRNA) in combination was performed, and the cell number was measured over time. As a result, it was found that the prevention of cell proliferation by knockdown of GREB1 was restored by double knockdown together with TGFβ1 (see i of FIG. 7).

HepG2 cells transfected with control siRNA, TGFβ1 siRNA, GREB1 siRNA (GREB1 #2 siRNA), or TGFβ1 siRNA and GREB1 siRNA (GREB1 #2 siRNA) in combination were cultured in the absence of TGFβ1 at a concentration of 0.01 to 1 ng/ml for 4 hours, and thereafter PAI-1 mRNA levels were analyzed by real-time PCR. As a result, it was demonstrated that TGFβ-dependent PAI-1 mRNA expression was facilitated by knockdown of GREβ1 and TGFβ1 (see d of FIG. 9).

Further, when GREB1 was knocked down in HepG2 cells, among p15, p21, and p27, which are TGFβ signal downstream target genes that regulate suppression of cell proliferation, expression of p15 was dramatically increased. The increase in expression of p15 by knockdown of GREB1 was reduced by knockout of Smad2/3 (see e of FIG. 9). In addition, when p15 was knocked down in HepG2 cells, the phenotypes of suppression of cell proliferation and elevated cell death by the suppression of GREB1 expression were restored (see f of FIG. 9). From these results, it is suggested that p15 is important in the phenotypes of suppression of cell proliferation and elevated cell death by suppression of GREB1 expression. In agreement with these results, knockout of Smad2/3 in HepG2 cells inhibited the suppression of cell proliferation by GREB1 knockdown (see j of FIG. 7).

In addition, two-dimensional culture (plastic dish culture) of HepG2 cells transfected with control siRNA, GREB1 siRNA (GREB1 #2 siRNA), p15 siRNA, or siRNA of GREB1 (GREB1 #2 siRNA) and siRNA of p15 in combination was performed, and the cell number was measured over time. As a result, since the suppression of cell proliferation by knockdown of GREB1 was restored by knockdown of p15, it is revealed that the phenotype is p15 dependent (see g of FIG. 9).

Further, HepG2 cells transfected with control siRNA, GREB1 siRNA (GREB1 #2 siRNA), p15 siRNA, or siRNA of GREB1 (GREB1 #2 siRNA) and siRNA of p15 in combination were cultured in media (caspase inhibitor Z-VAD was contained in one condition, and not contained in the other condition) containing 0.1% FBS for 2 days, stained with propidium iodide (PI) and Hoechst33342, and the cell viability was calculated. As a result, since the elevated cell death by knockdown of GREB1 was restored by knockdown of p15, it is revealed that the phenotype is p15 dependent (see h of FIG. 9).

From these results, it is suggested that GREB1 knockdown increases sensitivity to TGFβ, and suppresses cell proliferation and induces cell death.

Further, in breast cancer cells MCF7, treatment with estrogen receptor antagonist (ICI.182.780) or knockdown of GREB1 suppresses the mRNA expression of PAI-1 (see i and j of FIG. 9). In addition, in MCF7 cells, GREB1 bound to endogenous Smad2/3 (see k of FIG. 9). From these results, it is suggested that GREB1 functions as a negative regulator of TGFβ signal not only in hepatoblastoma cells, but also in breast cancer cells.

### 2-5. Interaction Between GREB1 and Smad2/3 Inhibits Transcription in a Limited Area of Nucleus

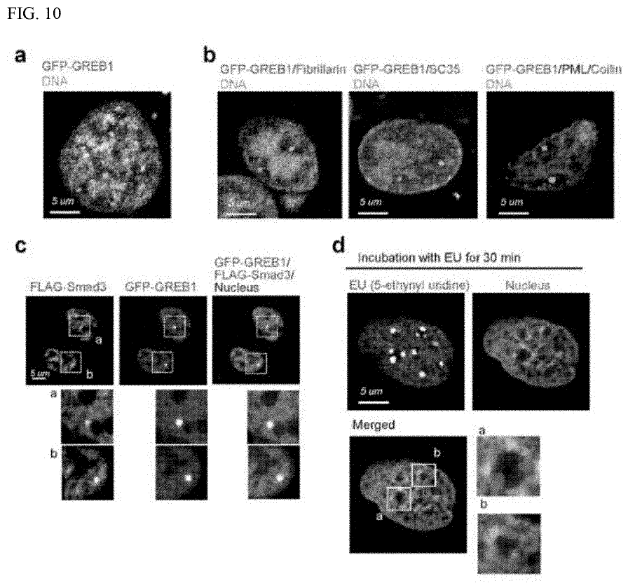

When HepG2 cells expressing GFP-GREB1 were fixed and stained with anti-GFP antibody and Hoechst33342, GFP-GREB1 was observed as dots in a spaces between chromatins, rather than in a chromatin region (see a of FIG. 10). Further, when HepG2 cells expressing GFP-GREB1 were fixed and stained with anti-GFP antibody, anti-Fibrillarin antibody, anti-SC35 antibody, anti-PML antibody, or anti-Coilin antibody, and Hoechst33342, it was confirmed that dots stained with GREB1 did not colocalize with fibrillarin, SC35, PML, and Coilin (see b of FIG. 10). From these results, it is suggested that GREB1 exists independent of nucleolus, nuclear speckle, PML (Promyelocytic leukemia) body, and Cajal body. Further, when HepG2 cells expressing GFP-GREB1 and FLAG-SMAD3 were fixed and stained with anti-GFP antibody, anti-FLAG antibody, and Hoechst33342, it was observed that GFP-GREB1 and FLAG-Smad3 formed complexes and existed in spaces between chromatins (see c of FIG. 10).

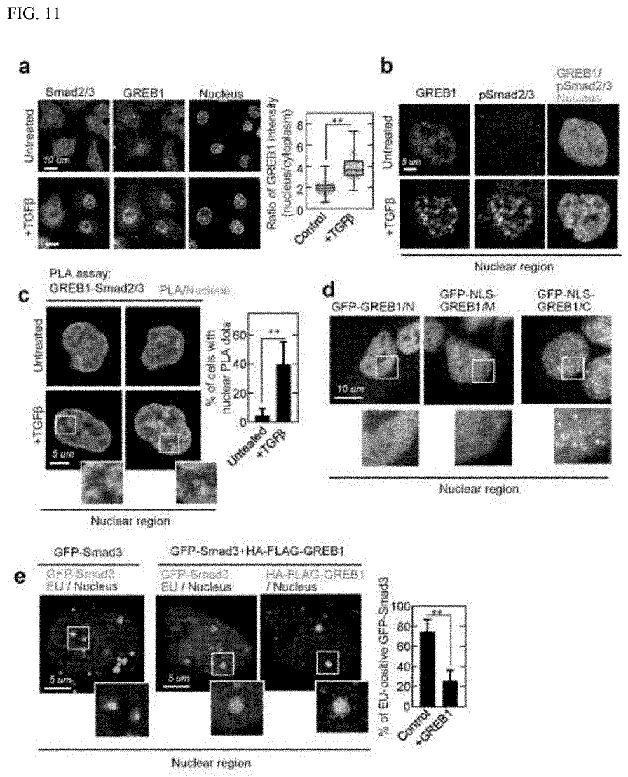

Further, HepG2 cells were cultured in the presence or absence of 10 ng/mL of TGFβ for 30 minutes, the cultured cells were fixed, and stained with an anti-SMAD2/3 antibody, anti-GREB antibody, and Hoechst33342. Thereafter, GREB1 fluorescence intensity in the nucleus and the cytoplasm was measured, and the results were expressed as ratios of GREB1 fluorescence intensity in the nucleus to GREB1 fluorescence intensity in the cytoplasm. As a result, it was demonstrated that endogenous GREB1 and Smad3 existed in both the cytoplasm and the nucleus, and they accumulated in the nucleus by stimulation with TGFβ (see a of FIG. 11).

Further, HepG2 cells were cultured in the presence or absence of 10 ng/mL of TGFβ for 30 minutes, and the cultured cells were fixed and stained with anti-GREB1 antibody, anti-phosphorylated SMAD2/3(pSMAD2/3) antibody, and Hoechst33342. As a result, it was confirmed that, in the cells treated with TGFβ, the phosphorylated SMAD2/3 accumulated in the nucleus, and colocalized with GREB1 (see b of FIG. 11).

Further, HepG2 cells were cultured in the presence or absence of 10 ng/mL of TGFβ for 30 minutes, and the cultured cells were fixed and incubated with mouse anti-GREB1 antibody and rabbit anti-SMAD2/3 antibody, which were added to the cultured cells. Next, to these primary antibodies, secondary antibodies (PLA (proximity ligation assay) probe) were allowed to bind. As a result, it was found that Smad2/3 localized TGFβ stimulation dependently in the vicinity of GREB1 in a boundary area between the chromatin region and the interchromatin region (see c of FIG. 11). GREB1 mutant (1 to 666 and NLS/667-1333) existed over the whole of the nucleus, but did not form nuclear focuses. On the contrary, GREB1 mutant (NLS/1334-1954) formed nuclear focuses as in GREB1 (full length). Accordingly, it is suggested that the C-terminal region of GREB1 has an important role in localization of GREB1 in a specific region in the nucleus (see d of FIG. 11).

To elucidate the functional interaction of specific localization of GREB1 and Smad2/3 in the nucleus, transcriptional activity was visualized by analysis of RNA synthesis using ethynyl uridine (EU). Specifically, first, HepG2 cells expressing GFP-SMAD3 with or without expression of HA-FLAG-GREB1 were incubated in the presence of EU at a final concentration of 1 mM for 30 minutes. The incubated cells were fixed, and stained with anti-GFP antibody, anti-FLAG antibody, and Hoechst33342. It has been confirmed that when HepG2 cells were incubated in the presence of EU for 30 minutes and the incubated cells were fixed, EU-labeled regions in the nucleus can be detected (see d of FIG. 10). As a result of the above-described experiment, in HepG2 cells expressing GFP-SMAD3, newly produced RNA molecules were observed over the whole of the nucleoplasm, and it was observed that some of the RNA molecules colocalized with nuclear focuses of GFP-Smad3 (see e of FIG. 11). On the other hand, in HepG2 cells expressing GFP-SMAD3 and HA-FLAG-GREB1, the EU label did not colocalize with nuclear focuses of GFP-Smad3 (see e of FIG. 11). That is, from these results, it is suggested that transcriptional activity associated with TGFβ-SMAD signal is selectively inhibited by interaction between Smad2/3 and GREB1 in a boundary area between a chromatin region and an interchromatin region.

### 2-6. GREB1 Participates in Formation of Hepatoblastoma-Like Tumor In Vivo

Some mouse liver tumor models of hepatocellular cancer and hepatoblastoma have been developed using genomic manipulation and/or hydrodynamic transfection of oncogenes. Further, it is reported that overexpression of constitutively active β-catenin and YAP using hydrodynamic transfection acutely induces liver tumors having characteristics of hepatocellular cancer and hepatoblastoma. In fact, when hepatoblastoma tissues (n=11) were immunostained with anti-β-catenin antibody and hematoxylin, tumor cell-specific overexpression of YAP in the cytoplasm or the nucleus in 9 tissues (positive rate: 81.8%) out of the 11 hepatoblastoma tissues were observed, and β-catenin and GREB1 were positive in all of the 9 tissues (see a of FIG. 12). Further, it is reported that HGF-c-Met pathway was activated in hepatoblastoma, and constitutively active form of coexpression of β-catenin and c-Met induces liver tumor in mouse.

A search for effective combinations of β-catenin, YAP, and c-Met for hepatoblastoma formation by a hydrodynamic transfection method was carried out. In a mouse transduced with both ΔN90 β-catenin, in which amino acids at positions 1 to 90 of human β-catenin were deleted, and YAPS127A, which was a YAP mutant in which serine at position 127 was substituted by alanine to avoid phosphorylation of the serine, in combination (BY model), a small tumor mass was formed in the liver 6 weeks after transduction, whereas almost no expression of GREB1 and DLK1 was observed immunohistologically (see b of FIG. 12). In a mouse transduced with ΔN90 β-catenin and c-Met in combination (BM model), small expression of GREB1 and DLK1 was observed immunohistologically in a tumor mass formed in the liver 6 weeks after transduction (see b of FIG. 12). On the other hand, in a mouse transduced with ΔN90 β-catenin, YAPS127A, and c-Met in combination (BYM model), large multiple tumor masses were formed in the liver 6 weeks after transduction, and high expressions of GREB1 and DLK1 were observed immunohistologically (see b of FIG. 12). Further, when mRNA levels of GREB1 and TACSTD1 in tumor nodules of BY model, BM model, and BYM model were measured by real-time PCR, the mRNA levels in BYM mouse were higher than those in BY model and BM model (see c of FIG. 12). From these results, BYM model is considered as a suitable model for in vivo functional analysis of GREB1 in hepatoblastoma.

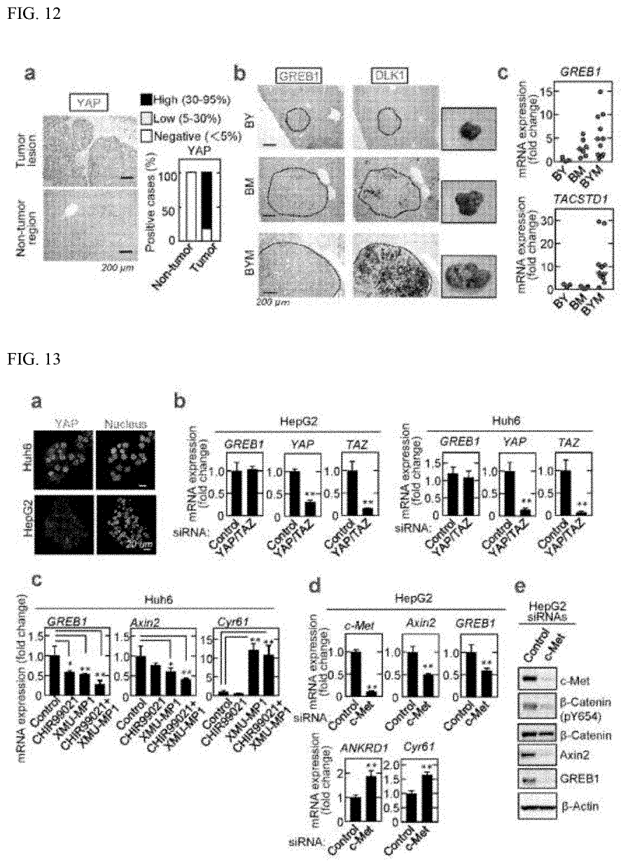

Next, the mechanism for regulating expression of GREB1 in hepatoblastoma by YAP and c-Met was investigated. Huh6 cells or HepG2 cells were fixed and stained with anti-YAP antibody and Hoechst33342, and it was found that YAP was strongly accumulated in the nucleus in Huh6 cells as compared to HepG2 cells (see a of FIG. 13). However, in Huh6 cells and HepG2 cells, knockdown of YAP/TAZ had no effect on expression of GREB1 (see b of FIG. 13). Further, when Huh6 cells was treated with CHIR99021, or treated with XMU-MP1, which has an inhibitory effect on Mst1/2 kinase and is an activator of YAP, or treated with CHIR99021 and XMU-MP1 in combination, mRNA expression of GREB1 was suppressed as in mRNA expression of Axin2 (see c of FIG. 13). From these results, it is suggested that YAP/TAZ is necessary for hepatoblastoma formation, but is not essential for GREB1 expression.

Further, when c-Met was knocked down in HepG2 cells, the expressions of Axin2 mRNA and GREB1 mRNA were suppressed, whereas the expressions of ANKRD1 and Cyr61, which are downstream target genes of YAP, were increased (see d of FIG. 13). It is reported that HGF-c-Met signaling pathway accelerates phosphorylation of tyrosine at position 654 of β-catenin and nuclear transport of β-catenin, and activates β-catenin signal. In fact, when c-Met was knocked down in HepG2 cells, phosphorylation of tyrosine at position 654 of β-catenin was weakened, and the expressions of GREB1 and Axin2 were decreased (see e of FIG. 13). From these results, it is suggested that c-Met induces phosphorylation of tyrosine of β-catenin, and thereby activates β-catenin signal to increase expression of GREB1.

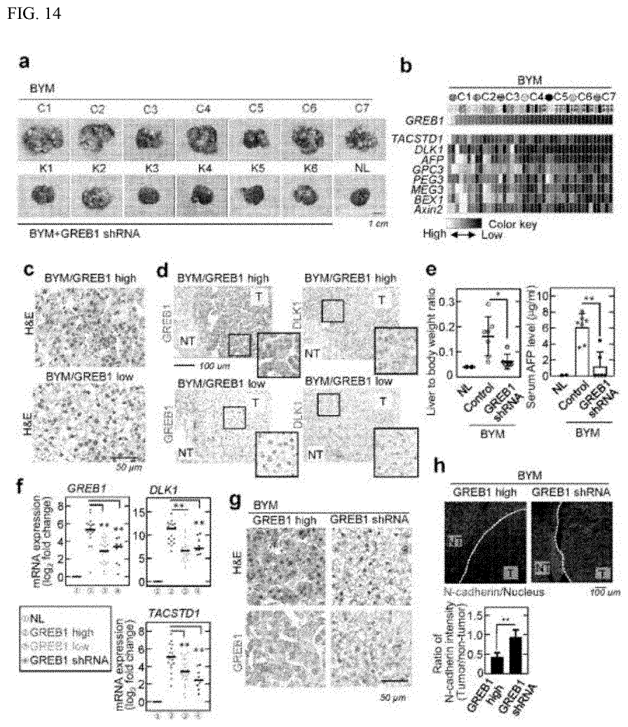

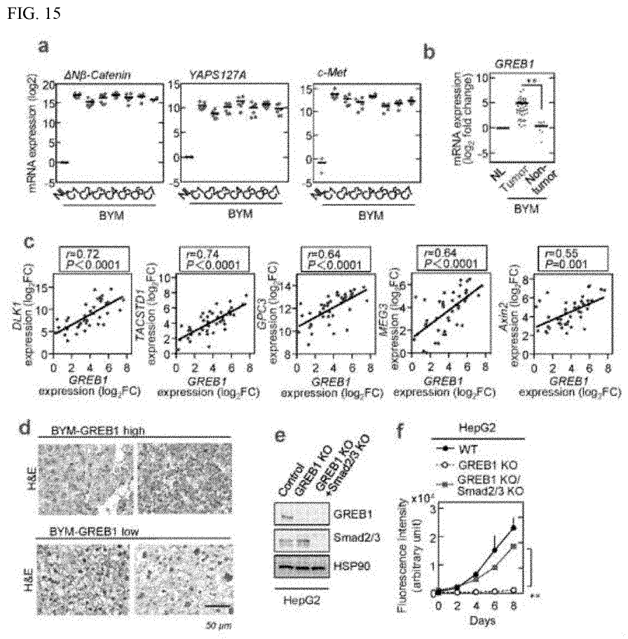

Further, ΔN90 β-catenin, YapS127A, and c-Met were administered to mice together with GREB1 shRNA, and the livers were collected 7 to 8 weeks after administration. As a result, in mice transduced with ΔN90 β-catenin, YAPS127A, and c-Met (BYM mice, control), a plurality of nodules were observed on the entire surfaces of the livers (see a of FIG. 14). On the other hand, in mice transduced with GREB1 shRNA together with ΔN90 β-catenin, YAPS127A, and c-Met (BYM GREB1 KD mice), the nodules in the liver were suppressed (see a of FIG. 14). In addition, when tumor nodules were obtained from the livers of BYM mice (C1 to C7) and the liver of an untreated mouse (NL mouse), and the expression levels of ΔN90 β-catenin, YAPS127A, and c-Met were measured by real-time PCR, it was demonstrated that all of the ΔN90 β-catenin, the YAPS127A, and the c-Met were expressed in BYM mice (see a of FIG. 15).

Further, total RNAs were extracted from the liver of an untreated mouse (NL), tumor nodules (n=42) of BYM mice, and nonneoplastic tissues (n=8) of BYM mice, and mRNA levels of GREB1 were measured by real-time PCR. As a result, mRNA levels of GREB1 in each of the tumor nodules were high as compared to those in the nonneoplastic tissues and the normal liver tissue (see b of FIG. 15).

Further, 6 tumor nodules were obtained from each of the BYM mice (C1 to C7), and the expressions of hepatoblastoma-related genes and histological appearances were investigated. As a result, by visualization using a heat map, it was demonstrated that the mRNA levels of GREB1 in the tumor nodules of the respective mice were different from each other (see b of FIG. 14). According to the mRNA levels, the mice were classified into two groups: a high GREB1 mRNA level group (GREB1 high: C1, C4, and C6) and a low group (GREB1 low: C2, C3, C5, and C7). The GREB1 high group had a tendency to express high TACSD1, DLK1 (hepatoblastoma marker gene), AFP, GFP3 (undifferentiated hepatoblast marker gene), PEG3, MEG3, BEX1 (imprinting genes), and Axin2 as compared to the GREB1 low group (see b of FIG. 14). Further, using tumor nodules obtained from the above-described BYM mice (C1 to C7), the expression levels of REB1, and mRNA expression levels of hepatoblastoma-related genes (DLK 1, TACSTD1, GPC3, MEG3, and Axin2) were measured, and it was demonstrated that there were strong positive correlations between the expression levels of GREB1 and the expression levels of hepatoblastoma-related genes (see c of FIG. 15, and Table 7).

TABLE 7
Analysis of correlation between hepatoblastoma markers

| | Epcam | GPC | AFP | Meg3 | Bex1 | Peg3 | Axin2 | Dlk1 |
|---|---|---|---|---|---|---|---|---|
| Greb1 | 0.75 | 0.65 | 0.70 | 0.55 | 0.68 | 0.40 | 0.55 | 0.77 |
| Epcam | | 0.60 | 0.47 | 0.34 | 0.58 | 0.31 | 0.48 | 0.43 |
| GPC | | | 0.81 | 0.60 | 0.76 | 0.39 | 0.62 | 0.62 |
| AFP | | | | 0.79 | 0.83 | 0.62 | 0.44 | 0.80 |
| Meg3 | | | | | 0.65 | 0.73 | 0.39 | 0.78 |
| Bex1 | | | | | | 0.71 | 0.33 | 0.69 |
| Peg3 | | | | | | | 0.13 | 0.55 |
| Axin2 | | | | | | | | 0.50 |

The values in the table are correlation coefficients.

Further, tissue sections of the livers resected from BYM mice in the GREB1 high group (C4) and the GREB1 low group (C3) were stained with hematoxylin-eosin. As a result, it was found that most tumors in the GREB1 high group had high nucleus/cytoplasm ratios, and had high proliferative capacity (see c of FIG. 14, and d of FIG. 15). Further, tumors in the GREB1 low group mainly contained large differentiated cells each having definite cytoplasm, a uniform round nucleus, and a small nucleolus (see c of FIG. 14, and d of FIG. 15).

Further, tissue sections of the livers resected from BYM mice in the GREB1 high group (C4) and the GREB1 low group (C3) were stained with anti-GREB1 antibody or anti-DLK1 antibody, and hematoxylin. As a result, it was demonstrated immunohistochemically that the expressions of GREB1 and DLK1 in the neoplastic lesion areas were high as compared to those of the nonneoplastic area, and the levels of staining was correlated with the levels of differentiation (see d of FIG. 14).

When GREB was knocked down in BYM mice (BYM GREB1 KD mice; K1 to K6), the incidence rate of tumorigenesis was decreased, and any tumor was not observed in 4 out of the 6 mice (see a of FIG. 14). Further, in BYM+ GREB1 shRNA mouse, the weight of the liver and serum AFP level were dramatically decreased as compared to those of BYM mouse without GREB knockdown (see e of FIG. 15).

Further, total RNAs were extracted from tumor nodules of three BYM mice (C1, C4, and C6), four BYM mice (C2, C3, C5, and C7), and two BYM mice dosed with GREB1 shRNA (BYM GREB1 KD mice; K2 and K4), and the mRNA levels of GREB1, DLK1, and TASCSTD1 were analyzed by real-time PCR. As a result, tumors of BYM GREB1 KD mice (K2 and K4) had low GREB1 mRNA expression levels, whereas the expression levels of DLK1 and TACSTD1 were the same as those of the GREB1 low group of BYM mice (see f of FIG. 14). Further, tissue sections of the livers of BYM mouse (C4) and BYM GREB1 KD mouse (K2) were stained with hematoxylin-eosin or anti-GREB1 antibody, and hematoxylin. As a result, it was demonstrated that these cells had well differentiated hepatoblastoma-like cells having definite cytoplasm (see g of FIG. 14).

Further, it is known that N-cadherin is a target gene of TGFβ signaling, and the expression level is high in hepatoblastoma cells, rather than in mesenchymal cells in tumor tissue. Thus, tissue sections of the livers of BYM mouse (C4) and BYM GREB1 KD mouse (K2) were stained with anti-N-cadherin antibody and Hoechst33342. As a result, in the BYM mouse, the expression of N-cadherin was low in neoplastic lesion areas as compared to that in nonneoplastic areas. On the other hand, in BYM GREB1 KD mice, it was found that N-cadherin was upregulated in neoplastic lesion areas, and the expression level was the same as that in nonneoplastic areas (see h of FIG. 14). That is, from these results, it is suggested that TGFβ signaling is activated by suppression of GREB1 expression.

From these results, it is revealed that, in this mouse model, GREB1 participates in hepatoblastoma-like histological pattern, marker gene expression, and tumorigenesis.

### 2-7. GREB1 can be Target Gene of Hepatoblastoma

To investigate whether or not GREB1 can be a target gene for treating hepatoblastoma, using CRISPR/Cas9 system, GREB1 knockout HepG2 cells (GREB1 KO cells), cells that were GREB1 knockout HepG2 cells allowed to express GREB1 (GREB1 KO/GREB1 cells), cells that were GREB1 knockout HepG2 cells transduced with GREB1ΔNLS (GREB1 KO/GREB1ΔNLS cells), and GREB1 and Smad2/3 knockout cells (GREB1 KO/Smad2/3 KO cells) were produced.

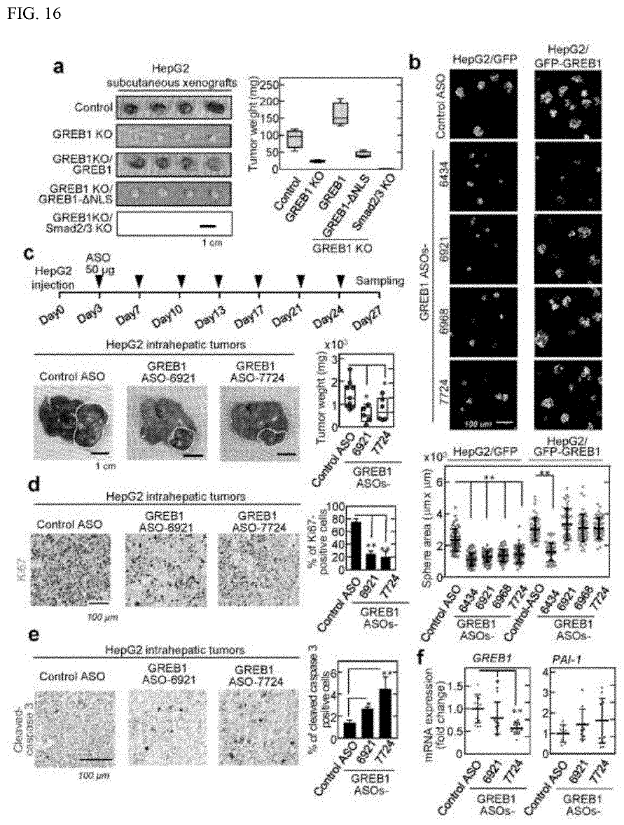

Wild type HepG2 cells (Control), GREB1 KO cells, GREB1 KO/GREB1 cells, GREB1 KO/GREB1ΔNLS cells, or GREB1 KO/Smad2/3 KO cells (7×10⁶ cells) were grafted into 5-week old male BALB/cAnNCrj-nu nude mice subcutaneously. The mice were sacrificed 28 days after grafting, and xenograft tumors were resected. The appearance and weight of the xenograft tumors were measured. As a result, subcutaneous xenograft tumors were formed in the case of wild type HepG2 cells, whereas the sizes and weights of tumors were decreased in GREB1 knockout HepG2 cells (see a of FIG. 16). Further, expression of GREB1 restored the phenotypes induced by GREB1 knockout, whereas the restoration was not observed by expression of GREB1ΔNLS (see a of FIG. 16). In addition, since knockout of Smad2/3 restored the phenotype induced by GREB1 knockout, it was confirmed that GREB1 inhibited the suppression of TGFβ signal-dependent cell proliferation in vivo.

Further, lysates of HepG2 cells (Control), GREB1 knockout HepG2 cells (GREB1 KO), or HepG2 cells in which GREB1 and Smad2/3 were knocked out in combination (GREB1 KO+Smad2/3 KO) were probed with anti-GREB1 antibody, anti-Smad2/3 antibody, and anti-HSP90 antibody. As a result, it was demonstrated that the protein expressions of GREB1 or Smad2/3 disappeared (see e of FIG. 15). In addition, two-dimensional culture (plastic dish culture) of HepG2 cells (WT), GREB1 knockout HepG2 cells (GREB1 KO), or HepG2 cells in which GREB1 and Smad2/3 were knocked out in combination (GREB1 KO/Smad2/3 KO) were performed, and the cell number was counted over time. As a result, since the suppression of cell proliferation by knockout of GREB1 was restored by knockout of Smad2/3, it is revealed that the phenotype is Smad2/3 dependent (see f of FIG. 15). From these results, it is also confirmed that GREB1 can inhibit the suppression of TGFβ signal-dependent cell proliferation in vivo.

### 2-8. Antisense Oligonucleotide Against GREB1 Decreases Proliferation of Hepatoblastoma Cells and Tumorigenesis

To elucidate the effect of an antisense oligonucleotide against GREB1 (GREB1 ASO) on proliferation of tumors, a base sequence of human GREB1 (15 nucleotides) as a target of GREB1 ASO was designed based on the secondary structure of GREB1 mRNA. Specifically, first, from thousands of candidate GREB1 ASOs, those which may have cytotoxicity were excluded, and twenty GREB1 ASO sequences were selected by higher order structure prediction. Based on these selected sequences, GREB1 ASOs with various modifications were designed and synthesized (Table 8).

TABLE 8

| | GREB1 | |
|---|---|---|
| No. | ASOs | Sequence (5′→3′) |
| 1 | hGREB1- | G(Y)\hat{ }A(Y)\hat{ }5(Y)\hat{ }a\hat{ }g\hat{ }g\hat{ }a\hat{ }a\hat{ }g |
| | 6424- | \hat{ }t\hat{ }a\hat{ }a\hat{ }A(Y)\hat{ }T(Y)\hat{ }c |
| | AmNA(15) | |
| 2 | hGREB1- | 5(Y)\hat{ }A(Y)\hat{ }G(Y)\hat{ }g\hat{ }a\hat{ }c\hat{ }a\hat{ }g\hat{ }g |
| | 6427- | \hat{ }a\hat{ }a\hat{ }g\hat{ }T(Y)\hat{ }A(Y)\hat{ }a |
| | AmNA(15) | |
| 3 | hGREB1- | G(Y)\hat{ }A(Y)\hat{ }A(Y)\hat{ }t\hat{ }g\hat{ }g\hat{ }c\hat{ }a\hat{ }g |
| | 6433- | \hat{ }g\hat{ }a\hat{ }c\hat{ }A(Y)\hat{ }G(Y)\hat{ }g |
| | AmNA(15) | |
| 4 | hGREB1- | 5(Y)\hat{ }G(Y)\hat{ }A(Y)\hat{ }a\hat{ }t\hat{ }g\hat{ }g\hat{ }c\hat{ }a |
| | 6434- | \hat{ }g\hat{ }g\hat{ }a\hat{ }5(Y)\hat{ }A(Y)\hat{ }g |
| | AmNA(15) | |
| 5 | hGREB1- | G(Y)\hat{ }5(Y)\hat{ }T(Y)\hat{ }a\hat{ }c\hat{ }a\hat{ }a\hat{ }a\hat{ }a |
| | 6500- | \hat{ }t\hat{ }a\hat{ }a\hat{ }5(Y)\hat{ }5(Y)\hat{ }c |
| | AmNA(15) | |
| 6 | hGREB1- | A(Y)\hat{ }A(Y)\hat{ }A(Y)\hat{ }t\hat{ }a\hat{ }c\hat{ }t\hat{ }g\hat{ }g |
| | 6513- | \hat{ }c\hat{ }a\hat{ }c\hat{ }5(Y)\hat{ }G(Y)\hat{ }c |
| | AmNA(15) | |
| 7 | hGREB1- | 5(Y)\hat{ }T(Y)\hat{ }A(Y)\hat{ }c\hat{ }t\hat{ }g\hat{ }a\hat{ }a\hat{ }a |
| | 6519- | \hat{ }t\hat{ }a\hat{ }c\hat{ }T(Y)\hat{ }G(Y)\hat{ }g |
| | AmNA(15) | |
| 8 | hGREB1- | T(Y)\hat{ }5(Y)\hat{ }5(Y)\hat{ }c\hat{ }a\hat{ }t\hat{ }c\hat{ }t\hat{ }a |
| | 6525- | \hat{ }c\hat{ }t\hat{ }g\hat{ }A(Y)\hat{ }A(Y)\hat{ }a |
| | AmNA(15) | |
| 9 | hGREB1- | A(Y)\hat{ }A(Y)\hat{ }G(Y)\hat{ }t\hat{ }a\hat{ }a\hat{ }g\hat{ }c\hat{ }a |
| | 6912- | \hat{ }a\hat{ }t\hat{ }g\hat{ }T(Y)\hat{ }G(Y)\hat{ }g |
| | AmNA(15) | |
| 10 | hGREB1- | G(Y)\hat{ }T(Y)\hat{ }5(Y)\hat{ }t\hat{ }g\hat{ }t\hat{ }t\hat{ }t\hat{ }c |
| | 6921- | \hat{ }a\hat{ }a\hat{ }g\hat{ }T(Y)\hat{ }A(Y)\hat{ }a |
| | AmNA(15) | |
| 11 | hGREB1- | T(Y)\hat{ }T(Y)\hat{ }5(Y)\hat{ }a\hat{ }t\hat{ }t\hat{ }g\hat{ }t\hat{ }c |
| | 6927- | \hat{ }t\hat{ }g\hat{ }t\hat{ }T(Y)\hat{ }T(Y)\hat{ }c |
| | AmNA(15) | |
| 12 | hGREB1- | T(Y)\hat{ }A(Y)\hat{ }T(Y)\hat{ }a\hat{ }t\hat{ }c\hat{ }a\hat{ }c\hat{ }t |
| | 6945- | \hat{ }t\hat{ }t\hat{ }g\hat{ }G(Y)\hat{ }T(Y)\hat{ }t |
| | AmNA(15) | |
| 13 | hGREB1- | T(Y)\hat{ }5(Y)\hat{ }T(Y)\hat{ }a\hat{ }g\hat{ }t\hat{ }t\hat{ }c\hat{ }t |
| | 6968- | \hat{ }c\hat{ }a\hat{ }t\hat{ }5(Y)\hat{ }A(Y)\hat{ }a |
| | AmNA(15) | |
| 14 | hGREB1- | A(Y)\hat{ }G(Y)\hat{ }T(Y)\hat{ }c\hat{ }a\hat{ }t\hat{ }a\hat{ }a\hat{ }g |
| | 6977- | \hat{ }t\hat{ }c\hat{ }t\hat{ }A(Y)\hat{ }G(Y)\hat{ }t |
| | AmNA(15) | |
| 15 | hGREB1- | T(Y)\hat{ }A(Y)\hat{ }5(Y)\hat{ }a\hat{ }g\hat{ }t\hat{ }c\hat{ }a\hat{ }t |
| | 6980- | \hat{ }a\hat{ }a\hat{ }g\hat{ }T(Y)\hat{ }5(Y)\hat{ }t |
| | AmNA(15) | |
| 16 | hGREB1- | G(Y)\hat{ }A(Y)\hat{ }G(Y)\hat{ }a\hat{ }a\hat{ }a\hat{ }a\hat{ }t\hat{ }g |
| | 7026- | \hat{ }a\hat{ }g\hat{ }c\hat{ }T(Y)\hat{ }A(Y)\hat{ }c |
| | AmNA(15) | |
| 17 | hGREB1- | T(Y)\hat{ }5(Y)\hat{ }T(Y)\hat{ }c\hat{ }t\hat{ }a\hat{ }g\hat{ }t\hat{ }c |
| | 7072- | \hat{ }a\hat{ }a\hat{ }g\hat{ }T(Y)\hat{ }G(Y)\hat{ }a |
| | AmNA(15) | |
| 18 | hGREB1- | T(Y)\hat{ }5(Y)\hat{ }5(Y)\hat{ }t\hat{ }c\hat{ }t\hat{ }c\hat{ }t\hat{ }a |
| | 7075- | \hat{ }g\hat{ }t\hat{ }c\hat{ }A(Y)\hat{ }A(Y)\hat{ }g |
| | AmNA(15) | |
| 19 | hGREB1- | G(Y)\hat{ }A(Y)\hat{ }G(Y)\hat{ }a\hat{ }a\hat{ }t\hat{ }g\hat{ }g\hat{ }t |
| | 7277- | \hat{ }g\hat{ }a\hat{ }g\hat{ }A(Y)\hat{ }A(Y)\hat{ }c |
| | AmNA(15) | |
| 20 | hGREB1- | A(Y)\hat{ }T(Y)\hat{ }T(Y)\hat{ }g\hat{ }a\hat{ }g\hat{ }q\hat{ }g\hat{ }t |
| | 7724- | \hat{ }a\hat{ }g\hat{ }g\hat{ }5(Y)\hat{ }A(Y)\hat{ }a |
| | AmNA(15) | |
| 21 | Control | T(Y)\hat{ }a\hat{ }g\hat{ }A(Y)\hat{ }g\hat{ }a\hat{ }G(Y)\hat{ }t\hat{ }a |
| | ASO | \hat{ }5(Y)\hat{ }c\hat{ }c\hat{ }A(Y)\hat{ }t\hat{ }c |

[In each sequence, “G(Y)” represents a guanine derivative having a structure of AmNA (2′,4′-bridged nucleotide having a structure represented by the above-described general formula (1) in which R is a methyl group), “A(Y)” represents an adenine derivative having a structure of AmNA, “T(Y)” represents a thymine derivative having a structure of AmNA, “5(Y)” represents a 5-methylcytosine derivative having a structure of AmNA, lower cases “a, t, c, g” each represent an unmodified DNA, and “\hat{ }” represents a phosphorothioate bond.]

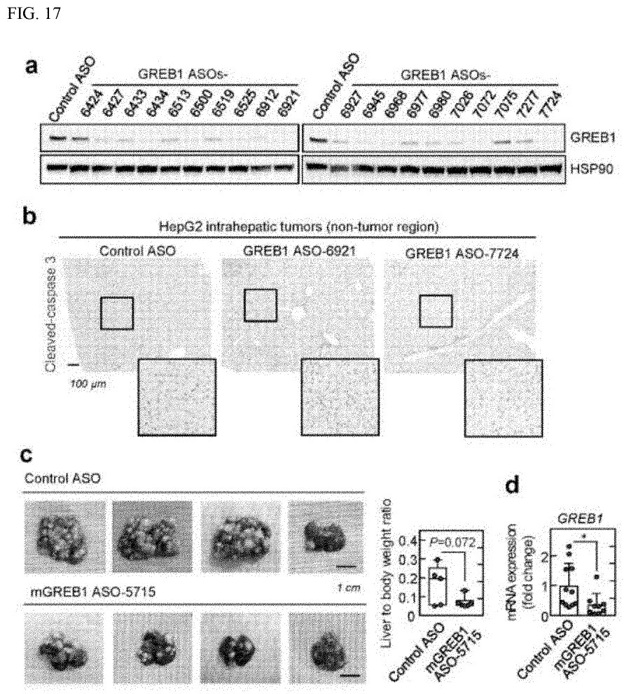

Next, HepG2 cells were transfected with control ASO or each of the above-described GREB1 ASOs, cultured in a medium containing 10% FBS for 2 days, and the cell lysates were probed with anti-GREB1 antibody and anti-HSP90 antibody. As a result, among twenty GREB1 ASOs, it was found that ASO-6434, ASO-6921, ASO-6968, and ASO-7724 did not have cytotoxicity, and strongly suppressed GREB1 expression in HepG2 cells (see a of FIG. 17).

Further, HepG2 cells expressing GFP or GFP-GREB1 were transfected with control ASO or each of the above-described GREB1 ASOs, and cultured in three-dimensional Matrigel for 4 days. The cultured cells were stained with phalloidin and Hoechst33342, and the areas of spheres were calculated (n=50). As a result, ASO-6434, ASO-6921, ASO-6968, and ASO-7724 were capable of preventing sphere-forming activity in HepG2 cells, whereas, in HepG2 cells expressing ASO resistant GREB1 (HepG2 cells expressing GFP-GREB1), the inhibitory effects on the sphere-forming activity of ASO-6921, ASO-6968, and ASO-7724 disappeared (see b of FIG. 16). From these results, it is revealed that at least ASO-6921, ASO-6968, and ASO-7724 prevent sphere formation of HepG2 cells by on-target effect (not by off-target effect).

At day 0, Matrigel containing HepG2 cells (1.0×10⁷ cells) were grafted into the livers of nude mice. Starting from day 3, 50 μg of control ASO (n=9), GREB1 ASO-6921 (n=5), or GREB1 ASO-7724 (n=6) were subcutaneously administered twice every week. At day 29 from grafting, the mice were euthanized, and the tumors were observed. As a result, in both GREB1 ASO-6921 and GREB1 ASO-7724, as compared to control ASO, tumorigenesis by HepG2 cells was suppressed, and the tumor weights were decreased (see c of FIG. 16). Further, mRNA levels of GREB1 and PAI-1 in tumors of each of the livers were analyzed by real-time PCR. In addition, sections of the tumors of each liver were stained with anti-Ki-67 antibody and hematoxylin, and the ratios of the number of Ki-67-positive cells to the number of hematoxylin-positive cells (total cells) were calculated. As a result, it was found that GREB1ASOs-6921 and -7724 suppressed GREB1 expression in tumors of the livers, and reduced the number of Ki-67-positive cells (see d of FIG. 16). Further, sections of the tumors of each liver were stained with anti-cleaved caspase 3 antibody and hematoxylin, and the ratios of the number of cleaved caspase 3-positive cells to the number of hematoxylin-positive cells (total cells) were calculated. As a result, it was found that apoptosis of tumor cells was induced by GREB1 ASOs-6921 and -7724 (see e of FIG. 16). In addition, when mRNA levels of GREB1 and PAI-1 in the tumors of each of the above-described livers were analyzed by real-time PCR, the PAI-1 gene expression in hepatoblastoma tumor demonstrated a tendency to be increased by GREB1 ASOs-6921 and -7724 (see f of FIG. 16), and it is suggested that proliferation and survival of tumor cells are suppressed by activation of TGFβ signaling. Further, sections of a nonneoplastic part of each liver were stained with anti-cleaved caspase 3 antibody and hematoxylin. As a result, it was demonstrated that GREB1 ASOs-6921 and -7724 did not induce histological damage or cell death in the nonneoplastic areas of the livers (see b of FIG. 17). From these results, it is revealed that an ASO against GREB1 can be a novel therapeutic drug of hepatoblastoma.

Further, mGREB1 ASO-5715 against mouse GREB1, and control ASO were prepared (see Table 9), and their effects on liver tumorigenesis in BYM model were analyzed. ΔN90 β-catenin, YAPS127A, and c-Met (BYM) were introduced into nude mice, and, starting 3 days after introduction, 50 μg of control ASO, and ASO against mouse GREB1 (mGREB1 ASO-5715) were subcutaneously administered twice every week. Appearance of the liver rumors 6 to 7 weeks after grafting was observed, and the ratio of the weight of the liver to the weight of whole body was analyzed. As a result, it was observed that the liver tumorigenesis had a tendency to be suppressed by administration of mGREB1 ASO-5715 (see c of FIG. 17). Further, when GREB1 mRNA levels in tumors of each of the above-described livers were analyzed by real-time PCR, it was demonstrated that the expressions of GREB1 in tumor tissues were suppressed in BMY mice dosed with mGREB1 ASO-5715 (d of FIG. 17).

TABLE 9

| Mouse GREB1 | |
|---|---|
| ASOs | Sequences (5′→3′) |
| mGREB1 | 5(Y)\hat{ }5(Y)\hat{ }G(Y)\hat{ }a\hat{ }g\hat{ }c\hat{ }a\hat{ }g\hat{ }g\hat{ } |
| ASO-5715 | g\hat{ }c\hat{ }a\hat{ }T(Y)\hat{ }A(Y)\hat{ }g |
| Control ASO | T(Y)\hat{ }a\hat{ }g\hat{ }A(Y)\hat{ }g\hat{ }a\hat{ }G(Y)\hat{ }t\hat{ }a\hat{ } |
| | 5(Y)\hat{ }c\hat{ }c\hat{ }A(Y)\hat{ }t\hat{ }c |

[In each scquence, “G(Y)” represents a guanine derivative having a structure of AmNA (2′,4′-bridged nucleotide having a structure represented by the above-described general formula (1) in which R is a methyl group), “A(Y)” represents an adenine derivative having a structure of AmNA, “T(Y)” represents a thymine derivative having a structure of AmNA, “5(Y)” represents a 5-methylcytosine derivative having a structure of AmNA, lower cases “a, t, c, g” each represent an unmodified DNA, and “\hat{ }” represents a phosphorothioate bond.]

### 2-9. GREB1 is Overexpressed in Neuroblastoma

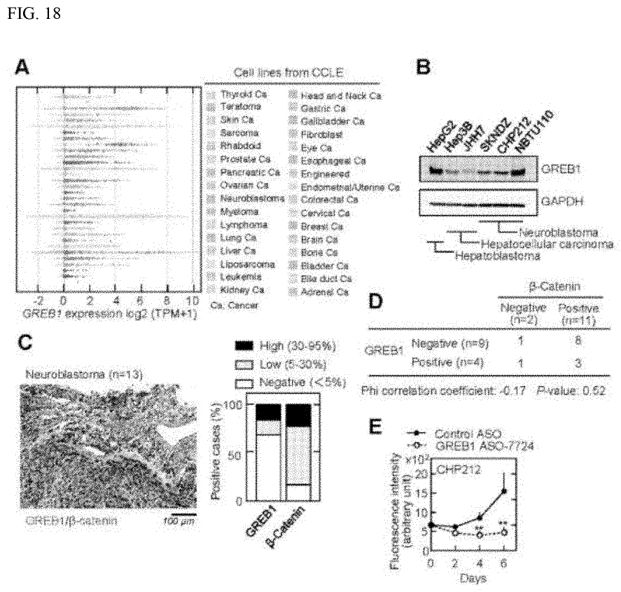

In each of the human cancer cell lines, mRNA expression of GREB1 was analyzed based on the dataset of The Cancer Cell Line Encyclopedia 1 (CCLE). GREB1 was highly expressed in, in addition to previously reported breast cancer cell lines, skin cancer (melanoma) cell lines, neuroblastoma cell lines, and some hepatocellular cancer cell lines (see A of FIG. 18). The expressions of GREB1 in SKNDZ cells, CHP212 cells, and NBTU110 cells, which are cells of neuroblastoma cell lines, were analyzed at protein level, and it was demonstrated that the expressions were the same as those in GREB1 high expression HepG2 cells, which are cells of a hepatoblastoma cell line, and higher than those in GREB1 high expression Hep3B cells and JHH7 cells, which are cells of hepatocellular cancer cell lines (see B of FIG. 18). Expressions of GREB1 and β-catenin in neuroblastoma tissues of 13 cases were analyzed immunohistologically. As described above, in hepatoblastoma, which is a childhood liver cancer, GREB1 is expressed as a downstream target gene of β-catenin. GREB1 was detected in the nucleus of each of the tumor cells in 4 cases of neuroblastoma (30.7%), and β-catenin was detected in the cytoplasm or the nucleus in 11 cases (84.6%) (see C of FIG. 18). No significant correlation between the expression of GREB1 and the expression of β-catenin was demonstrated (see D of FIG. 18). From these results, it is suggested that GREB1 is overexpressed in neuroblastoma cells independent of β-catenin.

In neuroblastoma cells, the influence of GREB1 on cell proliferation was investigated. In CHP212 cells, when expression of GREB1 was suppressed using an antisense oligonucleotide (ASO), cell proliferation under plate culture conditions was suppressed (see E of FIG. 18). From these results, it is revealed that GREB1 is important for cell proliferation in neuroblastoma.

### 2-10. GREB1 is Overexpressed in Some Hepatocellular Cancers, and Participates in Cell Proliferation and Tumorigenesis

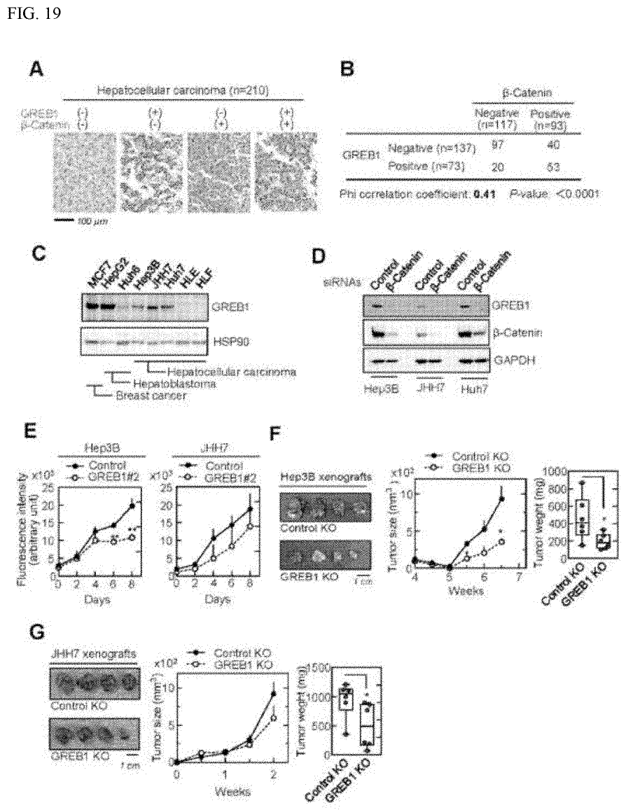

As described above, in hepatoblastoma, which is a childhood liver cancer, GREB1 is expressed as a downstream target gene of β-catenin. On the other hand, from the CCLE dataset, it is suggested that GREB1 is highly expressed also in some adult hepatocellular cancer (see A of FIG. 18). Accordingly, expressions of GREB1 and β-catenin in 210 cases of hepatocellular cancer tissues were analyzed immunohistologically. As a result, there were cases in each of which only GREB1 was highly expressed in the nucleus, cases in each of which only β-catenin was highly expressed in the nucleus or the cytoplasm, cases in which both GREB1 and β-catenin were highly expressed, or cases in each of which neither GREB1 nor β-catenin was expressed (see A of FIG. 19). GREB1 was detected in the nucleus of each of the tumor cells in 73 hepatocellular cancer cases (34.7%), and β-catenin was detected in the cytoplasm or the nucleus in 93 cases (44.2%) (see B of FIG. 19). Further, there was a significant positive correlation between the expression of GREB1 and the expression of β-catenin (phi correlation coefficient: 0.41, P value<0.0001) (see B of FIG. 19). In addition, the expressions of GREB1 in Hep3B cells, JHH7 cells, and Huh7 cells, in which expressions of GREB1 are high in the CCLE dataset, and HLE cells and HLF cells, in which expressions of GREB1 are low in the CCLE dataset, were analyzed by Western blotting. As a result, the expression level of GREB1 in Hep3B cells, JHH7 cells, and Huh7 cells, which are hepatocellular cancer cells, were somewhat lower than those in MCF7 cells, which are cells of a GREB1-positive breast cancer cell line, and HepG2 cells, which are cells of a hepatoblastoma cell line, whereas higher than those of Huh6 cells, which are cells of a GREB1 week expression hepatoblastoma cell line. On the other hand, in HLE cells and HLF cells, expression of GREB1 was not observed (see C of FIG. 19). In GREB1 high expression hepatocellular cancer cell lines, β-catenin-dependency of GREB1 was investigated. When the expressions of β-catenin were suppressed using siRNA in Hep3B cells, JHH7 cells, and Huh7 cells, the expressions of GREB1 were suppressed in all of these cells (see D of FIG. 19). From these results, it is shown that GREB1 is overexpressed β-catenin dependently in some hepatocellular cancer cells.

Next, in hepatocellular cancer cells, the influence of GREB1 on cell proliferation was investigated. In Hep3B and JHH7 cells, when expression of GREB1 was suppressed using siRNA, cell proliferation under plate culture conditions was suppressed (see E of FIG. 19). In addition, for the purpose of elucidating the involvement of GREB1 in in vivo tumorigenesis of hepatocellular cancer, GREB1 knockout Hep3B and JHH7 cells were produced, and xenograft subcutaneous tumorigenesis analysis was performed. As a result, in GREB1 knockout Hep3B cells and JHH7 cells, as compared to control cells, the tumor weights were significantly suppressed 6.5 weeks after grafting (Hep3B cells), and 2 weeks after grafting (JHH7 cells) (see F and G of FIG. 19). From these results, it is revealed that GREB1 plays an important role in cell proliferation of hepatocellular cancer and in vivo tumorigenesis.

### 2-11. GREB1 is Overexpressed in Cutaneous Melanoma and Participates in Cell Proliferation

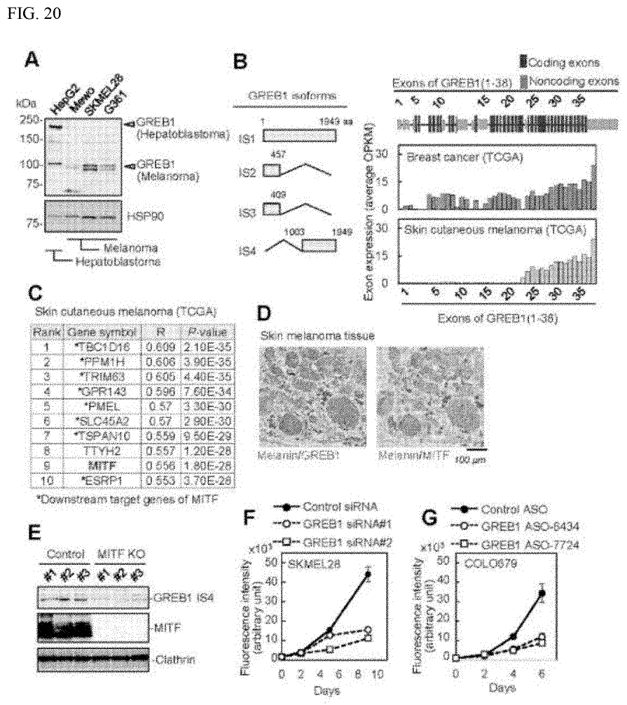

From the CCLE dataset, it is suggested that GREB1 is highly expressed at a high frequency in cutaneous melanoma cells (see A of FIG. 18). Accordingly, the expressions of GREB1 in Mewo cells, SKMEL28 cells, and G361 cells, which are melanoma cell lines in which the expressions of GREB1 are high in the CCLE dataset, were analyzed by Western blotting. As a result. GREB1 was detected around 216 kDa, which corresponds to wild type, in hepatoblastoma cells HepG2 cells, whereas those in all of the melanoma cells were detected around 100 kDa (see A of FIG. 20). Therefore, to elucidate the expressions of transcriptional variants of GREB1, based on the TCGA dataset, exon expressions of GREB1 in breast cancer and cutaneous melanoma were investigated using TCGA SpliceSeq (http://projects.insilico.us.com/TCGASpliceSeq). Four isoforms (isoforms; IS) (transcriptional variants) of GREB1 are registered on The Universal Protein Resource (Uniprot). IS1 is also referred to as GREB1a, and is the full-length form having 1949 amino acids (216 kDa); IS2 is also referred to as GREB1b, and is a transcript, in which amino acids in the C-terminal region at positions 458 to 1949 are deleted, having 457 amino acids (49 kDa); IS3 is also referred to as GREB1c, and is a transcript, in which amino acids in the C-terminal region at positions 410 to 1949 are deleted, having 409 amino acids (43 kDa); and IS4 is a transcript, in which amino acids in the N-terminal region at positions 1 to 1002 are deleted, having 947 amino acids (107 kDa) (see B of FIG. 20). GREB1 consists of 38 exons, and expressions of whole of the protein coding exons (exons 4 to 38) were observed in breast cancer tissues. On the other hand, it was demonstrated that, in cutaneous melanoma, GREB1 was expressed starting from exon 24, and IS4 was specifically expressed (see B of FIG. 20). On the TCGA, cancers expressing IS4 are merely cutaneous and ocular melanomas (data not shown).

To elucidate the mechanism for regulating expression of GREB1 in cutaneous melanoma, a group of genes having positive correlations with GREB1 in melanomas was identified using the TCGA dataset. Among the genes in the group, top 10 genes having higher correlation coefficients include MITF, and 8 known downstream target genes of MITF (see C of FIG. 20). MITF is a transcription factor that regulates the expressions of melanin biosynthesis system enzyme genes, and regulates differentiations of melanocyte, or retinal pigment epithelial cells (RPE), and the like. When the expressions of GREB1 and MITF in human melanoma tissues were analyzed immunohistologically, GREB1 and MITF were coexpressed in the same tumor regions in serial specimens (see D of FIG. 20). From this result, it is suggested that GREB1 can be a target gene of MITF in melanoma. Accordingly, in COLO679 cells, which are cells of GREB1 high expression melanoma cell line, when MITF knockout cells were produced, expression of GREB1 was reduced as compared to that in control cells (see E of FIG. 20).

In cutaneous melanoma cells, the influence of GREB1 on cell proliferation was investigated. In SKMEL28 cells and COLO679 cells, when expression of GREB1 was suppressed using siRNA (SKMEL28 cells) or an antisense oligonucleotide (ASO) (COLO679 cells), cell proliferation under plate culture conditions was suppressed in both cases (see F and G of FIG. 20). From these results, it is revealed that GREB1 IS4 that specifically expressed in cutaneous melanoma is important for cell proliferation of melanoma.

1. A therapeutic agent for a GREB1-positive tumor that does not show sex hormone sensitivity, comprising a substance that suppresses expression of GREB1 as an active ingredient.

2. The therapeutic agent according to claim 1, wherein the substance is at least one nucleic acid drug selected from the group consisting of siRNA, shRNA, dsRNA, an antisense nucleic acid, and ribozyme against GREB1.

3. The therapeutic agent according to claim 1, wherein the tumor is hepatoblastoma, hepatocellular cancer, malignant melanoma, or neuroblastoma.

4. A testing method for estimating whether or not a subject is affected with hepatoblastoma, hepatocellular cancer, malignant melanoma, or neuroblastoma, the method comprising a step of measuring expression level of GREB1 in a liver tissue, a skin tissue, or a nerve tissue sampled from the subject.

5. The testing method according to claim 4, wherein the expression level of GREB1 in a liver tissue, a skin tissue, or a nerve tissue is measured by immunohistochemical analysis using an anti-GREB1 antibody.

6. A testing agent for hepatoblastoma, hepatocellular cancer, malignant melanoma, or neuroblastoma, comprising an agent for detecting GREB1.

7. The testing agent according to claim 6, wherein the agent for detecting GREB1 is an anti-GREB1 antibody or a fragment thereof, or a primer capable of hybridizing to GREB1 mRNA or GREB1 cDNA.

8. A therapeutic method for treating a GREB1-positive tumor that does not show sex hormone sensitivity, comprising administering a therapeutically effective amount of a substance that suppresses expression of GREB1 to a patient having the GREB1-positive tumor that does not show sex hormone sensitivity.

9. The therapeutic method according to claim 8, wherein the tumor is hepatoblastoma, hepatocellular cancer, malignant melanoma, or neuroblastoma.

10.-13. (canceled)


SEQUENCE LISTING (109 sequences)

SEQ ID NO: 1 - protein, 1949 aa, Homo sapiens
MGNSYAGQLKTTRFEEVLHNSIEASLRSNNLVPRPIFSQLYLEAEQQLAALEGGSRVDNEEEEEEGEGGLETNGPPNPFQLHPLPEGCCTTDGFCQAGKDLRLVSISNEPMDVPAGFLLVGVKSPSLPDHLLVCAVDKRFLPDDNGHNALLGFSGNCVGCGKKGFCYFTEFSNHINLKLTTQPKKQKHLKYYLVRNAQGTLTKGPLICWKGSEFRSRQIPASTCSSSLFPALESTAAFPSEPVPGTNPSILMGAQQAGPASDHPSLNAAMGPAVFNGKDSPKCQQLAKNNLLALPRPSALGILSNSGPPKKRHKGWSPESPSAPDGGCPQGGGNRAKYESAGMSCVPQVGLVGPASVTFPVVASGEPVSVPDNLLKICKAKPVIFKGHGNFPYLCGNLNDVVVSPLLYTCYQNSQSVSRAYEQYGASAIQPISEEMQLLLTVYYLVQLAADQVPLMEDLEQIFLRSWRESHLTEIRQYQQAPPQPFPPAPSAAAPVTSAQLPWLASLAASSCNDSVHVIECAYSLAEGLSEMFRLLVEGKLAKTNYVVIICACRSAAIDSCIAVTGKYQARILSESLLTPAEYQKEVNYELVTGKVDSLGAFFSTLCPEGDIDILLDKFHQENQGHISSSLAASSVTKAASLDVSGTPVCTSYNLEPHSIRPFQLAVAQKLLSHVCSIADSSTQNLDLGSFEKVDFLICIPPSEVTYQQTLLHVWHSGVLLELGLKKEHMTKQRVEQYVLKLDTEAQTKFKAFLQNSFQNPHTLFVLIHDHAHWDLVSSTVHNLYSQSDPSVGLVDRLLNCREVKEAPNIVTLHVTSFPYALQTQHTLISPYNEIHWPASCSNGVDLYHENKKYFGLSEFIESTLSGHSLPLLRYDSSFEAMVTALGKRFPRLHSAVIRTFVLVQHYAAALMAVSGLPQMKNYTSVETLEITQNLLNSPKQCPCGHGLMVLLRVPCSPLAVVAYERLAHVRARLALEEHFEIILGSPSSGVTVGKHFVKQLRMWQKIEDVEWRPQTYLELEGLPCILIFSGMDPHGESLPRSLRYCDLRLINSSCLVRTALEQELGLAAYFVSNEVPLEKGARNEALESDAEKLSSTDNEDEELGTEGSTSEKRSPMKRERSRSHDSASSSLSSKASGSALGGESSAQPTALPQGEHARSPQPRGPAEEGRAPGEKQRPRASQGPPSAISRHSPGPTPQPDCSLRTGQRSVQVSVTSSCSQLSSSSGSSSSSVAPAAGTWVLQASQCSLTKACRQPPIVFLPKLVYDMVVSTDSSGLPKAASLLPSPSVMWASSFRPLLSKTMTSTEQSLYYRQWTVPRPSHMDYGNRAEGRVDGFHPRRLLLSGPPQIGKTGAYLQFLSVLSRMLVRLTEVDVYDEEEININLREESDWHYLQLSDPWPDLELFKKLPFDYIIHDPKYEDASLICSHYQGIKSEDRGMSRKPEDLYVRRQTARMRLSKYAAYNTYHHCEQCHQYMGFHPRYQLYESTLHAFAFSYSMLGEEIQLHFIIPKSKEHHFVFSQPGGQLESMRLPLVTDKSHEYIKSPTFTPTTGRHEHGLFNLYHAMDGASHLHVLVVKEYEMAIYKKYWPNHIMLVLPSIFNSAGVGAAHFLIKELSYHNLELERNRQEELGIKPQDIWPFIVISDDSCVMWNVVDVNSAGERSREFSWSERNVSLKHIMQHIEAAPDIMHYALLGLRKWSSKTRASEVQEPFSRCHVHNFIILNVDLTQNVQYNQNRFLCDDVDFNLRVHSAGLLLCRFNRFSVMKKQIVVGGHRSFHITSKVSDNSAAVVPAQYICAPDSKHTFLAAPAQLLLEKFLQHHSHLFFPLSLKNHDHPVLSVDCYLNLGSQISVCYVSSRPHSLNISCSDLLFSGLLLYLCDSFVGASFLKKFHFLKGATLCVICQDRSSLRQTVVRLELEDEWQFRLRDEFQTANAREDRPLFFLTGRHI

SEQ ID NO: 2 - RNA, 8555 nt, Homo sapiens
gcuaaaugguuaagagauacauaaauacugcaguagagaugggauuuuaccucaaagugcaaagguaaaugaaauaaaguuuuuucaauggaaggcuugcagcucuugaggaccugccaaauggaagaaggacagagaccuggagcccuauggaaaguucugacaccauguguggaaggacauggcuuuuaacacguguggugacuggaguagcugcagcugaggacagccacccuuucuucgucucugcugagcgaaggcuacacggcccuuccuccuugcagcuguuucaccuucuaccuugcguggagccaggcuuuugcaccgaaucugagaugccauuuuaaacagaagacuccauccucuugaagaugggaaauucuuacgcuggacagcugaagacgacacgcuuugaagaggucuugcacaauuccaucgaggcaucccugcgguccaacaaccuggugcccaggcccaucuuuucccagcuguaccuggaagcugagcagcagcuugccgcucuagaaggugguagccgaguggacaaugaggaagaggaagaagagggagaaggagggcuggaaacaaauggccccccaaacccuuuccagcugcacccucugccugaaggaugcuguaccacagacggguuuugccaggccgggaaggaccugcgccuugucuccauuuccaacgagcccauggaugucccugcgggcuuucuccucgugggggucaagucccccagccugccggaccaucuccuggugugcgccguugacaagagguucuugccagaugacaauggccacaaugcucuucuugguuucucugggaauuguguuggcuguggaaagaaaggcuucuguuacuucacggaauucuccaaucauauaaaucugaaacugaccacucaacccaagaagcagaaacacuugaaguauuaccugguccguaaugcacaagggacucuaaccaaaggaccuuuaaucuguuggaaaggcucagaguuuagaagccggcagauccccgccaguacuuguuccaguucccucuucccagcccuggagagcacggcugccuuccccagcgagcccguuccugggacgaaccccagcauccugaugggagcucagcaggcaggaccagcuucugaucaccccucacuaaacgcagcaauggguccggcuguuuucaacggcaaagauuccccgaagugccaacaacuggcaaagaauaaccuguuggcccugccgcgaccaucggcuuuagguaucuugucaaacuccgggccccccaaaaaacgccacaaaggguggucuccagaaucuccaucagcuccagaugguggcugcccccaagguggugggaacagagcuaaguaugagagcgcaggcauguccugcgugccgcagguuggcuuggugggaccagcuucagucaccuuuccagugguggccucuggagaaccagugucuguuccugacaacuugcugaaaauaugcaaggccaagccagugauauuuaaaggccaugggaacuucccuuaccucugugggaaccugaaugacgucguggucagcccccucuuguacacgugcuaccagaauucccagucugucucacgggcauacgagcaguacggcgccucugccauccagcccaucuccgaggagaugcagcuccugcuuaccgucuacuaccugguccagcuggccgcggaccaggugcccuugauggaggaccuggagcagaucuuccugcgcucuuggcgcgagucgcaccugaccgagauccggcaguaccagcaggcgccgccgcagcccuucccgcccgcgcccagcgccgcggcacccgugaccuccgcgcagcugcccuggcuggccagccuggccgccagcuccugcaacgacagcgugcacgucaucgagugugcuuacucccuggccgagggccucuccgagauguuccggcuguuggucgagggcaagcuugccaagaccaacuacguggucaucaucugcgccugccgcagcgcggccaucgacuccugcaucgccgucaccggaaaauaccaagcccggauucuuuccgagagccuucucacuccugcggaguaccagaaggaagucaauuacgagcugguuacggggaagguagacucgcugggggccuucuuuagcacccucuguccagagggugacauugacauuuugcuggacaaauuucaccaggaaaaucaaggccauauuucuuccucacucgcugccucuucugucacuaaagcagcaucccuggaugucagugggacaccggugugcacaaguuacaaucuggagccacacagcauccggcccuuccagcuggcaguagcgcagaagcuccucucccauguguguuccauugcggauuccagcacccaaaaucuggaccugggauccuuugagaagguggacuuucucauuugcauuccccccucagaagugaccuaccagcagacucugcuccauguguggcauucagggguuuugcuggagcuuggucugaagaaagagcacaugacgaagcagaggguggaacaguauguucugaagcuagacacggaggcacagacaaaauuuaaggcuuuucugcaaaacuccuuccagaacccgcauacacuuuuuguccuaauccaugaccaugcgcacugggaucuugugaguagcacuguucauaaccucuauucucaaagugacccgucggugggauugguggaccgauugcucaacugcagggaggugaaggaggcccccaacauugugacacuucacgugaccuccuucccguaugcacugcagacacagcacacccucaucagccccuacaacgagauccacuggccugccuccugcaguaauggaguggacuuauaucaugaaaauaagaaguacuucgggcugucggaguuuauugaauccacccuuucaggacacagccuccccuugcucagauacgauagcuccuuugaggccauggucacugcauuaggaaaaagguucccccgccugcacagcgcggugaucaggaccuuuguucucgugcagcacuacgcggccgcccugauggccguaagcggccucccgcagaugaagaacuacacgucgguggagacgcuggagaucacgcagaaccuccucaacuccccgaagcagugccccugcggccacgggcucaugguccugcugcgggugcccuguucgccccuggcggugguggccuaugagcggcuggcccacgugcgggcccggcuggcgcuggaggagcacuuugagaucauccugggcagucccagcucaggcgucaccguggggaagcacuucguaaagcagcucaggauguggcagaaaauugaggauguggaguggagaccccagacuuacuuggagcuggagggucugccuugcauccugaucuucagugggauggacccgcauggggaguccuugccgaggucuuugagguacugugaccugcgauugauaaacuccuccugcuuggugagaacagccuuggagcaggagcugggccuggcugccuacuuugugagcaacgagguucccuuggagaagggggcuaggaacgaggccuuggagagugaugcugagaagcugagcagcacagacaacgaggaugaggagcuggggacagaaggcucuaccucggagaagagaagccccaugaaaagggagaggucccgcucccacgacucagcauccucaucccucuccuccaaggcuuccgguucagcgcucgguggcgaguccucggcucagcccacagcacucccccagggagagcaugccaggucgccccagccccguggccccgcagaggagggcagagccccuggugagaaacagaggccccgggcaagucaggggccacccucggccaucagcaggcacagucccgggccgacgccccagcccgacuguagccucaggaccggccagaggagcguccaggugucggucaccucgucgugcucccagcuguccuccuccucgggcucauccuccucauccguggcgcccgcugccggcacguggguccugcaggccucccagugcuccuugaccaaggccugccgccagccacccauugucuucuugcccaagcucguguacgacaugguuguguccacugacagcaguggccugcccaaggccgccucccuccugcccucccccucggucaugugggccagcucuuuccgcccccugcucagcaagaccaugacauccaccgagcagucccucuacuaccggcaguggacggugccccggcccagccacauggacuacggcaaccgggccgagggccgcguggacggcuuccacccccgcaggcugcugcucagcggccccccucagaucgggaagacaggugccuaccugcaguuccucaguguccuguccaggaugcuuguucggcucacagaaguggaugucuaugacgaggaggagaucaauaucaaccucagagaagaaucugacuggcauuaucuccagcuuagcgaccccuggccagaccuggagcuguucaagaaguugcccuuugacuacaucauucacgacccgaaguaugaagaugccagccugauuuguucgcacuaucaggguauaaagagugaagacagagggaugucccggaagccggaggaccuuuaugugcggcgucagacggcacggaugagacuguccaaguacgcagcguacaacacuuaccaccacugugagcagugccaccaguacaugggcuuccacccccgcuaccagcuguaugaguccacccugcacgccuuugccuucucuuacuccaugcuaggagaggagauccagcugcacuucaucauccccaaguccaaggagcaccacuuugucuucagccaaccuggaggccagcuggagagcaugcgacuaccccucgugacagacaagagccaugaauauauaaaaaguccgacauucacuccaaccaccggccgucacgaacaugggcucuuuaaucuguaccacgcaauggacggugccagccauuugcacgugcugguugucaaggaauacgagauggcaauuuauaagaaauauuggcccaaccacaucaugcuggugcuccccaguaucuucaacagugcuggaguuggugcugcucauuuccucaucaaggagcuguccuaccauaaccuggagcucgagcggaaccggcaggaggagcugggaaucaagccgcaggacaucuggccuuucauugugaucucugaugacuccugcgugauguggaacgugguggaugucaacucugcuggggagagaagcagggaguucuccuggucggaaaggaacgugucuuugaagcacaucaugcagcacaucgaggcggcccccgacaucaugcacuacgcccugcugggccugcggaagugguccagcaagacccgggccagcgaggugcaagagcccuucucccgcugccacgugcacaacuucaucauccugaacguggaccugacccagaacgugcaguacaaccagaaccgguuccugugugacgauguagacuucaaccugcgggugcacagcgccggccuccugcucugccgguucaaccgcuucagcgugaugaagaagcagaucguggugggcggccacagguccuuccacaucacauccaaggugucugauaacucugccgcggucgugccggcccaguacaucugugccccggacagcaagcacacguuccucgcagcgcccgcccagcuccugcuggagaaguuccugcagcaccacagccaccucuucuucccgcugucccugaagaaccaugaccacccagugcugucugucgacuguuaccugaaccugggaucucagauuucuguuugcuaugugagcuccaggccccacucuuuaaacaucagcugcucggacuugcuguucagugggcugcugcuguaccucugugacucuuuugugggagcuagcuuuuugaaaaaguuucauuuucugaaaggugcgacguugugugucaucugucaggaccggagcucacugcgccagacggucguccgccuggagcucgaggacgaguggcaguuccggcugcgcgaugaguuccagaccgccaaugccagggaagaccggccgcucuuuuuucugacgggacgacacaucugaggaagacagcggcgaguuuucugaagagaugagugcucagagcccucaugcuguugaggcuaaagggaggccuggaacgguggggcguuugacuggaauggaccccagggacuguccaggugcagccccuccuaguacacaugggcccccgaggccgugguccugggagccaggaagacuccgcagugggugagaaugaaaacuugagacucccaaguucugggccagcccauugcucugggcuguuuuaaagcccauuucacgaggaacaaagauuuacuuccuguccugccauucgugugcuuccauggacaaaccugauuuuuuucucuuaguucuaaagaaucuuggguuauuuuguagcggugccaguauuucaguagaugggauuucagccaaguagguuccccuguaaccuccuacaaagcaauauuccaaaggaacauuuuaacuguaaaggcuggagacaagaaaaaauaaguagaucguuuuaauaacaauuauuuaauugccuauaaguuugcuguuucagaggcuagcccaaaggcaucaaauuuaauaaaguuaaacaaauugauuuacuucagagcaaauaugauccuauuaaaauaauauaggguaaauacccuaccucuuagaaagggcaaaaaugcaaagaagcuuucuuuaaaacuaaaaggguuuuuuggggggggaguuggcggggaggaaauaaggcuaacagagguugaccuaaaauuagccuuacaaaggagaaaggaccacauugcuuacuugaaacagacaaugaaaacaaccaaagugauauauaaaauaguugaugagaacuagacuuaugacuguaguuuacuagaguuuaguuuucaguugcugaaguagcucauuuucucuuacuaauguuugguuccucagggaagaaucucacuugacuagagaggaggugggaacagaagagagaaggaggcagggagauguauuucuuagggcucaccccuucacagacugacagaaugguuuuguuuuguuuuguuuuguuuuguuuuguuuuugagauggacucuagcucugucacccaggcuggagugcaguggugcgaucucggcucacugcaagcuccgccucccggguucucaccauucuccugccucagccucccgaguagcugggacuacaggcgcccaccaccacgcccggcuaauuuuuuguauuuuuuaguagagacgggguuucaccauguuagccaggauggucucgaucuccugaccucgugauccgcccgccucggccucccaaagugcugggauuacaggcgugagccaccgugccugccccagaaugguuuuuaaagccacaguugagaggccacccauugcccggcgccuggacagugaucaucuuguucaucuuguucaguccuuucuugugugauuggaauuauucauccccuuugaaagaugagaagguugagaugcaaagagucuaccuuuccaaguucucacugcuggaaagagcuagaagcacaguucaaaguucuggcuucuggacucugcaguccaggucucccuucucccacuugccuacccucaaugccacacuguuuuugaaguggcccauaacuugaaggaaaaguuuaaagacaguucaauuuaaucaucagaaugcauucuuuuuuuuuucggagacggaguuucacucuugcugcccaggcuggagugcaauggugcaaugaccucggcucacugcaaccucugccuccuggguucaagugauucuccagccucagccucccgaguagcugggauuaugggcgcccaccaccaugcccagcuaauuuuuguauuuuuuuuuuuuaguagagaugggguuucgccagguuggccaggcuggucuugugaacuccuggccucaggugaucugcccaccucauccuccaaaagugcugggauuacaggcaugagccacugcgccuggccucagaaugcauucuuacacaucuauccuagacauuuauaagcacucuaauggauaacaauccaagaauaaaugauuguaaaagaugaugccgaagaguugaugucaaucuuuuuuuccuaagaaaaaaaguccgcgaguauuaaauauuuagaucaauguuuauaaaaugauuacuuuguauaucucauuauuccuauuuuggaauaaaaacugaccuucuuuaaucauauacuugucuuuuguaaauagcagcuuuugugucauucuccccacuuuauuaguuaauuuaaauuggaaaaaacccucaaacuaauauucuugucuguuccagucuuauaaauaaaacuuauaaugcaugua

SEQ ID NO: 3 - DNA, 5850 bp, Homo sapiens
atgggaaattcttacgctggacagctgaagacgacacgctttgaagaggtcttgcacaattccatcgaggcatccctgcggtccaacaacctggtgcccaggcccatcttttcccagctgtacctggaagctgagcagcagcttgccgctctagaaggtggtagccgagtggacaatgaggaagaggaagaagagggagaaggagggctggaaacaaatggccccccaaaccctttccagctgcaccctctgcctgaaggatgctgtaccacagacgggttttgccaggccgggaaggacctgcgccttgtctccatttccaacgagcccatggatgtccctgcgggctttctcctcgtgggggtcaagtcccccagcctgccggaccatctcctggtgtgcgccgttgacaagaggttcttgccagatgacaatggccacaatgctcttcttggtttctctgggaattgtgttggctgtggaaagaaaggcttctgttacttcacggaattctccaatcatataaatctgaaactgaccactcaacccaagaagcagaaacacttgaagtattacctggtccgtaatgcacaagggactctaaccaaaggacctttaatctgttggaaaggctcagagtttagaagccggcagatccccgccagtacttgttccagttccctcttcccagccctggagagcacggctgccttccccagcgagcccgttcctgggacgaaccccagcatcctgatgggagctcagcaggcaggaccagcttctgatcacccctcactaaacgcagcaatgggtccggctgttttcaacggcaaagattccccgaagtgccaacaactggcaaagaataacctgttggccctgccgcgaccatcggctttaggtatcttgtcaaactccgggccccccaaaaaacgccacaaagggtggtctccagaatctccatcagctccagatggtggctgcccccaaggtggtgggaacagagctaagtatgagagcgcaggcatgtcctgcgtgccgcaggttggcttggtgggaccagcttcagtcacctttccagtggtggcctctggagaaccagtgtctgttcctgacaacttgctgaaaatatgcaaggccaagccagtgatatttaaaggccatgggaacttcccttacctctgtgggaacctgaatgacgtcgtggtcagccccctcttgtacacgtgctaccagaattcccagtctgtctcacgggcatacgagcagtacggcgcctctgccatccagcccatctccgaggagatgcagctcctgcttaccgtctactacctggtccagctggccgcggaccaggtgcccttgatggaggacctggagcagatcttcctgcgctcttggcgcgagtcgcacctgaccgagatccggcagtaccagcaggcgccgccgcagcccttcccgcccgcgcccagcgccgcggcacccgtgacctccgcgcagctgccctggctggccagcctggccgccagctcctgcaacgacagcgtgcacgtcatcgagtgtgcttactccctggccgagggcctctccgagatgttccggctgttggtcgagggcaagcttgccaagaccaactacgtggtcatcatctgcgcctgccgcagcgcggccatcgactcctgcatcgccgtcaccggaaaataccaagcccggattctttccgagagccttctcactcctgcggagtaccagaaggaagtcaattacgagctggttacggggaaggtagactcgctgggggccttctttagcaccctctgtccagagggtgacattgacattttgctggacaaatttcaccaggaaaatcaaggccatatttcttcctcactcgctgcctcttctgtcactaaagcagcatccctggatgtcagtgggacaccggtgtgcacaagttacaatctggagccacacagcatccggcccttccagctggcagtagcgcagaagctcctctcccatgtgtgttccattgcggattccagcacccaaaatctggacctgggatcctttgagaaggtggactttctcatttgcattcccccctcagaagtgacctaccagcagactctgctccatgtgtggcattcaggggttttgctggagcttggtctgaagaaagagcacatgacgaagcagagggtggaacagtatgttctgaagctagacacggaggcacagacaaaatttaaggcttttctgcaaaactccttccagaacccgcatacactttttgtcctaatccatgaccatgcgcactgggatcttgtgagtagcactgttcataacctctattctcaaagtgacccgtcggtgggattggtggaccgattgctcaactgcagggaggtgaaggaggcccccaacattgtgacacttcacgtgacctccttcccgtatgcactgcagacacagcacaccctcatcagcccctacaacgagatccactggcctgcctcctgcagtaatggagtggacttatatcatgaaaataagaagtacttcgggctgtcggagtttattgaatccaccctttcaggacacagcctccccttgctcagatacgatagctcctttgaggccatggtcactgcattaggaaaaaggttcccccgcctgcacagcgcggtgatcaggacctttgttctcgtgcagcactacgcggccgccctgatggccgtaagcggcctcccgcagatgaagaactacacgtcggtggagacgctggagatcacgcagaacctcctcaactccccgaagcagtgcccctgcggccacgggctcatggtcctgctgcgggtgccctgttcgcccctggcggtggtggcctatgagcggctggcccacgtgcgggcccggctggcgctggaggagcactttgagatcatcctgggcagtcccagctcaggcgtcaccgtggggaagcacttcgtaaagcagctcaggatgtggcagaaaattgaggatgtggagtggagaccccagacttacttggagctggagggtctgccttgcatcctgatcttcagtgggatggacccgcatggggagtccttgccgaggtctttgaggtactgtgacctgcgattgataaactcctcctgcttggtgagaacagccttggagcaggagctgggcctggctgcctactttgtgagcaacgaggttcccttggagaagggggctaggaacgaggccttggagagtgatgctgagaagctgagcagcacagacaacgaggatgaggagctggggacagaaggctctacctcggagaagagaagccccatgaaaagggagaggtcccgctcccacgactcagcatcctcatccctctcctccaaggcttccggttcagcgctcggtggcgagtcctcggctcagcccacagcactcccccagggagagcatgccaggtcgccccagccccgtggccccgcagaggagggcagagcccctggtgagaaacagaggccccgggcaagtcaggggccaccctcggccatcagcaggcacagtcccgggccgacgccccagcccgactgtagcctcaggaccggccagaggagcgtccaggtgtcggtcacctcgtcgtgctcccagctgtcctcctcctcgggctcatcctcctcatccgtggcgcccgctgccggcacgtgggtcctgcaggcctcccagtgctccttgaccaaggcctgccgccagccacccattgtcttcttgcccaagctcgtgtacgacatggttgtgtccactgacagcagtggcctgcccaaggccgcctccctcctgccctccccctcggtcatgtgggccagctctttccgccccctgctcagcaagaccatgacatccaccgagcagtccctctactaccggcagtggacggtgccccggcccagccacatggactacggcaaccgggccgagggccgcgtggacggcttccacccccgcaggctgctgctcagcggcccccctcagatcgggaagacaggtgcctacctgcagttcctcagtgtcctgtccaggatgcttgttcggctcacagaagtggatgtctatgacgaggaggagatcaatatcaacctcagagaagaatctgactggcattatctccagcttagcgacccctggccagacctggagctgttcaagaagttgccctttgactacatcattcacgacccgaagtatgaagatgccagcctgatttgttcgcactatcagggtataaagagtgaagacagagggatgtcccggaagccggaggacctttatgtgcggcgtcagacggcacggatgagactgtccaagtacgcagcgtacaacacttaccaccactgtgagcagtgccaccagtacatgggcttccacccccgctaccagctgtatgagtccaccctgcacgcctttgccttctcttactccatgctaggagaggagatccagctgcacttcatcatccccaagtccaaggagcaccactttgtcttcagccaacctggaggccagctggagagcatgcgactacccctcgtgacagacaagagccatgaatatataaaaagtccgacattcactccaaccaccggccgtcacgaacatgggctctttaatctgtaccacgcaatggacggtgccagccatttgcacgtgctggttgtcaaggaatacgagatggcaatttataagaaatattggcccaaccacatcatgctggtgctccccagtatcttcaacagtgctggagttggtgctgctcatttcctcatcaaggagctgtcctaccataacctggagctcgagcggaaccggcaggaggagctgggaatcaagccgcaggacatctggcctttcattgtgatctctgatgactcctgcgtgatgtggaacgtggtggatgtcaactctgctggggagagaagcagggagttctcctggtcggaaaggaacgtgtctttgaagcacatcatgcagcacatcgaggcggcccccgacatcatgcactacgccctgctgggcctgcggaagtggtccagcaagacccgggccagcgaggtgcaagagcccttctcccgctgccacgtgcacaacttcatcatcctgaacgtggacctgacccagaacgtgcagtacaaccagaaccggttcctgtgtgacgatgtagacttcaacctgcgggtgcacagcgccggcctcctgctctgccggttcaaccgcttcagcgtgatgaagaagcagatcgtggtgggcggccacaggtccttccacatcacatccaaggtgtctgataactctgccgcggtcgtgccggcccagtacatctgtgccccggacagcaagcacacgttcctcgcagcgcccgcccagctcctgctggagaagttcctgcagcaccacagccacctcttcttcccgctgtccctgaagaaccatgaccacccagtgctgtctgtcgactgttacctgaacctgggatctcagatttctgtttgctatgtgagctccaggccccactctttaaacatcagctgctcggacttgctgttcagtgggctgctgctgtacctctgtgactcttttgtgggagctagctttttgaaaaagtttcattttctgaaaggtgcgacgttgtgtgtcatctgtcaggaccggagctcactgcgccagacggtcgtccgcctggagctcgaggacgagtggcagttccggctgcgcgatgagttccagaccgccaatgccagggaagaccggccgctcttttttctgacgggacgacacatctga

SEQ ID NO: 4 - DNA, 21 bp, Artificial Sequence
  Sense strand of GREB1 #1 siRNA
gccauucgugugcuuccautt

SEQ ID NO: 5 - DNA, 21 bp, Artificial Sequence
  Antisense strand of GREB1 #1 siRNA
auggaagcacacgaauggctt

SEQ ID NO: 6 - DNA, 21 bp, Artificial Sequence
  Sense strand of GREB1 #2 siRNA
ccuccuacaaagcaauauutt

SEQ ID NO: 7 - DNA, 21 bp, Artificial Sequence
  Antisense strand of  GREB1 #2
aauauugcuuuguaggaggtt

SEQ ID NO: 8 - DNA, 59 bp, Artificial Sequence
  Base sequence coding mouse Greb1 shRNA
ccggctactttgttagcaatgatatctcgagatatcattgctaacaaagtagttttttg

SEQ ID NO: 9 - DNA, 21 bp, Artificial Sequence
  Sense strand of control siRNA
cagucgcguuugcgacuggtt

SEQ ID NO: 10 - DNA, 21 bp, Artificial Sequence
  Antisense strand of control siRNA
ccagucgcaaacgcgacugtt

SEQ ID NO: 11 - DNA, 21 bp, Artificial Sequence
  Sense strand of beta-catenin #1 siRNA
cccacuaauguccagcguutt

SEQ ID NO: 12 - DNA, 21 bp, Artificial Sequence
  Antisense strand of beta-catenin #1 siRNA
aacgcuggacauuagugggtt

SEQ ID NO: 13 - DNA, 21 bp, Artificial Sequence
  Sense strand of beta-catenin #2 siRNA
gcauaaccuuucccaucautt

SEQ ID NO: 14 - DNA, 21 bp, Artificial Sequence
  Antisense strand of beta-catenin #2 siRNA
augaugggaaagguuaugctt

SEQ ID NO: 15 - DNA, 21 bp, Artificial Sequence
  Sense strand of c-Met siRNA
gcaucagaaccagaggcuutt

SEQ ID NO: 16 - DNA, 21 bp, Artificial Sequence
  Antisense strand of c-Met siRNA
aagccucugguucugaugctt

SEQ ID NO: 17 - DNA, 21 bp, Artificial Sequence
  Sense strand of p15 siRNA
ggaauaaccuuccauacautt

SEQ ID NO: 18 - DNA, 21 bp, Artificial Sequence
  Antisense strand of p15 siRNA
auguauggaagguuauucctt

SEQ ID NO: 19 - DNA, 21 bp, Artificial Sequence
  Sense strand of YAP #1 siRNA
gacaucuucuggucagagatt

SEQ ID NO: 20 - DNA, 21 bp, Artificial Sequence
  Antisense strand of YAP #1 siRNA
ucucugaccagaagauguctt

SEQ ID NO: 21 - DNA, 21 bp, Artificial Sequence
  Sense strand of YAP #2 siRNA
cuggucagagauacuucuutt

SEQ ID NO: 22 - DNA, 21 bp, Artificial Sequence
  Antisense strand of YAP #2 siRNA
aagaaguaucucugaccagtt

SEQ ID NO: 23 - DNA, 21 bp, Artificial Sequence
  Sense strand of TAZ #1 siRNA
acguugacuuaggaacuuutt

SEQ ID NO: 24 - DNA, 21 bp, Artificial Sequence
  Antisense strand of TAZ #1 siRNA
aaaguuccuaagucaacgutt

SEQ ID NO: 25 - DNA, 21 bp, Artificial Sequence
  Sense strand of TAZ #2 siRNA
agguacuuccucaaucacatt

SEQ ID NO: 26 - DNA, 21 bp, Artificial Sequence
  Antisense strand of AZ #2 siRNA
ugugauugaggaaguaccutt

SEQ ID NO: 27 - DNA, 20 bp, Artificial Sequence
  Forward primer for human GREB1
gtgctctaccggctcaagtt

SEQ ID NO: 28 - DNA, 20 bp, Artificial Sequence
  Reverse primer for human GREB1
accgagtccaccacgtagat

SEQ ID NO: 29 - DNA, 22 bp, Artificial Sequence
  Forward primer for human Axin2
ctggctccagaagatcacaaag

SEQ ID NO: 30 - DNA, 22 bp, Artificial Sequence
  Reverse primer for human Axin2
catcctcccagatctcctcaaa

SEQ ID NO: 31 - DNA, 20 bp, Artificial Sequence
  Forward primer for human DLK1
tggcttctcaggcaatttct

SEQ ID NO: 32 - DNA, 20 bp, Artificial Sequence
  Reverse primer for human DLK1
ggcttgcacagacactcgta

SEQ ID NO: 33 - DNA, 20 bp, Artificial Sequence
  Forward primer for human AFP
agcttggtggtggatgaaac

SEQ ID NO: 34 - DNA, 20 bp, Artificial Sequence
  Reverse primer for human AFP
ccctcttcagcaaagcagac

SEQ ID NO: 35 - DNA, 20 bp, Artificial Sequence
  Forward primer for human PEG3
tcacttccagcacagcattc

SEQ ID NO: 36 - DNA, 20 bp, Artificial Sequence
  Reverse primer for human PEG3
cctcagccagtgtgggtatt

SEQ ID NO: 37 - DNA, 20 bp, Artificial Sequence
  Forward primer for human PRLR
tcatgatggtcaatgccact

SEQ ID NO: 38 - DNA, 20 bp, Artificial Sequence
  Reverse primer for human PRLR
gcgtgaaccaaccagttttt

SEQ ID NO: 39 - DNA, 20 bp, Artificial Sequence
  Forward primer for human XBP1
tcacccctccagaacatctc

SEQ ID NO: 40 - DNA, 20 bp, Artificial Sequence
  Reverse primer for human XBP1
acagagaaagggaggctggt

SEQ ID NO: 41 - DNA, 20 bp, Artificial Sequence
  Forward primer for human PAI-1
cacaccagctccactgaaga

SEQ ID NO: 42 - DNA, 20 bp, Artificial Sequence
  Reverse primer for human PAI-1
ctccatcacaggagcagaca

SEQ ID NO: 43 - DNA, 20 bp, Artificial Sequence
  Forward primer for human SNAIL2
ctttttcttgccctcactgc

SEQ ID NO: 44 - DNA, 20 bp, Artificial Sequence
  Reverse primer for human SNAIL2
acagcagccagattcctcat

SEQ ID NO: 45 - DNA, 20 bp, Artificial Sequence
  Forward primer for human TGFb1
cacgtggagctgtaccagaa

SEQ ID NO: 46 - DNA, 20 bp, Artificial Sequence
  Reverse primer for human TGFb1
tgcagtgtgttatccctgct

SEQ ID NO: 47 - DNA, 20 bp, Artificial Sequence
  Forward primer for human p15
gaccgggaataaccttccat

SEQ ID NO: 48 - DNA, 20 bp, Artificial Sequence
  Reverse primer for human p15
caccaggtccagtcaaggat

SEQ ID NO: 49 - DNA, 20 bp, Artificial Sequence
  Forward primer for human p21
atgaaattcaccccctttcc

SEQ ID NO: 50 - DNA, 20 bp, Artificial Sequence
  Reverse primer for human p21
ccctaggctgtgctcacttc

SEQ ID NO: 51 - DNA, 20 bp, Artificial Sequence
  Forward primer for human p27
acccctagagggcaagtacg

SEQ ID NO: 52 - DNA, 20 bp, Artificial Sequence
  Reverse primer for human p27
atcagtctttgggtccacca

SEQ ID NO: 53 - DNA, 20 bp, Artificial Sequence
  Forward primer for human ANKRD1
acgccaaagacagagaagga

SEQ ID NO: 54 - DNA, 20 bp, Artificial Sequence
  Reverse primer for human ANKRD1
ttctgccagtgtagcaccag

SEQ ID NO: 55 - DNA, 20 bp, Artificial Sequence
  Forward primer for human Cyr61
ctccctgtttttggaatgga

SEQ ID NO: 56 - DNA, 20 bp, Artificial Sequence
  Reverse primer for human Cyr61
tggtcttgctgcatttcttg

SEQ ID NO: 57 - DNA, 30 bp, Artificial Sequence
  Forward primer for human specific beta-catenin
tagaaacagctcgttgtaccgctgggacct

SEQ ID NO: 58 - DNA, 32 bp, Artificial Sequence
  Reverse primer for human specific beta-catenin
gcactgccattttagctccttcttgatgtaat

SEQ ID NO: 59 - DNA, 27 bp, Artificial Sequence
  Forward primer for human specific YAP
ccaccagtccaccagtgcagcagaata

SEQ ID NO: 60 - DNA, 30 bp, Artificial Sequence
  Reverse primer for human specific YAP
gcagtcgcatctgttgctgctggttggagt

SEQ ID NO: 61 - DNA, 30 bp, Artificial Sequence
  Forward primer for human specific Met
tggactcaacagatctgtctgcctgcaatc

SEQ ID NO: 62 - DNA, 30 bp, Artificial Sequence
  Reverse primer for human specific Met
aatgtactgtattgtgttgtcccgtggcca

SEQ ID NO: 63 - DNA, 20 bp, Artificial Sequence
  Forward primer for human GAPDH
tcctgcaccaccaactgctt

SEQ ID NO: 64 - DNA, 19 bp, Artificial Sequence
  Reverse primer for human GAPDH
tggcagtgatggcatggac

SEQ ID NO: 65 - DNA, 20 bp, Artificial Sequence
  Forward primer for human UBC
cctggtgctccgtcttagag

SEQ ID NO: 66 - DNA, 20 bp, Artificial Sequence
  Reverse primer for human UBC
tttcccagcaaagatcaacc

SEQ ID NO: 67 - DNA, 30 bp, Artificial Sequence
  Forward primer for human Met
agagctggtccaggcagtgcagcatgtagt

SEQ ID NO: 68 - DNA, 30 bp, Artificial Sequence
  Reverse primer for human Met
aatctttcatgatgattccctcggtcagaa

SEQ ID NO: 69 - DNA, 20 bp, Artificial Sequence
  Forward primer for mouse GREB1
tgattcggctgacagaagtg

SEQ ID NO: 70 - DNA, 20 bp, Artificial Sequence
  Reverse primer for mouse GREB1
tgatggtctgagggtgtgaa

SEQ ID NO: 71 - DNA, 30 bp, Artificial Sequence
  Forward primer for mouse AFP
gcactgtccaagcaaagctgcgctctctac

SEQ ID NO: 72 - DNA, 30 bp, Artificial Sequence
  Reverse primer for mouse AFP
gctgataccagagttcacagggcttgcttc

SEQ ID NO: 73 - DNA, 30 bp, Artificial Sequence
  Forward primer for mouse GPC3
gaaccatgtctgtgcccaagggtaaagttc

SEQ ID NO: 74 - DNA, 30 bp, Artificial Sequence
  Reverse primer for mouse GPC3
cgctgtgagaggtggtgatctcgttgtcct

SEQ ID NO: 75 - DNA, 30 bp, Artificial Sequence
  Forward primer for mouse TACSTD1
gatctggaccccgggcagactctgatttac

SEQ ID NO: 76 - DNA, 30 bp, Artificial Sequence
  Reverse primer for mouse TACSTD1
cagcactcagcacggctaggcattaagctc

SEQ ID NO: 77 - DNA, 26 bp, Artificial Sequence
  Forward primer for mouse DLK1
tggctgtgtcaatggagtctgcaagg

SEQ ID NO: 78 - DNA, 27 bp, Artificial Sequence
  Reverse primer for mouse DLK1
tgctggcagggagaaccattgatcacg

SEQ ID NO: 79 - DNA, 30 bp, Artificial Sequence
  Forward primer for mouse PEG3
actccctaccttttggtgagttgcttgcag

SEQ ID NO: 80 - DNA, 30 bp, Artificial Sequence
  Reverse primer for mouse PEG3
cttggatgaaacgttcttgggcataactgg

SEQ ID NO: 81 - DNA, 30 bp, Artificial Sequence
  Forward primer for mouse BEX1
aggcaaggataggcccaggagtaatggagt

SEQ ID NO: 82 - DNA, 30 bp, Artificial Sequence
  Reverse primer for mouse BEX1
ctccccaaccctctgcatcaggtcccatct

SEQ ID NO: 83 - DNA, 30 bp, Artificial Sequence
  Forward primer for mouse MEG3
tatctggacattgaagcttggaaagccagt

SEQ ID NO: 84 - DNA, 30 bp, Artificial Sequence
  Reverse primer for mouse MEG3
ttcatgaccacagcccatggtatcacacag

SEQ ID NO: 85 - protein, 9 aa, Artificial Sequence
  HA
YPYDVPDYA

SEQ ID NO: 86 - protein, 8 aa, Artificial Sequence
  FLAG
DYKDDDDK

SEQ ID NO: 87 - protein, 265 aa, Artificial Sequence
  GFP
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGLRSRAQASNSAVDGTAGPGSTGSR

SEQ ID NO: 88 - protein, 1944 aa, Artificial Sequence
  Mouse GREB1 delta 310-319 (delta NLS)
MGNSYAGQLKSTRFEEVLHNSIEASLRSNTLVPRPIFSQLYLEAEQQLSSLEGGSRADNEEEEEDGEGGLEPSSPPNAYQLPPPPEGCCTTDGFCQAGKDLRLVSISSEPIEVPAGFLLVGAKSPSLPDHLLVCAVDKRFLPDDNGHNALLGFSGNCVGCGKKGFCYFTEFSNHINLKLTTQPKKQKHLKYYLVRNAQGALTKGPLICWKGSEFRGRQNSTNTCSSSLFPPLESSGSLAAFPTEPVPGTNPSVPVGAQQAGPASDHPSVTTATGPAVFNGKDSPKHPQLVKSSLSALPRPSALGILPNSSPESKSTTDGGFIQGGGNRAKHEGTSIPCVPQAGLVGPASVTFPVVASGEPVSVPDNLLKICKAKPVIFKGHGNFPYLCGNLNDVVVSPLLYTCYQNSQSLARAYEQHGASTMQPISEETQLLLTVYYLVQLAADQVPLMEDLEQIFLRSWRESHLTEIRQYQQAPPQPFPPATSTAAPVTSAQLPWLAGLAASSCNDSVHVIECAYSLAEGLSEMFRLLIEGKLSKTNYVVIICACRNAAIDSCIAVTGKYQARILSESLLSPAEYQREVHYELVTGKVDSLGTFFSSLCPEGDIDILLDKFHQENQGHVSSSFTASSTKKTAVLDASGVPVCTSYHQEPRGVRPFQLAVAQKLLSHVCSIADSSTQNLDLGSFEKVDFLICIPPSEVTYQQTVFHVWHSGVLLELGLEKEPVTKQRAEQHVLKLDTEAQARFKAFLQNSFQNPHTLFVLIHDHAHWDLVSSAVHNIYSQSDPSVGLVDRLLNCREVKEAPNIVTLHVTSFPYALQTQHTLISPYNEIHWPISFSNGVDLYHESKKYFGLSEFIDSTLSGHSLPLLRYDSSFEAMVTALGKRFPRLHSAVIRTFVLVQHYAAAMMAVSGLPQMKNHTSVETLEITQNLLNSPKQCPCGHGLMVLLRVPCSPLAAVAYERLAHVRARLALEEHFEIILGHPSSGITVGKHFVKQLKMWQKIEDAEWRPQTYLELEGLPCILIFSGMDPHGESLPRSLRYCDLRLINSSCLVRTALEQELGLAAYFVSNDIPLEKGPKNEALESDGEKLSSTDEDEEAGTEGCFEAGSTSEQRGPVKRERSHSHDSASSSLSSRASGSVLYGESLAQPSGPPQGELTRSPPSCGPAEEGRAPGEIQRLRVSQGSTVISRHSPGLVPQPDSSLRTGRRSLQVPAAPSSQLSSSSGSSSTCAVPTANVLVLQASQCSMAKACRQPPIVFLPKLVYDMLLSTDSSGLPKSASLLPSPSVMWTSSFRPLLSKMMTSTEQSLYYRQWTVPRPSHMDYGNRAEGRVDSFHPRRLLLSGPPQIGKTGAYLQFLSILSRMLIRLTEVDVYDEEEINTSFREESEWRYLQLADPWPDLELFQKMPFDYIIHDPKYEDASLICSHPQTIKSEDRGMSRKPEDLYVRRQTARMRLSKYAAYNTYHHCEQCQQYMGFHPHYQLSESTLHVFAFSCSMLGEEVQLHFIIPKSKEHHFVFSQPGGQLESMRLPLVTDKSHEHIKSPTFTPTTGRHEHGLFNLYHAMDGANHLHVLVVKEYEMAIYKKYWPNHIMLVLPSIFNSAGVGAAHFLIKELCYHNLELERNRQEELGVKPQDVWPFIVIADDSCVMWNVADVDCAGERSREFSWSERNVSLKYIMLHIEASPNITHYALLGMRKWASKTRGREVQEPFSRCHVHDFIILNVDLTQNVQYNQNRFTCDDVDFNLRVHSAGLLLCRFNRFSVMKKQIAVGGHRSFHITSKVSDSSVAIVPSQYICAPDSKHTFLAAPAQLLLEKFLQYHSHRFFPLSLKNHSHPVLSVDCYLNLGPQISVCYVSSRPHSLNISCSDMVFSGLLLYLCDSFVGASFLKKFHFLKGATLCVICQDRNSLRQTVVRLELEDEWQFRLRDEFQTANAKEDRPLFFLTARHI

SEQ ID NO: 89 - protein, 666 aa, Artificial Sequence
  Mouse GREB1 1-666(N)
MGNSYAGQLKSTRFEEVLHNSIEASLRSNTLVPRPIFSQLYLEAEQQLSSLEGGSRADNEEEEEDGEGGLEPSSPPNAYQLPPPPEGCCTTDGFCQAGKDLRLVSISSEPIEVPAGFLLVGAKSPSLPDHLLVCAVDKRFLPDDNGHNALLGFSGNCVGCGKKGFCYFTEFSNHINLKLTTQPKKQKHLKYYLVRNAQGALTKGPLICWKGSEFRGRQNSTNTCSSSLFPPLESSGSLAAFPTEPVPGTNPSVPVGAQQAGPASDHPSVTTATGPAVFNGKDSPKHPQLVKSSLSALPRPSALGILPNSGPPKKRHKGWSPESKSTTDGGFIQGGGNRAKHEGTSIPCVPQAGLVGPASVTFPVVASGEPVSVPDNLLKICKAKPVIFKGHGNFPYLCGNLNDVVVSPLLYTCYQNSQSLARAYEQHGASTMQPISEETQLLLTVYYLVQLAADQVPLMEDLEQIFLRSWRESHLTEIRQYQQAPPQPFPPATSTAAPVTSAQLPWLAGLAASSCNDSVHVIECAYSLAEGLSEMFRLLIEGKLSKTNYVVIICACRNAAIDSCIAVTGKYQARILSESLLSPAEYQREVHYELVTGKVDSLGTFFSSLCPEGDIDILLDKFHQENQGHVSSSFTASSTKKTAVLDASGVPVCTSYHQEPRGVRPF

SEQ ID NO: 90 - protein, 668 aa, Artificial Sequence
  Mouse GREB1 667-1333(I)
MQLAVAQKLLSHVCSIADSSTQNLDLGSFEKVDFLICIPPSEVTYQQTVFHVWHSGVLLELGLEKEPVTKQRAEQHVLKLDTEAQARFKAFLQNSFQNPHTLFVLIHDHAHWDLVSSAVHNIYSQSDPSVGLVDRLLNCREVKEAPNIVTLHVTSFPYALQTQHTLISPYNEIHWPISFSNGVDLYHESKKYFGLSEFIDSTLSGHSLPLLRYDSSFEAMVTALGKRFPRLHSAVIRTFVLVQHYAAAMMAVSGLPQMKNHTSVETLEITQNLLNSPKQCPCGHGLMVLLRVPCSPLAAVAYERLAHVRARLALEEHFEIILGHPSSGITVGKHFVKQLKMWQKIEDAEWRPQTYLELEGLPCILIFSGMDPHGESLPRSLRYCDLRLINSSCLVRTALEQELGLAAYFVSNDIPLEKGPKNEALESDGEKLSSTDEDEEAGTEGCFEAGSTSEQRGPVKRERSHSHDSASSSLSSRASGSVLYGESLAQPSGPPQGELTRSPPSCGPAEEGRAPGEIQRLRVSQGSTVISRHSPGLVPQPDSSLRTGRRSLQVPAAPSSQLSSSSGSSSTCAVPTANVLVLQASQCSMAKACRQPPIVFLPKLVYDMLLSTDSSGLPKSASLLPSPSVMWTSSFRPLLSKMMTSTEQSLYYRQWTVPRPSHMDYGNR

SEQ ID NO: 91 - protein, 622 aa, Artificial Sequence
  Mouse GREB1 1334-1954(C)
MAEGRVDSFHPRRLLLSGPPQIGKTGAYLQFLSILSRMLIRLTEVDVYDEEEINTSFREESEWRYLQLADPWPDLELFQKMPFDYIIHDPKYEDASLICSHPQTIKSEDRGMSRKPEDLYVRRQTARMRLSKYAAYNTYHHCEQCQQYMGFHPHYQLSESTLHVFAFSCSMLGEEVQLHFIIPKSKEHHFVFSQPGGQLESMRLPLVTDKSHEHIKSPTFTPTTGRHEHGLFNLYHAMDGANHLHVLVVKEYEMAIYKKYWPNHIMLVLPSIFNSAGVGAAHFLIKELCYHNLELERNRQEELGVKPQDVWPFIVIADDSCVMWNVADVDCAGERSREFSWSERNVSLKYIMLHIEASPNITHYALLGMRKWASKTRGREVQEPFSRCHVHDFIILNVDLTQNVQYNQNRFTCDDVDFNLRVHSAGLLLCRFNRFSVMKKQIAVGGHRSFHITSKVSDSSVAIVPSQYICAPDSKHTFLAAPAQLLLEKFLQYHSHRFFPLSLKNHSHPVLSVDCYLNLGPQISVCYVSSRPHSLNISCSDMVFSGLLLYLCDSFVGASFLKKFHFLKGATLCVICQDRNSLRQTVVRLELEDEWQFRLRDEFQTANAKEDRPLFFLTARHI

SEQ ID NO: 92 - protein, 1287 aa, Artificial Sequence
  Mouse GREB1 delta 667-1333(delta I)
MGNSYAGQLKSTRFEEVLHNSIEASLRSNTLVPRPIFSQLYLEAEQQLSSLEGGSRADNEEEEEDGEGGLEPSSPPNAYQLPPPPEGCCTTDGFCQAGKDLRLVSISSEPIEVPAGFLLVGAKSPSLPDHLLVCAVDKRFLPDDNGHNALLGFSGNCVGCGKKGFCYFTEFSNHINLKLTTQPKKQKHLKYYLVRNAQGALTKGPLICWKGSEFRGRQNSTNTCSSSLFPPLESSGSLAAFPTEPVPGTNPSVPVGAQQAGPASDHPSVTTATGPAVFNGKDSPKHPQLVKSSLSALPRPSALGILPNSGPPKKRHKGWSPESKSTTDGGFIQGGGNRAKHEGTSIPCVPQAGLVGPASVTFPVVASGEPVSVPDNLLKICKAKPVIFKGHGNFPYLCGNLNDVVVSPLLYTCYQNSQSLARAYEQHGASTMQPISEETQLLLTVYYLVQLAADQVPLMEDLEQIFLRSWRESHLTEIRQYQQAPPQPFPPATSTAAPVTSAQLPWLAGLAASSCNDSVHVIECAYSLAEGLSEMFRLLIEGKLSKTNYVVIICACRNAAIDSCIAVTGKYQARILSESLLSPAEYQREVHYELVTGKVDSLGTFFSSLCPEGDIDILLDKFHQENQGHVSSSFTASSTKKTAVLDASGVPVCTSYHQEPRGVRPFAEGRVDSFHPRRLLLSGPPQIGKTGAYLQFLSILSRMLIRLTEVDVYDEEEINTSFREESEWRYLQLADPWPDLELFQKMPFDYIIHDPKYEDASLICSHPQTIKSEDRGMSRKPEDLYVRRQTARMRLSKYAAYNTYHHCEQCQQYMGFHPHYQLSESTLHVFAFSCSMLGEEVQLHFIIPKSKEHHFVFSQPGGQLESMRLPLVTDKSHEHIKSPTFTPTTGRHEHGLFNLYHAMDGANHLHVLVVKEYEMAIYKKYWPNHIMLVLPSIFNSAGVGAAHFLIKELCYHNLELERNRQEELGVKPQDVWPFIVIADDSCVMWNVADVDCAGERSREFSWSERNVSLKYIMLHIEASPNITHYALLGMRKWASKTRGREVQEPFSRCHVHDFIILNVDLTQNVQYNQNRFTCDDVDFNLRVHSAGLLLCRFNRFSVMKKQIAVGGHRSFHITSKVSDSSVAIVPSQYICAPDSKHTFLAAPAQLLLEKFLQYHSHRFFPLSLKNHSHPVLSVDCYLNLGPQISVCYVSSRPHSLNISCSDMVFSGLLLYLCDSFVGASFLKKFHFLKGATLCVICQDRNSLRQTVVRLELEDEWQFRLRDEFQTANAKEDRPLFFLTARHI

SEQ ID NO: 93 - protein, 467 aa, Homo sapiens
MSSILPFTPPVVKRLLGWKKSAGGSGGAGGGEQNGQEEKWCEKAVKSLVKKLKKTGRLDELEKAITTQNCNTKCVTIPSTCSEIWGLSTPNTIDQWDTTGLYSFSEQTRSLDGRLQVSHRKGLPHVIYCRLWRWPDLHSHHELKAIENCEYAFNLKKDEVCVNPYHYQRVETPVLPPVLVPRHTEILTELPPLDDYTHSIPENTNFPAGIEPQSNYIPETPPPGYISEDGETSDQQLNQSMDTGSPAELSPTTLSPVNHSLDLQPVTYSEPAFWCSIAYYELNQRVGETFHASQPSLTVDGFTDPSNSERFCLGLLSNVNRNATVEMTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSVRCSSMS

SEQ ID NO: 94 - protein, 265 aa, Artificial Sequence
  Human Smad2 1-265 (N)
MSSILPFTPPVVKRLLGWKKSAGGSGGAGGGEQNGQEEKWCEKAVKSLVKKLKKTGRLDELEKAITTQNCNTKCVTIPSTCSEIWGLSTPNTIDQWDTTGLYSFSEQTRSLDGRLQVSHRKGLPHVIYCRLWRWPDLHSHHELKAIENCEYAFNLKKDEVCVNPYHYQRVETPVLPPVLVPRHTEILTELPPLDDYTHSIPENTNFPAGIEPQSNYIPETPPPGYISEDGETSDQQLNQSMDTGSPAELSPTTLSPVNHSLDLQP

SEQ ID NO: 95 - protein, 202 aa, Artificial Sequence
  Human Smad2 266-467
VTYSEPAFWCSIAYYELNQRVGETFHASQPSLTVDGFTDPSNSERFCLGLLSNVNRNATVEMTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSVRCSSMS

SEQ ID NO: 96 - protein, 425 aa, Homo sapiens
MSSILPFTPPIVKRLLGWKKGEQNGQEEKWCEKAVKSLVKKLKKTGQLDELEKAITTQNVNTKCITIPRSLDGRLQVSHRKGLPHVIYCRLWRWPDLHSHHELRAMELCEFAFNMKKDEVCVNPYHYQRVETPVLPPVLVPRHTEIPAEFPPLDDYSHSIPENTNFPAGIEPQSNIPETPPPGYLSEDGETSDHQMNHSMDAGSPNLSPNPMSPAHNNLDLQPVTYCEPAFWCSISYYELNQRVGETFHASQPSMTVDGFTDPSNSERFCLGLLSNVNRNAAVELTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQMGSPSIRCSSVS

SEQ ID NO: 97 - protein, 552 aa, Homo sapiens
MDNMSITNTPTSNDACLSIVHSLMCHRQGGESETFAKRAIESLVKKLKEKKDELDSLITAITTNGAHPSKCVTIQRTLDGRLQVAGRKGFPHVIYARLWRWPDLHKNELKHVKYCQYAFDLKCDSVCVNPYHYERVVSPGIDLSGLTLQSNAPSSMMVKDEYVHDFEGQPSLSTEGHSIQTIQHPPSNRASTETYSTPALLAPSESNATSTANFPNIPVASTSQPASILGGSHSEGLLQIASGPQPGQQQNGFTGQPATYHHNSTTTWTGSRTAPYTPNLPHHQNGHLQHHPPMPPHPGHYWPVHNELAFQPPISNHPAPEYWCSIAYFEMDVQVGETFKVPSSCPIVTVDGYVDPSGGDRFCLGQLSNVHRTEAIERARLHIGKGVQLECKGEGDVWVRCLSDHAVFVQSYYLDREAGRAPGDAVHKIYPSAYIKVFDLRQCHRQMQQQAATAQAAAAAQAAAVAGNIPGPGSVGGIAPAISLSAAAGIGVDDLRRLCILRMSFVKGWGPDYPRQSIKETPCWIEIHLHRALQLLDEVLHTMPIADPQPLD

SEQ ID NO: 98 - protein, 426 aa, Homo sapiens
MFRTKRSALVRRLWRSRAPGGEDEEEGAGGGGGGGELRGEGATDSRAHGAGGGGPGRAGCCLGKAVRGAKGHHHPHPPAAGAGAAGGAEADLKALTHSVLKKLKERQLELLLQAVESRGGTRTACLLLPGRLDCRLGPGAPAGAQPAQPPSSYSLPLLLCKVFRWPDLRHSSEVKRLCCCESYGKINPELVCCNPHHLSRLCELESPPPPYSRYPMDFLKPTADCPDAVPSSAETGGTNYLAPGGLSDSQLLLEPGDRSHWCVVAYWEEKTRVGRLYCVQEPSLDIFYDLPQGNGFCLGQLNSDNKSQLVQKVRSKIGCGIQLTREVDGVWVYNRSSYPIFIKSATLDNPDSRTLLVHKVFPGFSIKAFDYEKAYSLQRPNDHEFMQQPWTGFTVQISFVKGWGQCYTRQFISSCPCWLEVIFNSR

SEQ ID NO: 99 - protein, 218 aa, Homo sapiens
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPK

SEQ ID NO: 100 - protein, 165 aa, Homo sapiens
TPPVVKRLLGWKKSAGGSGGAGGGEQNGQEEKWCEKAVKSLVKKLKKTGRLDELEKAITTQNCNTKCVTIPSTCSEIWGLSTPNTIDQWDTTGLYSFSEQTRSLDGRLQVSHRKGLPHVIYCRLWRWPDLHSHHELKAIENCEYAFNLKKDEVCVNPYHYQRVET

SEQ ID NO: 101 - protein, 191 aa, Homo sapiens
VTYSEPAFWCSIAYYELNQRVGETFHASQPSLTVDGFTDPSNSERFCLGLLSNVNRNATVEMTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLDKVLTQM

SEQ ID NO: 102 - DNA, 20 bp, Artificial Sequence
  Target sequence on human GREB1
cttctcggtgttgaagccga

SEQ ID NO: 103 - DNA, 20 bp, Artificial Sequence
  Target sequence of gRNA for human Smad2
tatattgccgattatggcgc

SEQ ID NO: 104 - DNA, 20 bp, Artificial Sequence
  Target sequence of gRNA for human Smad3
ggaatgtctccccgacgcgc

SEQ ID NO: 105 - DNA, 26 bp, Artificial Sequence
  Forward primer for Tcf4 binding site of human GREB1
tggatgatacacagattgctaccaac

SEQ ID NO: 106 - DNA, 25 bp, Artificial Sequence
  Reverse primer for Tcf4 binding site of human GREB1
gcttgcctttctccctgcactaagc

SEQ ID NO: 107 - DNA, 28 bp, Artificial Sequence
  Forward primer for human PAI-1 exon 2
accgcaacgtggttttctcaccctatgg

SEQ ID NO: 108 - DNA, 28 bp, Artificial Sequence
  Reverse primer for human PAI-1 exon 2
aatcttgaatcccatagctgcttgaatc

SEQ ID NO: 109 - protein, 24 aa, Artificial Sequence
  3 copies of NLS sequence
PKKKRKVDPKKKRKVDPKKKRKVD